SEQUENCE LISTING (62 sequences)

SEQ ID NO: 1 - protein, 1368 aa, Streptococcus pyogenes
  MISC_FEATURE (1)..(1368): AKP81606.1
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNSLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 1300 aa, Francisella tularensis
  MISC_FEATURE (1)..(1300): subsp. novicida U112 AJI61006.1
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 3 - protein, 15 aa, Artificial Sequence
  Synthetic
IGLDIGTNSVGWAVI

SEQ ID NO: 4 - protein, 8 aa, Artificial Sequence
  Synthetic
IVIEMARE

SEQ ID NO: 5 - protein, 27 aa, Artificial Sequence
  Synthetic
DVDHIVPQSFLKDDSIDNKVLTRSDKN

SEQ ID NO: 6 - protein, 8 aa, Artificial Sequence
  Synthetic
HHAHDAYL

SEQ ID NO: 7 - RNA, 76 nt, Artificial Sequence
  Synthetic
guuuuagagcuagaaauagcaaguuaaaauaaggcuaguccguuaucaacuugaaaaaguggcaccgagucggugc

SEQ ID NO: 8 - RNA, 86 nt, Artificial Sequence
  Synthetic
guuuaagagcuaugcuggaaacagcauagcaaguuuaaauaaggcuaguccguuaucaacuugaaaaaguggcaccgagucggugc

SEQ ID NO: 9 - RNA, 86 nt, Artificial Sequence
  Synthetic
guuucagagcuaugcuggaaacagcauagcaaguugaaauaaggcuaguccguuaucaacuugaaaaaguggcaccgagucggugc

SEQ ID NO: 10 - RNA, 86 nt, Artificial Sequence; identical to SEQ ID NO: 8
  Synthetic

SEQ ID NO: 11 - RNA, 76 nt, Artificial Sequence; identical to SEQ ID NO: 7
  Synthetic

SEQ ID NO: 12 - RNA, 76 nt, Artificial Sequence; identical to SEQ ID NO: 7
  Synthetic

SEQ ID NO: 13 - RNA, 107 nt, Artificial Sequence
  Synthetic
guuguagcucccuuucucgaaaagagaaccguugcuacaauaaggccgucugaaaagaugugccgcaacgcucugccccuuaaagcuucugcuuuaaggggcuuuuu

SEQ ID NO: 14 - RNA, 121 nt, Artificial Sequence
  Synthetic
guuguagcucccuuucucauuucggaaacgaaaugagaaccguugcuacaauaaggccgucugaaaagaugugccgcaacgcucugccccuuaaagcuucugcuuuaaggggcaucguuua

SEQ ID NO: 15 - RNA, 121 nt, Artificial Sequence; identical to SEQ ID NO: 14
  Synthetic

SEQ ID NO: 16 - RNA, 93 nt, Artificial Sequence
  Synthetic
guuuuuguacucucaagauucaauaaucuugcagaagcuacaaagauaaggcuucaugccgaaaucaacacccugucauuuuauggcagggug

SEQ ID NO: 17 - RNA, 93 nt, Artificial Sequence; identical to SEQ ID NO: 16
  Synthetic

SEQ ID NO: 18 - RNA, 87 nt, Artificial Sequence
  Synthetic
guuuuuguacucucagaaaugcagaagcuacaaagauaaggcuucaugccgaaaucaacacccugucauuuuauggcaggguguuuu

SEQ ID NO: 19 - RNA, 99 nt, Artificial Sequence
  Synthetic
gucuuuguacucucaagauuuaagaaauuaaaucuugcagaagcuacaaagauaaggcuucaugccgaaaucaacacccugucauucuauggcagggug

SEQ ID NO: 20 - RNA, 125 nt, Artificial Sequence
  Synthetic
guuuuuguacucucaagauuuaaguaacuguacaacgaaacuuacacaguuacuuaaaucuugcagaagcuacaaagauaaggcuucaugccgaaaucaacacccugucauuuuauggcagggug

SEQ ID NO: 21 - RNA, 80 nt, Artificial Sequence
  Synthetic
guuuuuguacucugguaccagaagcuacaaagauaaggcuucaugccgaaaucaacacccugucauuuuauggcagggug

SEQ ID NO: 22 - RNA, 76 nt, Artificial Sequence
  Synthetic
guuuuuguacucgaaagaagcuacaaagauaaggcuucaugccgaaaucaacacccugucauuuuauggcagggug

SEQ ID NO: 23 - RNA, 92 nt, Artificial Sequence
  Synthetic
guuuuagagcuguguuguuuguuaaaacaacacagcgaguuaaaauaaggcuuaguccguacucaacuugaaaagguggcaccgauucggug

SEQ ID NO: 24 - RNA, 94 nt, Artificial Sequence
  Synthetic
guuuuagagcuguguuguuucgguuaaaacaacacagcgaguuaaaauaaggcuuaguccguacucaacuugaaaagguggcaccgauucggug

SEQ ID NO: 25 - RNA, 78 nt, Artificial Sequence
  Synthetic
guuuuagagcuggguacccagcgaguuaaaauaaggcuuaguccguacucaacuugaaaagguggcaccgauucggug

SEQ ID NO: 26 - RNA, 76 nt, Artificial Sequence
  Synthetic
guuuuaguacucuggaaacagaaucuacuaaaacaaggcaaaaugccguguuuaucucgucaacuuguuggcgaga

SEQ ID NO: 27 - RNA, 84 nt, Artificial Sequence
  Synthetic
guuuuaguacucuguaaugaaaauuacagaaucuacuaaaacaaggcaaaaugccguguuuaucucgucaacuuguuggcgaga

SEQ ID NO: 28 - RNA, 124 nt, Artificial Sequence
  Synthetic
guuuuaguacucuguaauuuuagguaugagguagacgaaaauuguacuuauaccuaaaauuacagaaucuacuaaaacaaggcaaaaugccguguuuaucucgucaacuuguuggcgagauccg

SEQ ID NO: 29 - RNA, 133 nt, Artificial Sequence
  Synthetic
guuugagaguuguguaauuuaagauggaucucaaagaaauuuaagauccaucuuaaauuacacaacgaguucaaauaagaauucaucaaaaucgucccuuuugggaccgcucauuguggagcaucuuuuuuuu

SEQ ID NO: 30 - RNA, 133 nt, Artificial Sequence
  Synthetic
guuugagaguuguguaauuuaagauggaucucaaagaaauuugagauccaucuuaaauuacacaacgaguucaaauaagaauucaucaaaaucgucccuuuugggaccgcucauuguggagcaucuuuuuuuu

SEQ ID NO: 31 - RNA, 109 nt, Artificial Sequence
  Synthetic
guuugagaguuguguaauuuaaggaaacuuaaauuacacaacgaguucaaauaagaauucaucaaaaucgucccuuuugggaccgcucauuguggagcaucuuuuuuuu

SEQ ID NO: 32 - RNA, 20 nt, Artificial Sequence
  Synthetic
  misc_feature (11)..(11): n can be absent or C, A, or G
  misc_feature (12)..(12): n can be absent or G, A, or U
  misc_feature (13)..(13): n can be absent or G or U
aauuucuacunnnuguagau

SEQ ID NO: 33 - RNA, 19 nt, Artificial Sequence
  Synthetic
aauuucuacuguuguagau

SEQ ID NO: 34 - RNA, 19 nt, Artificial Sequence
  Synthetic
aauuucuacucuuguagau

SEQ ID NO: 35 - RNA, 20 nt, Artificial Sequence
  Synthetic
aauuucuacuaaguguagau

SEQ ID NO: 36 - RNA, 19 nt, Artificial Sequence
  Synthetic
aauuucuacuguuguagau

SEQ ID NO: 37 - RNA, 19 nt, Artificial Sequence
  Synthetic
aauuucuacuauuguagau

SEQ ID NO: 38 - RNA, 19 nt, Artificial Sequence
  Synthetic
aauuucuacuauuguagau

SEQ ID NO: 39 - RNA, 19 nt, Artificial Sequence
  Synthetic
aauuucuacuuuuguagau

SEQ ID NO: 40 - RNA, 20 nt, Artificial Sequence
  Synthetic
aauuucuacuguuuguagau

SEQ ID NO: 41 - RNA, 20 nt, Artificial Sequence
  Synthetic
aauuucuacuguuuguagau

SEQ ID NO: 42 - RNA, 20 nt, Artificial Sequence
  Synthetic
aauuucuacuguuuguagau

SEQ ID NO: 43 - RNA, 19 nt, Artificial Sequence
  Synthetic
aauuucuacuguuguagau

SEQ ID NO: 44 - RNA, 19 nt, Artificial Sequence
  Synthetic
aauuucuacuauuguagau

SEQ ID NO: 45 - RNA, 19 nt, Artificial Sequence
  Synthetic
aauuucuacuauuguagau

SEQ ID NO: 46 - RNA, 19 nt, Artificial Sequence
  Synthetic
aauuucuacuuuuguagau

SEQ ID NO: 47 - RNA, 19 nt, Artificial Sequence
  Synthetic
gauuucuacuuuuguagau

SEQ ID NO: 48 - RNA, 19 nt, Artificial Sequence
  Synthetic
aauuucuacuaguguagau

SEQ ID NO: 49 - RNA, 19 nt, Artificial Sequence
  Synthetic
aauuucuacuauuguagau

SEQ ID NO: 50 - RNA, 19 nt, Artificial Sequence
  Synthetic
aauuucuacuguuguagau

SEQ ID NO: 51 - RNA, 19 nt, Artificial Sequence
  Synthetic
aauuucuacuauuguagau

SEQ ID NO: 52 - protein, 12 aa, Artificial Sequence
  Synthetic
RRQRRTSKLMKR

SEQ ID NO: 53 - protein, 27 aa, Artificial Sequence
  Synthetic
GWTLNSAGYLLGKINLKALAALAKKIL

SEQ ID NO: 54 - protein, 33 aa, Artificial Sequence
  Synthetic
KALAWEAKLAKALAKALAKHLAKALAKALKCEA

SEQ ID NO: 55 - protein, 16 aa, Artificial Sequence
  Synthetic
RQIKIWFQNRRMKWKK

SEQ ID NO: 56 - protein, 11 aa, Artificial Sequence
  Synthetic
YGRKKRRQRRR

SEQ ID NO: 57 - protein, 9 aa, Artificial Sequence
  Synthetic
RKKRRQRRR

SEQ ID NO: 58 - protein, 11 aa, Artificial Sequence
  Synthetic
YGRKKRRQRRR

SEQ ID NO: 59 - protein, 8 aa, Artificial Sequence
  Synthetic
RKKRRQRR

SEQ ID NO: 60 - protein, 11 aa, Artificial Sequence
  Synthetic
YARAAARQARA

SEQ ID NO: 61 - protein, 11 aa, Artificial Sequence
  Synthetic
THRLPRRRRRR

SEQ ID NO: 62 - protein, 11 aa, Artificial Sequence
  Synthetic
GGRRARRRRRR

(19) United States
(12) Patent Application Publication
(10) Pub. No.: US 2020/0017852 A1
(43) Pub. Date: Jan. 16, 2020

(54) COMPOSITIONS AND METHODS FOR TARGET NUCLEIC ACID MODIFICATION

(71) Applicant: GenEdit Inc., Berkeley, CA (US)

(72) Inventor: Kunwoo LEE, Berkeley, CA (US)

(21) Appl. No.: 16/417,461

(22) Filed: May 20, 2019

Related U.S. Application Data
(63) Continuation-in-part of application No. PCT/US2017/062617, filed on Nov. 20, 2017.
(60) Provisional application No. 62/480,195, filed on Mar. 31, 2017.
(60) Provisional application No. 62/425,534, filed on Nov. 22, 2016.
(60) Provisional application No. 62/424,328, filed on Nov. 18, 2016.

(51) Int. Cl. C12N 15/11; C12N 9/22; C12N 15/90; C12N 15/10

(52) U.S. Cl. CPC C12N 15/11; C12N 9/22; C12N 2800/80; C12N 15/1034; C12N 2310/20; C12N 15/907

(57) ABSTRACT

The present disclosure provides methods and compositions utilizing CRISPR systems wherein the guide RNA and the donor polynucleotide are modified.

### CROSS-REFERENCE TO RELATED APPLICATIONS

This patent application claims priority to International (PCT) Patent Application PCT/US2017/062617 filed on Nov. 20, 2017, which claims priority to U.S. Provisional Patent Application No. 62/424,328, filed on Nov. 18, 2016; U.S. Provisional Patent Application No. 62/425,534, filed on Nov. 22, 2016; and U.S. Provisional Application No. 62/480,195, filed on Mar. 31, 2017, the entire disclosures of which are hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 37,945 Byte ASCII (Text) file named “512899_ST25.txt,” created on May 20, 2019.

### INTRODUCTION

RNA-mediated adaptive immune systems in bacteria and archaea rely on Clustered Regularly Interspaced Short Palindromic Repeat (CRISPR) genomic loci and CRISPR-associated (Cas) proteins that function together to provide protection from invading viruses and plasmids. In Type II CRISPR-Cas systems, the Cas9 protein functions as an RNA-guided endonuclease that uses a dual-guide RNA consisting of crRNA and trans-activating crRNA (tracrRNA) for target recognition and cleavage by a mechanism involving two nuclease active sites that together generate double-stranded DNA breaks (DSBs).

RNA-programmed Cas9 has proven to be a versatile tool for genome engineering in multiple cell types and organisms. Guided by a dual-RNA complex or a chimeric single-guide RNA, Cas9 (or variants of Cas9 such as nickase variants) can generate site-specific DSBs or single-stranded breaks (SSBs) within target nucleic acids. Target nucleic acids can include double-stranded DNA (dsDNA) and single-stranded DNA (ssDNA) as well as RNA. When cleavage of a target nucleic acid occurs within a cell (e.g., a eukaryotic cell), the break in the target nucleic acid can be repaired by non-homologous end joining (NHEJ) or homology directed repair (HDR). In addition, catalytically inactive Cas9 alone or fused to transcriptional activator or repressor domains can be used to alter transcription levels at sites within target nucleic acids by binding to the target site without cleavage.

Thus, the Cas9 system provides a facile means of modifying genomic information, and genome editing with Cas9-based therapeutics has the potential to treat a variety of previously incurable genetic diseases. Despite their considerable promise, however, Cas9-based therapeutics remain challenging due to the lack of effective delivery methods. Current approaches employing conventional viral delivery technologies can lead to toxicity from the viral vectors, as well as off-target genomic damage from sustained expression of Cas9. Accordingly, more effective and more targeted delivery techniques are still needed.

### SUMMARY

Provided herein are modified guide RNA and donor nucleic acid molecules and compositions, which are useful in conjunction with RNA-guided endonucleases (e.g., Cas9 or Cpf1) for gene editing, as well as CRISPR systems comprising such modified guide RNA and donor nucleic acid molecules. The present disclosure demonstrates that the 3′ and 5′ termini of guide RNA and donor polynucleotides are tolerant of variety of modifications without consequent loss of activity, and provides guide RNA and donor polynucleotides modified at the 3′ and/or 5′ ends as well as compositions and CRISPR systems comprising same and methods of using same, for instance, to edit genetic materials or screen for compounds that enhance the gene editing process.

According to one aspect of the disclosure, there is provided a guide RNA modified modified at the 3′ terminus or 5′ terminus with an amine, thiol, alkyne, strained alkyne, strained alkene, azide, or tetrazine group; modified at the 3′ or 5′ terminus with a detectable label or affinity tag (e.g., fluorescent molecule, biotin, etc.); or linked at the 3′ or 5′ terminus to the 3′ or 5′ end of another nucleic acid molecule, particularly a DNA molecule, such as a donor DNA. Also provided is a CRISPR system comprising such a modified guide RNA and a composition comprising the modified guide RNA.

According to another aspect of the disclosure, there is provided a donor polynucleotide modified at the 3′ or 5′ terminus with an amine, thiol, alkyne, strained alkyne, strained alkene, azide, or tetrazine group; or modified at the 3′ or 5′ terminus with a detectable label or affinity tag (e.g., fluorescent molecule, biotin, etc.). Also provided is a CRISPR system comprising such a modified donor polynucleotide, and a composition comprising the modified donor polynucleotide.

In another aspect, the disclosure provides a guide RNA linked to a donor polynucleotide, as well as a CRISPR system or complex comprising an RNA-guided endonuclease (e.g., a Cas9 or Cpf1 polypeptide), a guide RNA, and a donor polynucleotide, wherein the guide RNA is linked to the donor polynucleotide. As demonstrated herein, the guide RNA can be advantageously linked either covalently (e.g. via chemical or enzymatic ligation) or non-covalently (e.g. via hybridization) to the donor polynucleotide so as to enhance delivery efficiency and targeting. In particular, it is believed that linking the donor polynucleotide to the guide RNA enhances HDR by reducing the distance between the donor polynucleotide and the cleavage site. Additionally, the linked guide RNA and donor polynucleotide behaves like a single molecule, which can also increase delivery efficiency.

In a particular embodiment, the guide RNA comprises an extension sequence at the 3′ or 5′ end. Optionally, the extension sequence hybridizes to a region of the 3′ or 5′ end of a donor polynucleotide (e.g., a region of the donor polynucleotide that includes the 3′ or 5′ terminus). Optionally, the extension sequence contains multiple hybridization regions, which can be the same or different, allowing the guide RNA to hybridize to a region of the 3′ or 5′ end of multiple donor polynucleotides, which can be the same or different. In another embodiment, the guide RNA is linked to a donor RNA by way of a bridging polynucleotide, wherein the bridging polynucleotide hybridizes to both a region of the 3′ or 5′ end of the guide RNA and a region of the 3′ or 5′ end of the donor polynucleotide. Also provided is a CRISPR system comprising such a modified guide RNA and a composition comprising the modified guide RNA.

In particular embodiments, the CRISPR system or complex can be a Type II or Type V CRISPR system or complex. The present disclosure further provides also methods of making and using a complex of the present disclosure.

Remarkably, the 3′ and 5′ ends of the donor polynucleotide are also surprisingly tolerant of a wide variety of modifications (e.g., amine, azide, and fluorescent molecules). Accordingly, also provided herein are CRISPR systems comprising such modified donor polynucleotides. As such, multiple ways of linking the guide RNA to the donor polynucleotide are contemplated and enabled by the present invention.

Optionally, the inventive complexes further comprise a nanoparticle, as described in more detail in International Patent Application No. PCT/US2016/052690, the disclosure of which is expressly incorporated by reference herein. In some embodiments, the nanoparticle is a metal nanoparticle (e.g., a colloidal metal nanoparticle), such as a gold nanoparticle. In other embodiments, the nanoparticle is a polymer nanoparticle. In some embodiments, the nanoparticle has a diameter in the range of 10 nm to 1000 nm. In some embodiments, the nanoparticle has a diameter in the range of 5 nm to 150 nm. In some embodiments, the complex lacks a nanoparticle. In some embodiments, the complex of the subject invention is encapsulated in a suitable polymeric or liposomal system.

In some embodiments, the RNA-guided endonuclease is enzymatically active. In some embodiments, the RNA-guided endonuclease exhibits reduced enzymatic activity relative to a wild-type RNA-guided endonuclease, and wherein the subject RNA-guided endonuclease retains target nucleic acid binding activity. In some embodiments, the RNA-guided endonuclease comprises a nuclear localization signal. In some embodiments, the guide RNA is a single-molecule guide RNA. In some embodiments, the guide RNA is a dual-molecule guide RNA, e.g., crRNA and tracrRNA.

In another aspect, the present disclosure provides an encapsulated complex comprising: a) a CRISPR system (e.g. a Type II or a Type V CRISPR system) comprising: i) an RNA-guided endonuclease (e.g. a Cas9 or Cpf1 polypeptide); and ii) a guide RNA linked to a donor polynucleotide, wherein the complex is encapsulated in a suitable polymer or liposomal system, preferably a cationic polymer or liposomal system. In some embodiments, the encapsulated complex further comprises a silicate; for example, in some embodiments, the polymer and the silicate encapsulate the CRISPR system.

In some embodiments, the cationic polymer system comprises an endosomal disruptive polymer. In some embodiments, the endosomal disruptive polymer is a cationic polymer selected from the group consisting of polyethylene imine, poly(arginine), poly(lysine), poly(histidine), poly-[2-{(2-aminoethyl)amino}-ethyl-aspartamide] (pAsp(DET)), a block co-polymer of poly(ethylene glycol) (PEG) and poly(arginine), a block co-polymer of PEG and poly(lysine), and a block co-polymer of PEG and poly{N—[N-(2-aminoethyl)-2-aminoethyl]aspartamide} (PEG-pAsp(DET)). In some embodiments, the endosomal disruptive polymer is poly{N—[N-(2-aminoethyl)-2-aminoethyl]aspartamide} (pAsp(DET).

In some embodiments, the encapsulated complex further comprises a nanoparticle, e.g. a colloidal metal nanoparticle or polymer nanoparticle. In some embodiments, the nanoparticle is a gold nanoparticle. In some embodiments, the nanoparticle has a diameter in the range of 10 nm to 1000 nm. In some embodiments, the nanoparticle has a diameter in the range of 10 nm to 50 nm.

In some embodiments, the Cas9 or Cpf1 polypeptide is enzymatically active. In some embodiments, the Cas9 or Cpf1 polypeptide exhibits reduced enzymatic activity relative to a wild-type Cas9 or Cpf1 polypeptide, and wherein the Cas9 or Cpf1 polypeptide retains target nucleic acid binding activity. In some embodiments, the Cas9 or Cpf1 polypeptide comprises a nuclear localization signal. In some embodiments, the guide RNA is a single-molecule guide RNA. In some embodiments, the guide RNA is a dual-molecule guide RNA.

In another aspect, the invention provides a method of producing a complex comprising: contacting components of a CRISPR system (e.g. a Type II or a Type V CRISPR system) comprising: i) an RNA-guided endonuclease (e.g. a Cas9 or Cpf1 polypeptide) or nucleic acid (e.g., mRNA) encoding same; and ii) a guide RNA as provided herein, optionally linked to a donor polynucleotide or otherwise modified as described herein, to provide a complex; and ii) encapsulating the complex within one or more layers of an endosomal disruptive polymer. In some embodiments, the encapsulated complex further comprises a silicate; for example, in some embodiments, the polymer and the silicate encapsulate the CRISPR system.

The present disclosure provides a method of binding a target nucleic acid, comprising: contacting a cell comprising a target nucleic acid with a complex (e.g., an encapsulated complex) as described above or elsewhere herein, wherein the complex enters the cell, and wherein the RNA-guided endonuclease and guide RNA optionally linked to the donor polynucleotide are released from the complex in an endosome in the cell. In some embodiments, the cell is in vitro. In some embodiments, the cell is in vivo. In some embodiments, the RNA-guided endonuclease modulates transcription from the target nucleic acid. In some embodiments, the RNA-guided endonuclease modifies the target nucleic acid. In some embodiments, the RNA guided endonuclease cleaves the target nucleic acid. In the preferred embodiments contemplated herein, the complex (e.g., the encapsulated complex) comprises a donor polynucleotide, and the method comprises contacting the target nucleic acid with the donor polynucleotide. In particularly preferred embodiments, such contacting results in homology-directed repair.

The present disclosure provides a method of genetically modifying a target cell, comprising: contacting a target cell with a complex (e.g., an encapsulated complex) as described above or elsewhere herein. In some embodiments, the target cell is an in vivo target cell. In some embodiments, the target cell is a plant cell. In some embodiments, the target cell is an animal cell. In some embodiments, the target cell is a mammalian cell. In some embodiments, the target cell is a myoblast, a myofiber, a neuron, a chondrocyte, a lymphocyte, an epithelial cell, an adipocyte, a hematopoietic cell, or a keratinocyte. In some embodiments, the target cell is pluripotent cell.

Also provided is a method of screening for compounds that enhance gene editing using the modified guide RNA described herein. For instance, the guide RNA can be modified with an amine, thiol, alkyne, strained alkyne, strained alkene, azide, or tetrazine group. The method of screening for compounds that enhance the activity of an RNA-guided endonuclease can comprise: (a) linking a test compound to the modified guide RNA; combining (i) the guide RNA linked to the test compound; (ii) an RNA-guided endonuclease; (iii) a target DNA; and optionally (iv) a donor DNA; and (c) selecting the test compound as enhancing the activity of the RNA-guided endonuclease if the guide RNA linked to the test compound produces enhanced gene editing of the target DNA as compared to the guide RNA without the test compound.

The disclosure further provides a method of editing DNA in cells while enriching for cells most likely to be successfully edited, the method comprising: (a) administering an RNA guided endonuclease or nucleic acid (e.g., mRNA) encoding same, a guide RNA, and, optionally, donor nucleic acid to a cell comprising target DNA to be edited, wherein the guide RNA and/or donor nucleic acid, when present, comprises a detectable label; (b) selecting cells by detecting the detectable label; and (c) culturing the selected cells.

These and other aspects of the invention are provided herein.

### BRIEF DESCRIPTION OF THE DRAWINGS

FIG. 1 shows the amino acid sequence of Cas9 from Streptococcus pyogene (SEQ ID NO:1).

FIG. 2 shows the amino acid sequence of Cpf1 from Francisella tularensis subsp. novicida U112 (SEQ ID NO:2).

FIG. 3 illustrates the design of 3′ extended gRNAs. The figure shows non-extended gRNA, which has a size of about 102 nt, and four extended gRNAs with sequences of from about 120 to 140 nt (e.g., extension sequences of about 18 to about 38 nucleotides). gRNA_E1 has a sequence extended on the 3′ end that hybridizes the 3′ end of a donor DNA. gRNA_E2 has a sequence extended on the 3′ end that hybridizes the 5′ end of a donor DNA. gRNA_E3 has repeated sequence extensions that hybridize the 3′ ends of up to two donor DNAs. gRNA_E4 has a sequence extended on the 3′ end that hybridizes to a bridge nucleic acid, wherein the bride nucleic acid also hybridizes to the 5′ end of a donor DNA and connects gRNA_E4 and the donor DNA. Permutations of the illustrated designs (e.g., substituting 3′ extension or hybridization with 5′ extension or hybridization) will be apparent to the skilled person, and are encompassed by the invention.

FIG. 4 shows a gel electrophoretic separation of extended gRNAs hybridized to Donor DNA. Donor-hybridized gRNAs (gRNA_E1, gRNA_E2, and gRNA_E3 of FIG. 3) that are purified with 300 kDa concentrator show a clear band shift. In FIG. 4, E1/Donor corresponds to gRNA_E1 hybridized with Donor DNA, and similar nomenclature is used for the E2 and E3 guide/donor hybrids).

FIG. 5 provides the results of flow cytometry of BFP-HEK cells treated with Cas9 and extended gRNA/Donor DNAs.

FIG. 6 panels (a) and (b) illustrate synthetic schemes for chemical conjugation of modified crRNA and Donor DNA. The illustrated method also can be used with single guide RNA.

FIG. 7 is a graph of NHEJ frequency in BFP-K562 cells that are transfected with crRNA and crRNA-Donor DNA conjugates. 5′ and 3′ crRNA-Donor DNA conjugates were delivered together with tracrRNA and Cas9 protein and caused BFP knock-out in BFP-K562 cells.

FIG. 8 provides flow cytometry analysis of GFP population generation via Cas9 mediated homology directed repair (HDR), which shows efficient HDR with crRNA-Donor conjugates.

FIG. 9 illustrates a synthetic scheme for chemical conjugation of crRNA (Cpf1) and DNA.

FIG. 10 is a gel electrophoretic separation confirming the formation of crRNA-Donor DNA conjugate. Each band representing crRNA, Donor DNA, and crRNA-Donor DNA are marked with arrows.

FIG. 11 is a gel electrophoretic separation confirming Cpf1 activity of chemically modified Cpf1 crRNAs. 5′ amine and 5′ DBCO modified crRNAs showed levels of Cpf1 activity similar to that of unmodified crRNA during the in vitro cleavage assay. 5′ DNA modified crRNA showed reduced Cpf1 activity. Asterisk shows 5′ DNA modified crRNA band. Cleavage product has 350 bp size.

FIG. 12 is a graph of NHEJ frequency for Cpf1 crRNA-donor conjugate (DonorNA) transfected into GFP-HEK cells. Transfection of the cells with crRNA, donor, and Cpf1 without conjugation of the crRNA and donor nucleic acid served as a control.

FIG. 13 is a graph of HDR frequency for Cpf1 crRNA-donor conjugate (DonorNA) transfected into GFP-HEK cells. Transfection of the cells with crRNA, donor, and Cpf1 without conjugation of the crRNA and donor nucleic acid served as a control.

FIG. 14 is an illustration depicting a general scheme of gRNA and Donor DNA enzymatic ligation using a bridge DNA.

FIG. 15 is a gel electrophoretic separation confirming the ligation of crRNA and Donor DNA.

FIG. 16 is a gel electrophoretic separation confirming the results of an in vitro cleavage assay using crRNA-Donor enzymatic ligate.

FIG. 17 is an illustration of a general scheme for rolling circle RNA synthesis. (Image Source: Zheng et al. Chem. Commun., 2014, 50, 2100-2103.)

FIG. 18 is a graph of yellow fluorescent protein (YFP) knock-out frequency for YFP-targeted Cas9 gRNA and long-gRNA (IgRNA) with Cas9 in YFP-HEK cells.

FIG. 19A provides the chemical structure of modified gRNAs, wherein DNA-crRNAs are crRNAs conjugated to 127nt scramble DNA oligonucleotide. Any of the illustrated modifications also can be utilized with single guide RNA.

FIG. 19B is a graph showing the activity of Cas9 crRNAs with 5′ or 3′ modifications electroporated into BFP-HEK cells, which activity is quantified based on NHEJ frequency analyzed by one way ANOVA, post-hoc Tukey test, significant difference from control, *, P<0.05, **, P<0.01.

FIG. 19C shows the activity of Cpf1 crRNAs with 5′ or 3′ modifications electroporated into BFP-HEK cells, which activity is quantified based on NHEJ frequency.

FIG. 19D provides the chemical structures of modified donor DNA.

FIG. 19E shows the activity of donor DNA with 5′ or 3′ modifications electroporated into BFP-HEK cells, which activity is quantified based on the ability to induce HDR.

FIG. 20A provides a schematic overview of a cell enrichment process by which cells are transfected with labeled-donor DNA, and sorted by flow cytometry.

FIG. 20B provides fluorescence and bright field images and graphical analysis of sorted cells with low levels of Alexa647 and high levels of Alexa647.

FIGS. 20C, 20D, and 20E shows Alexa647 based FACS sorting of BFP-HEK cells (FIG. 20C), BFP-K562 cells (FIG. 20D), and primary myoblasts (FIG. 20E) to enrich for cells that have a high probability of being edited via HDR (analyzed by one way ANOVA, post-hoc Tukey test, significant difference from control, *, P<0.05, **, P<0.01).

FIG. 21A is a schematic overview of gene editing with gDonor/Cas9 complexes in cells.

FIG. 21B is a gel electrophoretic separation confirming synthesis of gRNA-donor conjugated via click chemistry.

FIG. 21C is a graph of HDR frequency in BFP-HEK cells for non-conjugated gRNA and gRNA-donor DNA (“gDonor”) conjugated via click chemistry.

FIG. 21D is a graph of NHEJ frequency BFP-HEK for gRNA-donor DNA conjugated via click chemistry showing a dose-dependent response.

FIG. 21E is a deep sequencing analysis of BFP-HEK cells edited with gDonor/Cas9 and comparison to cells edited with Cas9 RNP and donor DNA (control), showing that Cas9 with gDonor has an almost identical DNA cleavage profile as the unmodified control. The targeted Cas9 cleavage site for these experiments was at 64 locus (position of mutation), which is where most of the mutations were observed.

FIG. 21F is a graph of HDR frequency for gDonor/Cas9 complexes delivered into cells with cationic polymers compared to cationic polymers complexed to unconjugated gRNA and donor DNA. gDonor/Cas9 complexed to pAsp(DET) was three times more efficient at generating HDR in BFP-HEK cells than pAsp(DET) complexed to Cas9 RNP and donor DNA. An additional control composed of a scrambled DNA conjugated to the gRNA did not increase the transfection efficiency of pAsp(DET). Student-t-test, significant difference from gDonor/Cas9, **p<0.01.

FIG. 22 is a comparison of the protein-binding segments of Cpf1 crRNA sequences, with self-hybridizing right and left stem sequences identified. The sequences identified are Cpf1 crRNA from Lachnospiraceae bacterium ND2006 (LbCpf1), Candidatus Methanomethylophilus alvus Mx1201 (CMaCpf1), Sneatia amnii (SaCpf1), Acidaminococcus sp. BV3L6 (AsCpf1), Parcubacteria group bacterium GW2011 (PgCpf1); Candidatus Roizmanbacteria bacterium GW2011 (CRbCpf1), Candidatus Peregrinbacterium bacterium GW2011 (CPbCpf1), Lachnospiracea bacterium MA2020 (Lb5Cpf1), Btyrivibrio sp. NC3005 (BsCpf1), Butyrivibrio fibrisolvens (BfCpf1), Prevotella bryantii B14 (Pb2Cpf1), Bacteroidetes oral taxon 274 (BoCpf1), Flavobacterium brachiophilum FL-15 (FbCpf1), Lachnospiraceae bacterium MC2017 (Lb4Cpf1), Moraxella lacunata (MICpf1), Moraxella bovoculi AAX08_00205 (Mb2Cpf1), Moraxella bovoculi AAX11_00205 (Mb3Cpf1), Francisella novicida 0112 (FnCpf1) Thiomicrospira sp. XS5 (TsCpf1).

FIG. 23 is reaction scheme illustrating the preparation of DBCO-modified sgRNA according to Example 9.

### DEFINITIONS

The terms “polynucleotide” and “nucleic acid,” used interchangeably herein, refer to a polymer of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), and for hybridization of a DNA molecule with an RNA molecule: guanine (G) can also base pair with uracil (U). For example, G/U base-pairing is partially responsible for the degeneracy (i.e., redundancy) of the genetic code in the context of tRNA anti-codon base-pairing with codons in mRNA. Thus, in the context of this disclosure, a guanine (G) (e.g., of a protein-binding segment (dsRNA duplex) of a guide nucleic acid molecule; of a target nucleic acid base pairing with a guide nucleic acid, etc.) is considered complementary to both a uracil (U) and to an adenine (A). For example, when a G/U base-pair can be made at a given nucleotide position of a protein-binding segment (e.g., dsRNA duplex) of a subject guide nucleic acid molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. For hybridizations between nucleic acids with short stretches of complementarity (e.g. complementarity over 35 or less, 30 or less, 25 or less, 22 or less, 20 or less, or 18 or less nucleotides) the position of mismatches can become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). The temperature and wash solution salt concentration may be adjusted as necessary according to factors such as length of the region of complementation and the degree of complementation.

It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. In this example, the remaining non-complementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Exemplary methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g. with reference to an RNA-binding domain of a polypeptide, binding to a target nucleic acid, and the like) refers to a non-covalent interaction between macromolecules (e.g., between a protein and a nucleic acid; between a subject Cas9/guide nucleic acid complex and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{d}) of less than 10⁻⁶ M, less than 10⁻⁷ M, less than 10⁻⁶ M, less than 10⁻⁹ M, less than 10⁻¹⁹ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{d}.

By “binding domain” it is meant a protein domain that is able to bind non-covalently to another molecule. A binding domain can bind to, for example, a DNA molecule (a DNA-binding domain), an RNA molecule (an RNA-binding domain) and/or a protein molecule (a protein-binding domain). In the case of a protein having a protein-binding domain, it can in some embodiments bind to itself (to form homodimers, homotrimers, etc.) and/or it can bind to one or more regions of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Bioi. 215:403-10.

A DNA sequence that “encodes” a particular RNA is a DNA nucleic acid sequence that is transcribed into RNA. A DNA polynucleotide may encode an RNA (mRNA) that is translated into protein, or a DNA polynucleotide may encode an RNA that is not translated into protein (e.g. tRNA, rRNA, microRNA (miRNA), a “non-coding” RNA (ncRNA), a guide nucleic acid, etc.).

A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

The term “naturally-occurring” or “unmodified” or “wild type” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is wild type (and naturally occurring).

“Heterologous,” as used herein, means a nucleotide or polypeptide sequence that is not found in the native nucleic acid or protein, respectively. For example, in a chimeric Cas9 protein, the RNA-binding domain of a naturally-occurring bacterial Cas9 polypeptide (or a variant thereof) may be fused to a heterologous polypeptide sequence (i.e. a polypeptide sequence from a protein other than Cas9 or a polypeptide sequence from another organism). The heterologous polypeptide sequence may exhibit an activity (e.g., enzymatic activity) that will also be exhibited by the chimeric Cas9 protein (e.g., methyltransferase activity, acetyltransferase activity, kinase activity, ubiquitinating activity, etc.). A heterologous nucleic acid sequence may be linked to a naturally-occurring nucleic acid sequence (or a variant thereof) (e.g., by genetic engineering) to generate a chimeric nucleotide sequence encoding a chimeric polypeptide. As another example, in a fusion variant Cas9 polypeptide, a variant Cas9 polypeptide may be fused to a heterologous polypeptide (i.e. a polypeptide other than Cas9), which exhibits an activity that will also be exhibited by the fusion variant Cas9 polypeptide. A heterologous nucleic acid sequence may be linked to a variant Cas9 polypeptide (e.g., by genetic engineering) to generate a nucleotide sequence encoding a fusion variant polypeptide.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below). Alternatively, DNA sequences encoding RNA (e.g., guide nucleic acid) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

A cell has been “genetically modified” or “transformed” or “transfected” by exogenous DNA, e.g. a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

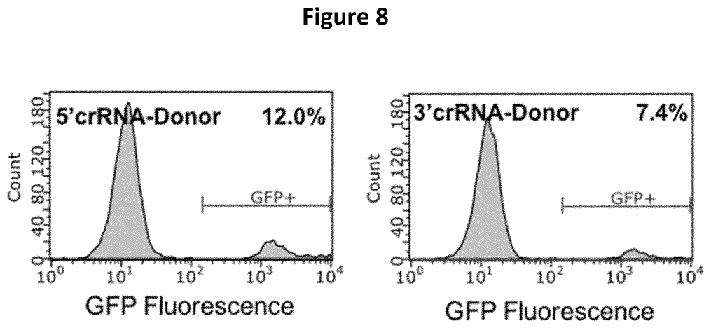

A “target nucleic acid” as used herein is a polynucleotide (e.g., RNA, DNA) that includes a “target site” or “target sequence.” The terms “target site” or “target sequence” are used interchangeably herein to refer to a nucleic acid sequence present in a target nucleic acid to which a targeting segment of a subject guide nucleic acid will bind (see FIG. 8), provided sufficient conditions for binding exist. For example, the target site (or target sequence) 5′-GAGCAUAUC-3′ within a target nucleic acid is targeted by (or is bound by, or hybridizes with, or is complementary to) the sequence 5′-GAUAUGCUC-3′. Suitable hybridization conditions include physiological conditions normally present in a cell. For a double stranded target nucleic acid, the strand of the target nucleic acid that is complementary to and hybridizes with the guide nucleic acid is referred to as the “complementary strand”; while the strand of the target nucleic acid that is complementary to the “complementary strand” (and is therefore not complementary to the guide nucleic acid) is referred to as the “noncomplementary strand” or “non-complementary strand”. In embodiments where the target nucleic acid is a single stranded target nucleic acid (e.g., single stranded DNA (ssDNA), single stranded RNA (ssRNA)), the guide nucleic acid is complementary to and hybridizes with single stranded target nucleic acid.

By “RNA-guided endonuclease polypeptide” or “RNA-guided endonuclease” it is meant a polypeptide that binds RNA (e.g., the protein binding segment of a guide nucleic acid) and is targeted to a specific sequence (a target site) in a target nucleic acid. For example, a Cas9 polypeptide or Cpf1 polypeptide as described herein is targeted to a target site by the guide nucleic acid to which it is bound. The guide nucleic acid comprises a sequence that is complementary to a target sequence within the target nucleic acid, thus targeting the bound Cas9 or Cpf1 polypeptide to a specific location within the target nucleic acid (the target sequence) (e.g., stabilizing the interaction of Cas9 or Cpf1 with the target nucleic acid). In some embodiments, the Cas9 or Cpf1 polypeptide is a naturally-occurring polypeptide (e.g., naturally occurs in bacterial and/or archaeal cells). In other embodiments, the Cas9 or Cpf1 polypeptide is not a naturally-occurring polypeptide (e.g., the Cas9 or Cpf1 polypeptide is a variant polypeptide, a chimeric polypeptide as discussed below, and the like).

Naturally occurring Cas9 and Cpf1 polypeptides bind a guide nucleic acid, are thereby directed to a specific sequence within a target nucleic acid (a target site), and cleave the target nucleic acid (e.g., cleave dsDNA to generate a double strand break, cleave ssDNA, cleave ssRNA, etc.). A subject Cas9 or Cpf1 polypeptide comprises two portions, an RNA-binding portion and an activity portion. An RNA-binding portion interacts with a subject guide nucleic acid. An activity portion exhibits site-directed enzymatic activity (e.g., nuclease activity, activity for DNA and/or RNA methylation, activity for DNA and/or RNA cleavage, activity for histone acetylation, activity for histone methylation, activity for RNA modification, activity for RNA-binding, activity for RNA splicing etc.). In some embodiments, the activity portion exhibits reduced nuclease activity relative to the corresponding portion of a wild type Cas9 or Cpf1 polypeptide. In some embodiments, the activity portion is enzymatically inactive.

By “cleavage” it is meant the breakage of the covalent backbone of a target nucleic acid molecule (e.g., RNA, DNA). Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. In certain embodiments, a complex comprising a guide nucleic acid and a Cas9 or Cpf1 polypeptide is used for targeted cleavage of a single stranded target nucleic acid (e.g., ssRNA, ssDNA).

“Nuclease” and “endonuclease” are used interchangeably herein to mean an enzyme which possesses catalytic activity for nucleic acid cleavage (e.g., ribonuclease activity (ribonucleic acid cleavage), deoxyribonuclease activity (deoxyribonucleic acid cleavage), etc.).

By “cleavage domain” or “active domain” or “nuclease domain” of a nuclease it is meant the polypeptide sequence or domain within the nuclease which possesses the catalytic activity for nucleic acid cleavage. A cleavage domain can be contained in a single polypeptide chain or cleavage activity can result from the association of two (or more) polypeptides. A single nuclease domain may consist of more than one isolated stretch of amino acids within a given polypeptide.

A nucleic acid molecule that binds to the RNA-guided endonuclease and targets the polypeptide to a specific location within the target nucleic acid is referred to herein as a “guide nucleic acid”. When the guide nucleic acid comprises RNA, it can be referred to as a “guide RNA” or a “gRNA”. A guide nucleic acid comprises two segments, a first segment (referred to herein as a “targeting segment”); and a second segment (referred to herein as a “protein-binding segment”). By “segment” it is meant a segment/section/region of a molecule, e.g., a contiguous stretch of nucleotides in a nucleic acid molecule. A segment can also mean a region/section of a complex such that a segment may comprise regions of more than one molecule. For example, in some embodiments the protein-binding segment (described below) of a guide nucleic acid is one nucleic acid molecule (e.g., one RNA molecule) and the protein-binding segment therefore comprises a region of that one molecule. In other embodiments, the protein-binding segment (described below) of a guide nucleic acid comprises two separate molecules that are hybridized along a region of complementarity. As an illustrative, non-limiting example, a protein-binding segment of a guide nucleic acid that comprises two separate molecules might comprise (i) base pairs 40-75 of a first molecule (e.g., RNA molecule or DNA/RNA hybrid molecule) that is approximately 100 base pairs in length; or (ii) base pairs 10-25 of a second molecule (e.g., RNA molecule) that is 50 base pairs in length. The definition of “segment,” unless otherwise specifically defined in a particular context, is not limited to a specific number of total base pairs, is not limited to any particular number of base pairs from a given nucleic acid molecule, is not limited to a particular number of separate molecules within a complex, and may include regions of nucleic acid molecules that are of any total length and may or may not include regions with complementarity to other molecules.

The first segment (targeting segment) of a guide nucleic acid (e.g., guide RNA) comprises a nucleotide sequence that is complementary to a specific sequence (a target site) within a target nucleic acid to be edited (e.g., a target ssRNA, a target ssDNA, the complementary strand of a double stranded target DNA, etc.). The protein-binding segment (or “protein-binding sequence”) interacts with an RNA guided endonuclease (e.g., a Cas9 or Cpf1 polypeptide). Site-specific binding and/or cleavage of the target nucleic acid can occur at locations determined by base-pairing complementarity between the guide nucleic acid (e.g., guide RNA) and the target nucleic acid.

The protein-binding segment of a guide nucleic acid comprises at least two complementary stretches of nucleotides (i.e., at least one pair of self-hybridizing sequences) that hybridize to one another to form a double stranded RNA duplex (dsRNA duplex).

In some embodiments, a subject nucleic acid (e.g., a guide nucleic acid, a nucleic acid comprising a nucleotide sequence encoding a guide nucleic acid; a nucleic acid encoding a Cas9 polypeptide; etc.) comprises a modification or sequence (e.g., an additional segment at the 5′ and/or 3′ end) that provides for an additional desirable feature (e.g., modified or regulated stability; subcellular targeting; tracking, e.g., a fluorescent label; a binding site for a protein or protein complex; etc.). Non-limiting examples include: a 5′ cap (e.g., a 7-methylguanylate cap (m7G)); a 3′ polyadenylated tail (i.e., a 3′ poly(A) tail); a ribozyme sequence (e.g. to allow for self-cleavage and release of a mature molecule in a regulated fashion); a riboswitch sequence (e.g., to allow for regulated stability and/or regulated accessibility by proteins and/or protein complexes); a stability control sequence; a sequence that forms a dsRNA duplex (i.e., a hairpin)); a modification or sequence that targets the nucleic acid to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence such as a nucleic acid “barcode” that allows for tracking and detection, etc.); a modification or sequence that provides a binding site for proteins (e.g., proteins that act on DNA and/or RNA, including transcriptional activators, transcriptional repressors, DNA methyltransferases, DNA demethylases, histone acetyltransferases, histone deacetylases, and the like); and combinations thereof. In some embodiments, the subject nucleic acid comprises a nucleic acid (DNA or RNA) sequence “barcode,” which is a short (e.g., about 5-100 nt, 5-75 nt, 5-50 nt, 5-40 nt, 5-25 nt, or 5-15 nt) sequence that is sufficiently unique as to allow the sequence to serve as a tag that can be detected by nucleic acid amplification (PCR) or other suitable methods). Specific methods for creating and using nucleic acid barcodes are known in the art (see, e.g., Dahlman et al., Proc Natl Acad Sci USA.; 2017; 114(8): 2060-2065; Lyons et al., Scientific Reports, volume 7, article no. 13899 (2017)). The barcode can be attached to the guide nucleic acid or donor nucleic acid, or can be part of a linker linking a guide nucleic acid to a donor nucleic acid.

A subject guide nucleic acid (e.g., guide RNA) linked to a donor polynucleotide forms a complex with a subject RNA-guided endonuclease (i.e., binds via non-covalent interactions). The guide nucleic acid (e.g., guide RNA) provides target specificity to the complex by comprising a nucleotide sequence that is complementary to a sequence of a target nucleic acid. The RNA-guided endonuclease of the complex provides the site-specific activity. In other words, the RNA-guided endonuclease is guided to a target nucleic acid sequence (e.g. a target sequence in a chromosomal nucleic acid; a target sequence in an extrachromosomal nucleic acid, e.g. an episomal nucleic acid, a minicircle, an RNA, a DNA, etc.; a target sequence in a mitochondrial nucleic acid; a target sequence in a chloroplast nucleic acid; a target sequence in a plasmid; etc.) by virtue of its association with the protein-binding segment of the guide nucleic acid.

In some embodiments, a subject guide nucleic acid (e.g., guide RNA) comprises two separate nucleic acid molecules: an “activator” and a “targeter” (see below) and is referred to herein as a “dual guide nucleic acid”, a “double-molecule guide nucleic acid”, or a “two-molecule guide nucleic acid.” If both molecules of a dual guide nucleic acid are RNA molecules, the dual guide nucleic acid can be referred to as a “dual guide RNA” or a “dgRNA.”

When the guide RNA comprises two separate nucleic acid molecules, the two molecules each comprise a region or segment that is sufficiently complementary to the other to allow hybridization forming the dsRNA region referred to above. Thus, for instance, the targeter molecule comprises a targeting sequence that is complementary to a region of the target nucleic acid to be edited, and another sequence that hybridizes to a sequence of the activator molecule. The activator molecule, likewise, comprises the sequence that hybridizes to the targeter molecule and additional nucleotides as required for interaction with the RNA guided endonuclease protein. The dsRNA region formed by hybridization of a segment of the targeter molecule and a segment of the activator molecule interacts with the RNA guided endonuclease and is considered part of the protein-binding segment of the guide RNA.

In some embodiments, the subject guide nucleic acid is a single nucleic acid molecule (single polynucleotide) and is referred to herein as a “single guide nucleic acid”, a “single-molecule guide nucleic acid,” or a “one-molecule guide nucleic acid.” If a single guide nucleic acid is an RNA molecule, it can be referred to as a “single guide RNA” or an “sgRNA.” A single guide RNA includes a construct in which separate targeter and activator molecules are linked, such as by a linker sequence.

Thus, the term “guide nucleic acid” is inclusive, referring to both dual guide nucleic acids and to single guide nucleic acids (e.g., dgRNAs, sgRNAs, etc.) while the term “guide RNA” is also inclusive, referring to both dual guide RNA (dgRNA) and single guide RNA (sgRNA).

In some embodiments, a guide nucleic acid is a DNA/RNA hybrid molecule. In such embodiments, the protein-binding segment of the guide nucleic acid is RNA and forms an RNA duplex as described above. However, the targeting segment of a guide nucleic acid can be DNA. Thus, if a DNA/RNA hybrid guide nucleic acid is a dual guide nucleic acid, the “targeter” molecule and be a hybrid molecule (e.g, the targeting segment can be DNA and the duplex-forming segment can be RNA). In such embodiments, the duplex-forming segment of the “activator” molecule can be RNA (e.g., in order to form an RNA-duplex with the duplex-forming segment of the targeter molecule), while nucleotides of the “activator” molecule that are outside of the duplex-forming segment can be DNA (in which case the activator molecule is a hybrid DNA/RNA molecule) or can be RNA (in which case the activator molecule is RNA). If a DNA/RNA hybrid guide nucleic acid is a single guide nucleic acid, then the targeting segment can be DNA, the duplex-forming segments (which make up the protein-binding segment) can be RNA, and nucleotides outside of the targeting and duplex-forming segments can be RNA or DNA.

An exemplary dual guide nucleic acid comprises a crRNA-like (“CRISPR RNA” or “targeter” or “crRNA” or “crRNA repeat”) molecule and a corresponding tracrRNA-like (“trans-acting CRISPR RNA” or “activator” or “tracrRNA”) molecule. A crRNA-like molecule (targeter) comprises both the targeting segment (single stranded) of the guide nucleic acid and a stretch (“duplex-forming segment”) of nucleotides that forms one half of the dsRNA duplex of the protein-binding segment of the guide nucleic acid. A corresponding tracrRNA-like molecule (activator) comprises a stretch of nucleotides (duplex-forming segment) that forms the other half of the dsRNA duplex of the protein-binding segment of the guide nucleic acid. In other words, a stretch of nucleotides of a crRNA-like molecule are complementary to and hybridize with a stretch of nucleotides of a tracrRNA-like molecule to form the dsRNA duplex of the protein-binding domain of the guide nucleic acid. The crRNA-like molecule additionally provides the single stranded targeting segment. Thus, a crRNA-like and a tracrRNA-like molecule (as a corresponding pair) hybridize to form a dual guide nucleic acid.

An exemplary single guide nucleic acid (e.g., sgRNA) includes, for instance, a crRNA-like molecule (e.g., Cas9 crRNA) and a tracrRNA-like molecule (e.g., Cas9 tracrRNA) linked at the end of the dsRNA duplex by a linker nucleotide sequence. Another exemplary single guide RNA includes, for instance, a Cpf1 crRNA, which comprises a self-hybridizing dsRNA segment and provides both a protein binding segment and targeting segment.

The exact sequence of a given guide RNA (e.g., crRNA and/or tracrRNA) molecule is characteristic of the particular RNA guided endonuclease used. Many different RNA guided endonucleases are known in the art originating from many different species of microorganisms, each of which have corresponding RNA sequences in the protein binding segment of the guide RNA. The sequence of the targeting segment will, of course, depend on the particular sequence of the target nucleic acid to be edited. The guide RNA used in conjunction with the present invention is not limited to any particular guide RNA sequence, and finds utility with any guide RNA (e.g., any corresponding activator and targeter pair).

The term “activator” is used herein to refer to a tracrRNA-like molecule of a dual guide nucleic acid (and of a single guide nucleic acid when the “activator” and the “targeter” are linked together by intervening nucleic acids). The term “targeter” is used herein to refer to a crRNA-like molecule of a dual guide nucleic acid (and of a single guide nucleic acid when the “activator” and the “targeter” are linked together by intervening nucleic acids). The term “duplex-forming segment” is used herein to mean the stretch of nucleotides of an activator or a targeter that contributes to the formation of the dsRNA duplex by hybridizing to a stretch of nucleotides of a corresponding activator or targeter molecule. In other words, an activator comprises a duplex-forming segment that is complementary to the duplex-forming segment of the corresponding targeter. As such, an activator comprises a duplex-forming segment while a targeter comprises both a duplex-forming segment and the targeting segment of the guide nucleic acid. A subject single guide nucleic acid can comprise an “activator” and a “targeter” where the “activator” and the “targeter” are covalently linked (e.g., by intervening nucleotides). Therefore, a dual guide nucleic acid can be comprised of any corresponding activator and targeter pair.

A “host cell” or “target cell” as used herein, denotes an in vivo or in vitro eukaryotic cell, or a cell from a multicellular organism (e.g., a cell line) cultured as a unicellular entity, which eukaryotic cells can be, or have been, used as recipients for a nucleic acid, and include the progeny of the original cell which has been transformed by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a subject eukaryotic host cell is a genetically modified eukaryotic host cell, by virtue of introduction into a suitable eukaryotic host cell of an exogenous nucleic acid.

The terms “treatment”, “treating” and the like are used herein to generally mean obtaining a desired pharmacologic and/or physiologic effect. The effect may include inhibiting or reducing any effect or symptom of a disease or condition by any degree. The effect can be the alteration of a gene in a cell, optionally in a host, which, in turn, can have prophylactic or therapeutic effects in terms of completely or partially preventing a disease or symptom thereof and/or partially or completely inhibiting or reversing a disease and/or adverse effect (symptom) attributable to the disease. “Treatment” as used herein covers any treatment of a disease or symptom in a mammal. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some embodiments after the symptomatic stage of the disease.

The terms “individual,” “subject,” “host,” and “patient,” are used interchangeably herein and refer to any mammalian subject for whom diagnosis, treatment, or therapy is desired, particularly humans.

In some instances, a component (e.g., a nucleic acid component (e.g., a guide nucleic acid, etc.); a protein component (e.g., a Cas9 or Cpf1 polypeptide, a variant Cas9 or Cpf1 polypeptide); and the like) includes a label moiety. The terms “label”, “detectable label”, or “label moiety” as used herein refer to any moiety that provides for signal detection and may vary widely depending on the particular nature of the assay. Label moieties of interest include both directly detectable labels (direct labels)(e.g., a fluorescent label) and indirectly detectable labels (indirect labels)(e.g., a binding pair member). A fluorescent label can be any fluorescent label (e.g., a fluorescent dye (e.g., fluorescein, Texas red, rhodamine, ALEXAFLUOR® labels, and the like), a fluorescent protein (e.g., green fluorescent protein (GFP), enhanced GFP (EGFP), yellow fluorescent protein (YFP), red fluorescent protein (RFP), cyan fluorescent protein (CFP), cherry, tomato, tangerine, and any fluorescent derivative thereof), etc.). Suitable detectable (directly or indirectly) label moieties for use in the methods include any moiety that is detectable by spectroscopic, photochemical, biochemical, immunochemical, electrical, optical, chemical, or other means. For example, suitable indirect labels include biotin (a binding pair member), which can be bound by streptavidin (which can itself be directly or indirectly labeled). Labels can also include: a radiolabel (a direct label)(e.g., ³H, ¹²⁵I, ³⁵S, ¹⁴C, or ³²P); an enzyme (an indirect label)(e.g., peroxidase, alkaline phosphatase, galactosidase, luciferase, glucose oxidase, and the like); a fluorescent protein (a direct label)(e.g., green fluorescent protein, red fluorescent protein, yellow fluorescent protein, and any convenient derivatives thereof); a metal label (a direct label); a colorimetric label; a binding pair member; and the like. By “partner of a binding pair” or “binding pair member” is meant one of a first and a second moiety, wherein the first and the second moiety have a specific binding affinity for each other. Suitable binding pairs include, but are not limited to: antigen/antibodies (for example, digoxigenin/anti-digoxigenin, dinitrophenyl (DNP)/anti-DNP, dansyl-X-anti-dansyl, fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Any binding pair member can be suitable for use as an indirectly detectable label moiety.

Any given component, or combination of components can be unlabeled, or can be detectably labeled with a label moiety. In some embodiments, when two or more components are labeled, they can be labeled with label moieties that are distinguishable from one another.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a complex” includes a plurality of such complexes and reference to “the Cas9 polypeptide” includes reference to one or more Cas9 polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides modified components of a CRISPR system, as well as compositions comprising the modified CRISPR components and methods for the preparation and use thereof.

In one aspect, the invention provides a complex comprising a CRISPR system (e.g. a Type II or a Type V CRISPR system) comprising an RNA-guided endonuclease (e.g. a Cas9 or Cpf1 polypeptide) or nucleic acid encoding same, a guide nucleic acid and a donor polynucleotide, wherein the guide nucleic acid and the donor polynucleotide are linked or the guide nucleic and/or donor polynucleotide are otherwise modified as described herein. In one embodiment, the inventive complex comprises a Type II CRISPR system comprising a Cas9 polypeptide (or nucleic acid encoding same) and corresponding guide nucleic acid, and in other embodiments, the inventive complex comprises a Type V CRISPR system comprising a Cpf1 polypeptide (or nucleic acid encoding same) and corresponding guide RNA.

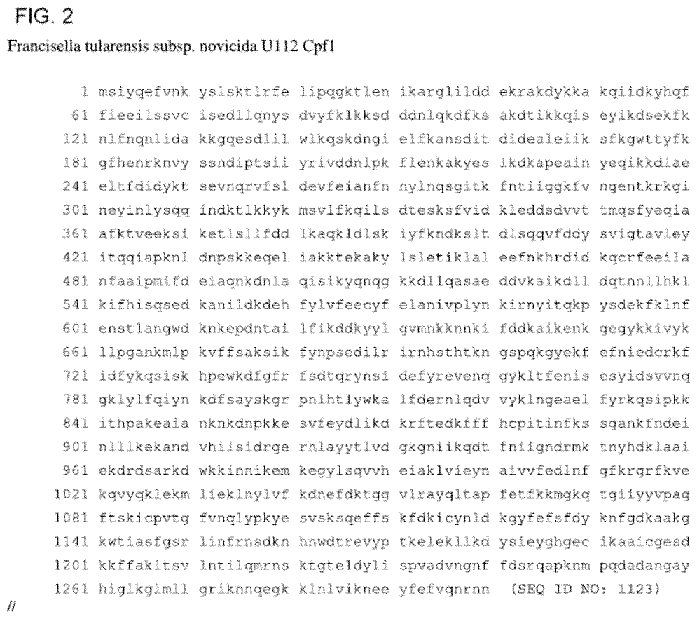

As exemplified herein, the guide nucleic acid and donor polynucleotide, which linked, can be either covalently or non-covalently linked. In one embodiment, the guide RNA and donor polynucleotide are chemically ligated. In another embodiment, the guide RNA and donor polynucleotide are enzymatically ligated. In still other embodiments, the guide RNA and donor polynucleotide hybridize to each other, or the guide RNA and donor polynucleotide both hybridize to a bridge sequence. Any number of such hybridization schemes are possible, including those illustrated in FIG. 2 and further exemplified herein.

In some embodiments, the complex of the subject invention is encapsulated in a suitable polymeric or liposomal system. In a particular embodiment, the complex is encapsulated in a polycation-based endosomal escape polymer.

### Donor Polynucleotide

Any suitable donor polynucleotide can be used in accordance with the invention (e.g., linked to a guide nucleic acid and/or otherwise modified as described herein). A “donor sequence,” “donor polynucleotide,” “donor nucleic acid,” or “donor DNA template” is a nucleic acid sequence to be inserted into a target nucleic acid at a cleavage site induced by an RNA-guided endonuclease (e.g., a Cas9 polypeptide or a Cpf1 polypeptide). The donor polynucleotide will contain sufficient homology (or sequence identity) to a target genomic sequence at the cleavage site, e.g. 70% or more, 80% or more, 85% or more, 90% or more, 95% or more, 99% or more, or even 100% percent identity with the nucleotide sequences flanking the cleavage site (e.g. within about 50 bases or less of the cleavage site, e.g. within about 30 bases, within about 15 bases, within about 10 bases, within about 5 bases, or immediately flanking the cleavage site), to support homology-directed repair between the donor nucleic acid and the genomic sequence to which it bears homology. Approximately 25, 50, 100, or 200 nucleotides, or more than 200 nucleotides, of sequence homology (or sequence identity) between a donor nucleic acid and a genomic sequence (or any integral value between 10 and 200 nucleotides, or more) will support homology-directed repair. Donor sequences can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain one or more single base changes (substitutions, insertions, deletions, inversions or rearrangements) as compared to the genomic sequence, so long as sufficient homology or sequence identity is present to facilitate homology-directed repair. In some embodiments, the donor sequence comprises a non-homologous sequence flanked by two regions of homology/sequence identity (homology “arms”), such that homology-directed repair between the target DNA region and the two flanking sequences results in insertion of the non-homologous sequence at the target region.

Donor sequences may also comprise or be part of a vector backbone containing sequences that are not homologous to the DNA region of interest and that are not intended for insertion into the DNA region of interest, such that only the donor sequence itself is inserted through homologous repair and the rest of the vector is not.

Generally, the homologous region(s) of a donor sequence (e.g., flanking a non-homologous region) will each have at least 70% sequence identity to a genomic sequence with which recombination is desired. In certain embodiments, 80% or more, 85% or more, 90% or more, 95% or or more, 98% or more, 99% or more, or even 99.9% or more sequence identity is present.

The donor sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some embodiments may be used for other purposes (e.g., to signify expression at the targeted genomic locus). In some embodiments, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

The donor sequence may be provided to the cell as single-stranded DNA, single-stranded RNA, double-stranded DNA, or double-stranded RNA. It may be introduced into a cell in linear or circular form. If introduced in linear form, the ends of the donor sequence may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad Sci USA 84:4959-4963; Nehls et al. (1996) Science 272:886-889. Amplification procedures such as rolling circle amplification can also be advantageously employed, as exemplified herein. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or polymer, or can be delivered by viruses (e.g., adenovirus, AAV), as described herein for nucleic acids encoding a Cas9 guide RNA and/or a Cas9 fusion polypeptide and/or donor polynucleotide.

The particular sequence of the donor nucleic acid is not limited, and will depend upon the sequence of the target nucleic acid to be edited. However, as a general matter, the donor nucleic acid sequence will be different from, and will not comprise, the sequence of the protein-binding segment of the guide RNA. Furthermore, the sequence of the donor nucleic acid typically will not comprise a sequence identical to the targeting sequence of the guide RNA. Typically, the donor sequence will differ from the target sequence by at least one nucleotide substitution, addition, or deletion, although the sequence of the donor nucleic acid might overlap with the targeting sequence and, therefore, can have regions that are identical to the target sequence.

### Guide RNA

Any suitable guide nucleic acid can be used in accordance with the invention (e.g., linked to a donor polynucleotide and/or otherwise modified as described herein). Guide nucleic acids suitable for inclusion in a complex of the present disclosure include any guide nucleic acid from any CRISPR system, including single-molecule guide nucleic acids (“single-guide RNA”/“sgRNA”) and dual-molecule guide nucleic acids (“dual-guide RNA”/“dgRNA”).

A guide nucleic acid (e.g., guide RNA) suitable for inclusion in a complex of the present disclosure directs the activities of an RNA-guided endonuclease (e.g., a Cas9 of Cpf1 polypeptide) to a specific target sequence within a target nucleic acid. A guide nucleic acid (e.g., guide RNA) comprises: a first segment (also referred to herein as a “nucleic acid targeting segment”, or simply a “targeting segment”); and a second segment (also referred to herein as a “protein-binding segment”). The terms “first” and “second” do not imply the order in which the segments occur in the guide RNA. The order of the elements relative to one another depends upon the particular RNA-guided polypeptide to be used. For instance, guide RNA for Cas9 typically has the protein-binding segment located 3′ of the targeting segment, whereas guide RNA for Cpf1 typically has the protein-binding segment located 5′ of the targeting segment.

The guide RNA may be introduced into a cell in linear or circular form. If introduced in linear form, the ends of the guide RNA may be protected (e.g., from exonucleolytic degradation) by methods known to those of skill in the art. Amplification procedures such as rolling circle amplification can also be advantageously employed, as exemplified herein.

First Segment: Targeting Segment

The first segment of a guide nucleic acid (e.g., guide RNA) includes a nucleotide sequence that is complementary to a sequence (a target site) in a target nucleic acid. In other words, the targeting segment of a guide nucleic acid (e.g., guide RNA) can interact with a target nucleic acid (e.g., an RNA, a DNA, a double-stranded DNA) in a sequence-specific manner via hybridization (i.e., base pairing). As such, the nucleotide sequence of the targeting segment may vary and can determine the location within the target nucleic acid that the guide nucleic acid (e.g., guide RNA) and the target nucleic acid will interact. The targeting segment of a guide nucleic acid (e.g., guide RNA) can be created/modified (e.g., by genetic engineering) to hybridize to any desired sequence (target site) within a target nucleic acid.

The targeting segment can have a length of from 12 nucleotides to 100 nucleotides. The nucleotide sequence (the targeting sequence, also referred to as a guide sequence) of the targeting segment that is complementary to a nucleotide sequence (target site) of the target nucleic acid can have a length of 12 nt or more. For example, the targeting sequence of the targeting segment that is complementary to a target site of the target nucleic acid can have a length of 12 nt or more, 15 nt or more, 17 nt or more, 18 nt or more, 19 nt or more, 20 nt or more, 25 nt or more, 30 nt or more, 35 nt or more or 40 nt.

The percent complementarity between the targeting sequence (i.e., guide sequence) of the targeting segment and the target site of the target nucleic acid can be 60% or more (e.g., 65% or more, 70% or more, 75% or more, 80% or more, 85% or more, 90% or more, 95% or more, 97% or more, 98% or more, 99% or more, or 100%). In some embodiments, the targeting sequence comprises a “seed” region of six or seven nucleotides that binds the region of target sequence closest the PAM site for the system being used, and the percent complementarity between the seed region of the targeting sequence of the targeting segment and the target site of the target nucleic acid is at least about 99%, 99.5%, or even 100% (e.g., at least about 99%, 99.5%, or even 100% complementarity over the six or seven contiguous 5′-most nucleotides of the target site of the target nucleic acid in the case of a Cas9 guide nucleic acid, or at least about 99%, 99.5%, or even 100% complementarity over the six or seven contiguous 3′-most nucleotides of the target site of the target nucleic acid in the case of a Cpf1 guide nucleic acid). In some embodiments, the percent complementarity between the targeting sequence of the targeting segment and the target site of the target nucleic acid is 60% or more over 20 contiguous nucleotides. In some embodiments, the percent complementarity between the targeting sequence of the targeting segment and the target site of the target nucleic acid is 100% over the seventeen, eighteen, nineteen or twenty contiguous 5′-most nucleotides of the target site of the target nucleic acid and as low as 0% or more over the remainder. In such a case, the targeting sequence can be considered to be 17, 18, 19 or 20 nucleotides in length, respectively.

Second Segment: Protein-Binding Segment

The protein-binding segment of a subject guide nucleic acid (e.g., guide RNA) interacts with (binds) an RNA-guided endonuclease. The subject guide nucleic acid (e.g., guide RNA) guides the bound endonuclease to a specific nucleotide sequence within target nucleic acid (the target site) via the above mentioned targeting segment/targeting sequence/guide sequence. The protein-binding segment of a subject guide nucleic acid (e.g., guide RNA) comprises two stretches of nucleotides that are complementary to one another. The complementary nucleotides of the protein-binding segment hybridize to form a double stranded RNA duplex (dsRNA).

A subject dual guide nucleic acid (e.g., guide RNA) comprises two separate nucleic acid molecules. Each of the two molecules of a subject dual guide nucleic acid (e.g., guide RNA) comprises a stretch of nucleotides that are complementary to one another such that the complementary nucleotides of the two molecules hybridize to form the double stranded RNA duplex of the protein-binding segment.

In some embodiments, the duplex-forming segment of the activator is 60%, 65%, 70%, 75%, 80%, 85%, 90%, 95%, 98%, 99% or more identical or 100% identical to one of the activator (tracrRNA) molecules set forth in International Patent Application No. PCT/US2016/052690, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

In some embodiments, the duplex-forming segment of the targeter is 60%, 65%, 70%, 75%, 80%, 85%, 90%, 95%, 98%, 99% or more identical or 100% identical to one of the targeter (crRNA) sequences set forth in International Patent Application No. PCT/US2016/052690, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

A dual guide nucleic acid (e.g., guide RNA) can be designed to allow for controlled (i.e., conditional) binding of a targeter with an activator. Because a dual guide nucleic acid (e.g., guide RNA) is not functional unless both the activator and the targeter are bound in a functional complex with Cas9, a dual guide nucleic acid (e.g., guide RNA) can be inducible (e.g., drug inducible) by rendering the binding between the activator and the targeter to be inducible. As one non-limiting example, RNA aptamers can be used to regulate (i.e., control) the binding of the activator with the targeter. Accordingly, the activator and/or the targeter can include an RNA aptamer sequence.

Aptamers (e.g., RNA aptamers) are known in the art and are generally a synthetic version of a riboswitch. The terms “RNA aptamer” and “riboswitch” are used interchangeably herein to encompass both synthetic and natural nucleic acid sequences that provide for inducible regulation of the structure (and therefore the availability of specific sequences) of the nucleic acid molecule (e.g., RNA, DNA/RNA hybrid, etc.) of which they are part. RNA aptamers usually comprise a sequence that folds into a particular structure (e.g., a hairpin), which specifically binds a particular drug (e.g., a small molecule). Binding of the drug causes a structural change in the folding of the RNA, which changes a feature of the nucleic acid of which the aptamer is a part. As non-limiting examples: (i) an activator with an aptamer may not be able to bind to the cognate targeter unless the aptamer is bound by the appropriate drug; (ii) a targeter with an aptamer may not be able to bind to the cognate activator unless the aptamer is bound by the appropriate drug; and (iii) a targeter and an activator, each comprising a different aptamer that binds a different drug, may not be able to bind to each other unless both drugs are present. As illustrated by these examples, a dual guide nucleic acid (e.g., guide RNA) can be designed to be inducible.

Examples of aptamers and riboswitches can be found, for example, in: Nakamura et al., Genes Cells. 2012 May; 17(5):344-64; Vavalle et al., Future Cardiol. 2012 May; 8(3):371-82; Citartan et al., Biosens Bioelectron. 2012 Apr. 15; 34(1):1-11; and Liberman et al., Wiley Interdiscip Rev RNA. 2012 May-June; 3(3):369-84; all of which are herein incorporated by reference in their entirety.

Non-limiting examples of nucleotide sequences that can be included in a dual guide nucleic acid (e.g., guide RNA) are those disclosed in International Patent Application No. PCT/US2016/052690, or complements thereof that can hybridize to form a protein binding segment.

The guide nucleic acid can be single guide nucleic acid (e.g., single guide RNA) comprises two stretches of nucleotides (much like a “targeter” and an “activator” of a dual guide nucleic acid) that are complementary to one another, and hybridize to form the double stranded RNA duplex (dsRNA duplex) of the protein-binding segment (thus resulting in a stem-loop structure), and are covalently linked by intervening nucleotides (“linkers” or “linker nucleotides”). Thus, a single guide nucleic acid (e.g., a single guide RNA) can comprise a targeter and an activator, each having a duplex-forming segment, where the duplex-forming segments of the targeter and the activator hybridize with one another to form a dsRNA duplex. The targeter and the activator can be covalently linked via the 3′ end of the targeter and the 5′ end of the activator. Alternatively, targeter and the activator can be covalently linked via the 5′ end of the targeter and the 3′ end of the activator.

The linker of a single guide nucleic acid can have a length of from 3 nucleotides to 100 nucleotides. In some embodiments, the linker of a single guide nucleic acid (e.g., guide RNA) is about 3-10 nt, such as about 3-5 nucleotides (e.g., about 4 nt). Linker sequences are known in the art.

In some embodiments, one of the two complementary stretches of nucleotides of the single guide nucleic acid that form the dsRNA duplex is 60%, 65%, 70%, 75%, 80%, 85%, 90%, 95%, 98%, 99% or more identical or 100% identical to one of the activator (tracrRNA) molecules set forth in International Patent Application No. PCT/US2016/052690, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

In some embodiments, one of the two complementary stretches of nucleotides of the single guide nucleic acid (e.g., guide RNA) (or the DNA encoding the stretch) is 60%, 65%, 70%, 75%, 80%, 85%, 90%, 95%, 98%, 99% or more identical or 100% identical to one of the targeter (crRNA) sequences set forth in International Patent Application No. PCT/US2016/052690, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

In some embodiments, one of the two complementary stretches of nucleotides of the single guide nucleic acid (e.g., guide RNA) (or the DNA encoding the stretch) is 60%, 65%, 70%, 75%, 80%, 85%, 90%, 95%, 98%, 99% or more identical or 100% identical to one of the targeter (crRNA) sequences or activator (tracrRNA) sequences set forth in International Patent Application No. PCT/US2016/052690, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

Appropriate cognate pairs of targeters and activators can be routinely determined by taking into account the species name and base-pairing (for the dsRNA duplex of the protein-binding domain). Any activator/targeter pair can be used as part of dual guide nucleic acid (e.g., guide RNA) or as part of a single guide nucleic acid (e.g., guide RNA).

In some embodiments, an activator (e.g., a trRNA, trRNA-like molecule, etc.) of a dual guide nucleic acid (e.g., guide RNA) (e.g., a dual guide RNA) or a single guide nucleic acid (e.g., guide RNA) (e.g., a single guide RNA) includes a stretch of nucleotides with 60%, 65%, 70%, 75%, 80%, 85%, 90%, 95%, 98%, 99% or more, or 100% sequence identity with an activator (tracrRNA) molecule set forth in International Patent Application No. PCT/US2016/052690, or a complement thereof.

In some embodiments, an activator (e.g., a trRNA, trRNA-like molecule, etc.) of a dual guide nucleic acid (e.g., a dual guide RNA) or a single guide nucleic acid (e.g., a single guide RNA) includes 30 or more nucleotides (nt) (e.g., 40 or more, 50 or more, 60 or more, 70 or more, 75 or more nt). In some embodiments, an activator (e.g., a trRNA, trRNA-like molecule, etc.) of a dual guide nucleic acid (e.g., a dual guide RNA) or a single guide nucleic acid (e.g., a single guide RNA) has a length in a range of from 30 to 200 nucleotides (nt).

The protein-binding segment can have a length of from 10 nucleotides to 100 nucleotides.

Also with regard to both a subject single guide nucleic acid (e.g., single guide RNA) and to a subject dual guide nucleic acid (e.g., dual guide RNA), the dsRNA duplex of the protein-binding segment can have a length from 6 base pairs (bp) to 50 bp. The percent complementarity between the nucleotide sequences that hybridize to form the dsRNA duplex of the protein-binding segment can be 60% or more. For example, the percent complementarity between the nucleotide sequences that hybridize to form the dsRNA duplex of the protein-binding segment can be 65% or more, 70% or more, 75% or more, 80% or more, 85% or more, 90% or more, 95% or more, 98% or more, or 99% or more (e.g., in some embodiments, there are some nucleotides that do not hybridize and therefore create a bulge within the dsRNA duplex. In some embodiments, the percent complementarity between the nucleotide sequences that hybridize to form the dsRNA duplex of the protein-binding segment is 100%.

Hybrid Guide Nucleic Acids

In some embodiments, a guide nucleic acid is two RNA molecules (dual guide RNA). In some embodiments, a guide nucleic acid is one RNA molecule (single guide RNA). In some embodiments, a guide nucleic acid is a DNA/RNA hybrid molecule. In such embodiments, the protein-binding segment of the guide nucleic acid is RNA and forms an RNA duplex. Thus, the duplex-forming segments of the activator and the targeter is RNA. However, the targeting segment of a guide nucleic acid can be DNA. Thus, if a DNA/RNA hybrid guide nucleic acid is a dual guide nucleic acid, the “targeter” molecule and be a hybrid molecule (e.g., the targeting segment can be DNA and the duplex-forming segment can be RNA). In such embodiments, the duplex-forming segment of the “activator” molecule can be RNA (e.g., in order to form an RNA-duplex with the duplex-forming segment of the targeter molecule), while nucleotides of the “activator” molecule that are outside of the duplex-forming segment can be DNA (in which case the activator molecule is a hybrid DNA/RNA molecule) or can be RNA (in which case the activator molecule is RNA). If a DNA/RNA hybrid guide nucleic acid is a single guide nucleic acid, then the targeting segment can be DNA, the duplex-forming segments (which make up the protein-binding segment of the single guide nucleic acid) can be RNA, and nucleotides outside of the targeting and duplex-forming segments can be RNA or DNA.

A DNA/RNA hybrid guide nucleic can be useful in some embodiments, for example, when a target nucleic acid is an RNA. Cas9 normally associates with a guide RNA that hybridizes with a target DNA, thus forming a DNA-RNA duplex at the target site. Therefore, when the target nucleic acid is an RNA, it is sometimes advantageous to recapitulate a DNA-RNA duplex at the target site by using a targeting segment (of the guide nucleic acid) that is DNA instead of RNA. However, because the protein-binding segment of a guide nucleic acid is an RNA-duplex, the targeter molecule is DNA in the targeting segment and RNA in the duplex-forming segment. Hybrid guide nucleic acids can bias Cas9 binding to single stranded target nucleic acids relative to double stranded target nucleic acids.

Exemplary Guide Nucleic Acids

Exemplary Cas9 guide nucleic acids useful in the invention include any guide nucleic acid with a protein binding domain (e.g., tracrRNA) that binds to any Cas9 ortholog or variant, as described herein with respect to the Crisper Systems, below. Many Cas9 orthologs are known in the art, including, for instance, Streptococcus pyrogenes, Francisella tularensis (e.g., subsp. novicida), Pasteurella multocida, Neisseria meningitidis, Campylobacter jejuni, Streptococcus thermophilus (e.g. Streptococcus thermophilus #1, or Streptococcus thermophilus LMD-9 CRISPR 3), Campylobacter lari (e.g., Campylobacter lari CF89-12), Mycoplasma gallisepticum (e.g., str. F), Nitratifractor salsuginis (e.g., str DSM 16511), Parvibaculum lavamentivorans, Roseburia intestinalis, Neisseria cinerea, Gluconacetobacter diazotrophicus, Azospirillum B510, Sphaerochaeta globus (e.g., str. Buddy), Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Treponema denticola, Legionella pneumophila (e.g., str. Paris), Sutterella wadsworthensis, Corynebacter diphtheriae, and Staphylococcus aureus, among others. Additional Cas9 orthologs can be identified using available techniques and tools. orthogonal Cas9 proteins can be selected by examining and identifying divergent repeat sequences. Tools like CRISPRfinder (Grissa et al., Nucleic Acids Res 35: W52-W57 (2007), and CRISPRdb (Grissa et al., BMC Bioinformatics 8: 172 (2007) enable identification of CRISPR arrays with their constituent spacer and repeat sequences.

Thus, the Cas9 guide nucleic acid can, accordingly, comprise a protein binding segment of any of the foregoing microorganisms, or a variant thereof that retains the ability to bind a Cas9 protein, including variant proteins, as described herein with respect to the Crispr Systems. More specific examples of Cas9 guide nucleic acids include any comprising a protein binding domain (e.g., tracrRNA) comprising any of SEQ ID NOs: 7-31, or a variant thereof that retains the function of binding a Cas9 polypeptide. Variants can comprise, for instance, at least 60%, 65%, 70%, 75%, 80%, 85%, 90%, or 95% sequence identity to SEQ ID NOs: 7-31 (e.g., SEQ ID NOs: 7-31 with 1, 2, 3, 4, 5, 6, 7, 8, 9, 10, 11, 12, 13, 14, or 15 nucleotide substitutions, additions, or deletions).

In some embodiments, a suitable guide nucleic acid includes two separate RNA polynucleotide molecules. In some embodiments, the first of the two separate RNA polynucleotide molecules (the activator) comprises a nucleotide sequence having 60% or more (e.g., 65% or more, 70% or more, 75% or more, 80% or more, 85% or more, 90% or more, 95% or more, 98% or more, 99% or more, or 100%) nucleotide sequence identity over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides) to any one of the nucleotide sequences set forth in International Patent Application No. PCT/US2016/052690, or a complement thereof. In some embodiments, the second of the two separate RNA polynucleotide molecules (the targeter) comprises a nucleotide sequence having 60% or more (e.g., 65% or more, 70% or more, 75% or more, 80% or more, 85% or more, 90% or more, 95% or more, 98% or more, 99% or more, or 100%) nucleotide sequence identity over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides) to any one of the nucleotide sequences set forth in International Patent Application No. PCT/US2016/052690, or a complement thereof.

In some embodiments, a suitable guide nucleic acid is a single RNA polynucleotide and comprises first and second nucleotide sequence having 60% or more (e.g., 65% or more, 70% or more, 75% or more, 80% or more, 85% or more, 90% or more, 95% or more, 98% or more, 99% or more, or 100%) nucleotide sequence identity over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides) to any one of the nucleotide sequences set forth in International Patent Application No. PCT/US2016/052690, or complements thereof.

Yet another example of a guide RNA is a Cpf1 guide RNA (also known as a Cpf1 crRNA), which includes a target nucleic acid-binding segment and protein-binding segment including a duplex-forming segment in a single nucleic acid molecule. Cpf1 guide RNA can have a total length of from about 30 nucleotides (nt) to 100 nt, e.g., from 30 nt to 40 nt, from 40 nt to 45 nt, from 45 nt to 50 nt, from 50 nt to 60 nt, from 60 nt to 70 nt, from 70 nt to 80 nt, from 80 nt to 90 nt, or from 90 nt to 100 nt. In some embodiments, a Cpf1 guide RNA has a total length of 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, or 50 nt.

The target nucleic acid-binding segment of a Cpf1 guide RNA typically has a length of from 15 nt to 30 nt, e.g., 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt. In some embodiments, the target nucleic acid-binding segment has a length of 23 nt, 24 nt, or 25 nt.

The target nucleic acid-binding segment of a Cpf1 guide RNA can have 100% complementarity with a corresponding length of target nucleic acid sequence, or less than 100% complementarity with a corresponding length of target nucleic acid sequence provided the target binding segment hybridizes with the target nucleic acid (e.g., at least about 60%, 70%, 80%, 90%, 95%, or 99% sequence identity to the target nucleic acid sequence). By way of further illustration, the target nucleic acid binding segment of a Cpf1 guide RNA can have 1, 2, 3, 4, or 5 nucleotides that are not complementary to the target nucleic acid sequence, provided the sequences still will hybridize.

Exemplary Cpf1 guide nucleic acids include any having a protein binding domain that binds to any Cpf1 protein as described herein with respect to Crispr Systems, below. Cpf 1 orthologs from many different species are known, including, for instance, Lachnospiraceae bacterium (e.g., ND2006), Candidatus Methanomethylophilus alvus (e.g., Mx1201), Sneatia amnii (SaCpf1), Acidaminococcus (e.g., sp. BV3L6), Parcubacteria group bacterium (e.g., GW2011); Candidatus Roizmanbacteria bacterium (e.g., GW2011), Candidatus Peregrinbacterium bacterium (e.g., GW2011), Lachnospiracea bacterium (e.g., MA2020), Btyrivibrio (e.g. sp. NC3005), Butyrivibrio fibrisolvens, Prevotella bryantii (e.g., B14), Bacteroidetes oral taxon (e.g., 274), Flavobacterium brachiophilum (e.g., FL-15), Lachnospiraceae bacterium (e.g. MC2017), Moraxella lacunata, Moraxella bovoculi (e.g., AAX08_00205), Moraxella bovoculi (e.g., AAX11_00205), Francisella novicida (e.g., U112), and Thiomicrospira (e.g., sp. XS5). Additional Cpf1 orthologs can be identified using available techniques and tools. orthogonal Cpf1 proteins can be selected by examining and identifying divergent repeat sequences. Tools like CRISPRfinder (Grissa et al., Nucleic Acids Res 35: W52-W57 (2007), and CRISPRdb (Grissa et al., BMC Bioinformatics 8: 172 (2007) enable identification of CRISPR arrays with their constituent spacer and repeat sequences.

Thus, the Cpf1 guide nucleic acid can, accordingly, comprise a protein binding segment of any of the foregoing microorganisms, or a variant thereof that retains the ability to bind a Cpf1 protein, including variant proteins, as described herein with respect to the Crispr Systems.

In some embodiments, the duplex-forming segment of a Cpf1 guide RNA can have a length of from 15 nt to 25 nt, e.g., 15 nt, 16 nt, 17 nt, 18 nt, 19 nt, 20 nt, 21 nt, 22 nt, 23 nt, 24 nt, or 25 nt. In some embodiments, the duplex-forming segment of a Cpf1 guide RNA can comprise the nucleotide sequence 5′-AAUUUCUACUX₁X₂X₃UGUAGAU-3′ (SEQ ID NO: 32), wherein X₁, X₂, X₃ are each, independently, any amino acid:

X₁ can be absent or C, A, or G;
X₂ can be absent or G, A, or U; and

### X₃ can be G or U.

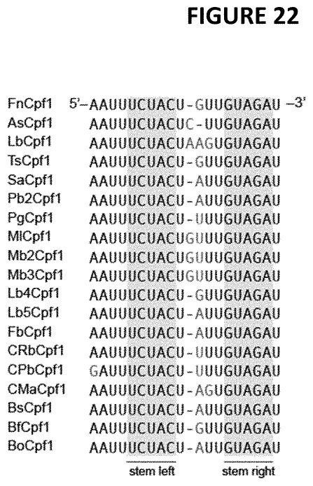

Specific examples of Cpf1 guide RNAs include those comprising a protein-binding segment comprising any of SEQ ID NOs: 33-51 (shown in FIG. 22), or a variant thereof that retains the function of binding a Cpf1 polypeptide. Variants can comprise, for instance, at least 60%, 65%, 70%, 75%, 80%, 85%, 90%, or 95% sequence identity to SEQ ID NOs: 33-51 (e.g., SEQ ID NOs: 33-51 with 1, 2, 3, 4, 5, 6, 7, or 8 nucleotide substitutions, additions, or deletions). In some embodiments, the Cpf1 guide RNA comprises at least the stem-sequences of SEQ ID NOs: 33-51 (see FIG. 22).

The Cpf1 guide RNA also will comprise a targeting segment the sequence of which is determined by the target nucleic acid to be edited.

### Linking and Extension

As demonstrated herein, the donor polynucleotide and the guide RNA can be advantageously linked together, either covalently or non-covalently. In some embodiments exemplified herein, the guide RNA and donor polynucleotide are covalently linked by, e.g., enzymatic or chemical ligation, or photoligation. In alternative embodiments also exemplified herein, the guide RNA and donor polynucleotide are non-covalently linked by, e.g., hybridization with each other, or with a bridge sequence.

Linkages can be facilitated, for example, through cycloaddition reactions (with or without a catalyst) between compatible functional groups. For instance, an azide or tetrazine functional group on one molecule can react with an alkyne, strained alkyne, or strained alkene on another molecule to form a linkage comprising a triazole or cyclic alkene group. Strained alkynes and strained alkenes include, for instance, any cycloalkyne or cycloalkene with sufficient strain to drive the cycloaddition reaction. Examples include groups comprising cyclooctynyl or cyclononynyl moieties, or cyclooctenyl or cyclononenyl moieities. Any of several functional groups known in the art can be used. In one embodiment, the strained alkyne or strained alkene is a dibeznocyclooctyne (DBCO), cyclooctene (e.g., trans-cyclooctene (TCO)), difluroocyclooctyne (DIFO), or dibenzocyclooctynol (DIBO) group:

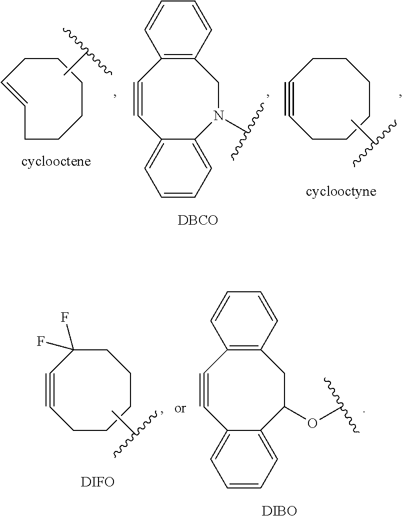

Similarly, non-limiting examples of linkages comprising a triazole or cyclic alkene moiety include the following:

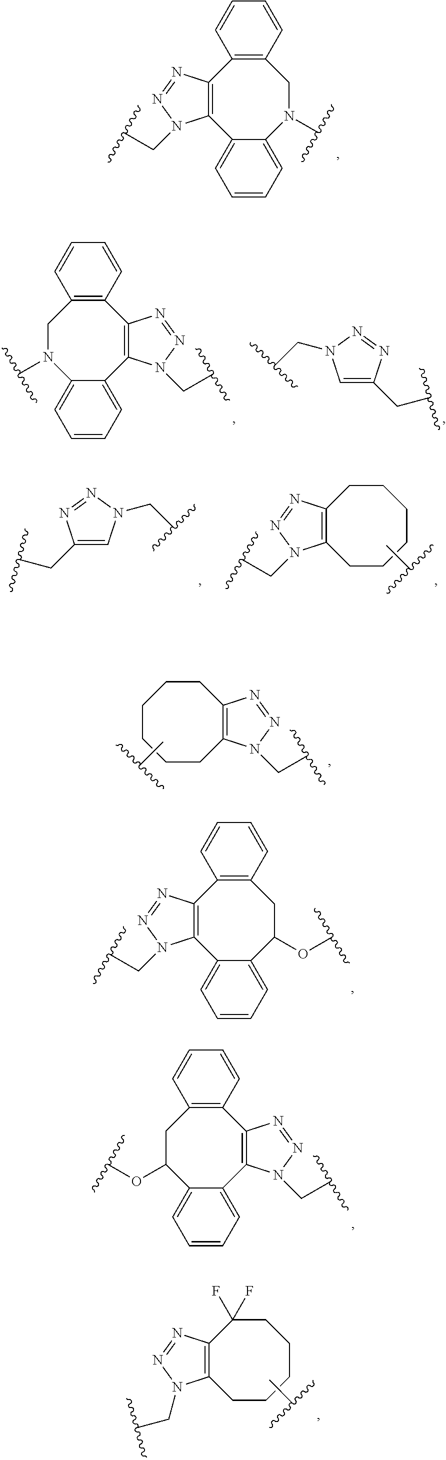

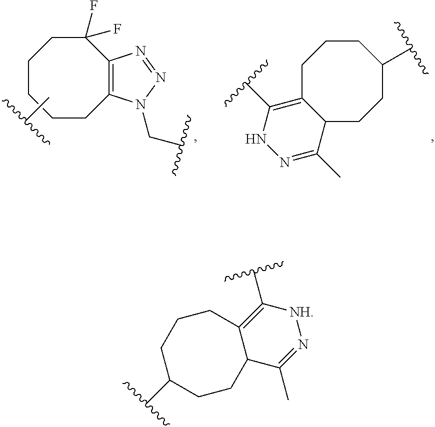

As further exemplified herein, both the 3′ and 5′ ends of the guide RNA are tolerant of a variety of modifications (e.g. amine, azide, thiol, alkyne, strained alkyne such as DBCO, strained alkene, tetrazine, and DNA conjugation) without consequent loss of activity. Accordingly, also contemplated herein are CRISPR systems comprising such modified guide RNAs. Remarkably, the 3′ and 5′ ends of the donor polynucleotide are also shown to be surprisingly tolerant of a number of modifications. Accordingly, also contemplated herein are CRISPR systems comprising such modified donor polynucleotides. As such, multiple ways of linking the guide RNA to the donor polynucleotide are contemplated and enabled by the present invention.

In some embodiments, the present disclosure contemplates a construct in which the donor nucleic acid is ligated to the guide nucleic acid. For instance, enzymatic ligases can be used to ligate the donor nucleic acid to the guide nucleic acid. Compatible temperature sensitive enzymatic ligases, include, but are not limited to, bacteriophage T4 ligase and E. coli ligase. Thermostable ligases include, but are not limited to, Afu ligase, Taq ligase, Tfl ligase, Tth ligase, Tth HB8 ligase, Thermus species AK16D ligase and Pfu ligase (see for example Published P.C.T. Application WO/2000/026381, Wu et al., Gene, 76(2):245-254, (1989), and Luo et al., Nucleic Acids Research, 24(15): 3071-3078 (1996)). The skilled artisan will appreciate that any number of thermostable ligases can be obtained from thermophilic or hyperthermophilic organisms, for example, certain species of eubacteria and archaea; and that such ligases can be employed in the disclosed methods and kits. In some embodiments, reversibly inactivated enzymes (see for example U.S. Pat. No. 5,773,258) can be employed in some embodiments of the present teachings.

In other embodiments, the present disclosure contemplates the use of chemical ligation agents. Chemical ligation agents include, without limitation, activating, condensing, and reducing agents, such as carbodiimide, cyanogen bromide (BrCN), N-hydroxysuccinimide esters, N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of the teachings herein. Detailed protocols for chemical ligation methods and descriptions of appropriate reactive groups can be found in, among other places, Xu et al., Nucleic Acid Res., 27:875-81 (1999); Gryaznov and Letsinger, Nucleic Acid Res. 21:1403-08 (1993); Gryaznov et al., Nucleic Acid Res. 22:2366-69 (1994); Kanaya and Yanagawa, Biochemistry 25:7423-30 (1986); Luebke and Dervan, Nucleic Acids Res. 20:3005-09 (1992); Sievers and von Kiedrowski, Nature 369:221-24 (1994); Liu and Taylor, Nucleic Acids Res. 26:3300-04 (1999); Wang and Kool, Nucleic Acids Res. 22:2326-33 (1994); Purmal et al., Nucleic Acids Res. 20:3713-19 (1992); Ashley and Kushlan, Biochemistry 30:2927-33 (1991); Chu and Orgel, Nucleic Acids Res. 16:3671-91 (1988); Sokolova et al., FEBS Letters 232:153-55 (1988); Naylor and Gilham, Biochemistry 5:2722-28 (1966); and U.S. Pat. No. 5,476,930.

In some embodiments, the methods, kits and compositions of the present disclosure are also compatible with photoligation reactions. Photoligation using light of an appropriate wavelength as a ligation agent is also within the scope of the teachings. In some embodiments, photoligation comprises probes comprising nucleotide analogs, including but not limited to, 4-thiothymidine, 5-vinyluracil and its derivatives, or combinations thereof. In some embodiments, the ligation agent comprises: (a) light in the UV-A range (about 320 nm to about 400 nm), the UV-B range (about 290 nm to about 320 nm), or combinations thereof, (b) light with a wavelength between about 300 nm and about 375 nm, (c) light with a wavelength of about 360 nm to about 370 nm; (d) light with a wavelength of about 364 nm to about 368 nm, or (e) light with a wavelength of about 366 nm. In some embodiments, photoligation is reversible. Descriptions of photoligation can be found in, among other places, Fujimoto et al., Nucl. Acid Symp. Ser. 42:39-40 (1999); Fujimoto et al., Nucl. Acid Res. Suppl. 1:185-86 (2001); Fujimoto et al., Nucl. Acid Suppl., 2:155-56 (2002); Liu and Taylor, Nucl. Acid Res. 26:3300-04 (1998) and on the world wide web at: sbchem.kyoto-u.ac.jp/saito-lab.

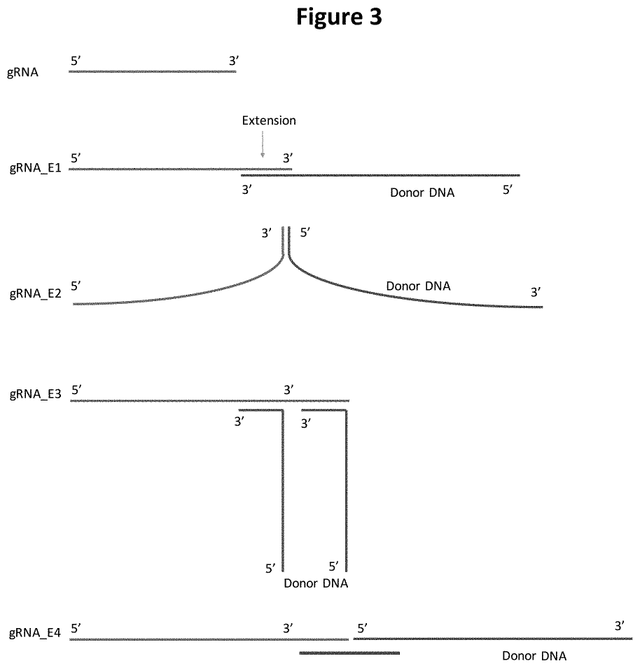

In another embodiment, the guide nucleic acid is hybridized to the donor nucleic acid. For instance, the guide nucleic acid (e.g., guide RNA) can comprise a segment with a nucleotide sequence that is sufficiently complementary to a segment of the donor nucleic acid to facilitate hybridization. For instance, the guide RNA can comprise a segment of from 10 to 50 nucleotides (e.g., from 10 nucleotides (nt) to 15 nt, from 15 nt to 20 nt, from 20 nt to 25 nt, from 25 nt to 30 nt, from 30 nt to 40 nt, or from 40 nt to 50 nt) with at least 50%, at least 60%, at least 70%, at least 80%, at least 90%, at least 95%, at least 98%, at least 99%, or 100% sequence identity to a region of the donor polynucleotide sequence, such that they hybridize directly together. This segment can be added to the guide RNA as an extension to the guide RNA sequence. The hybridizing segments can be present at any suitable position of the molecule, such at the 5′ or 3′ end of the guide nucleic acid, and the 5′ or 3′ end of the donor nucleic acid. The guide nucleic acid further can comprise multiple hybridization segments to allow hybridization of multiple donor nucleic acids to a single guide nucleic acid. Any number of alternative hybridization configurations are possible, including those illustrated in FIG. 3.

Alternatively, the guide nucleic acid and donor polynucleotide may each hybridize to a bridge sequence, also as demonstrated herein. The bridge sequence can comprise, for instance, a first segment that is sufficiently complementary to a segment of the guide nucleic acid to facilitate hybridization, and a second segment that is sufficiently complementary to a segment of the guide nucleic acid to facilitate hybridization, optionally with a non-hybridizing region therebetween. In some embodiments, the first and second segments of the bridge sequence, and optional non-hybridizing region therebetween, each are 10 to 50 nucleotides (e.g., from 10 nucleotides (nt) to 15 nt, from 15 nt to 20 nt, from 20 nt to 25 nt, from 25 nt to 30 nt, from 30 nt to 40 nt, or from 40 nt to 50 nt). Further, each of the hybridizing segments of the bridge sequence has at least 50%, at least 60%, at least 70%, at least 80%, at least 90%, at least 95%, at least 98%, at least 99%, or 100%, nucleotide sequence identity to a the guide RNA and the donor polynucleotide, respectively.

Extensions to the guide nucleic acid are believed to improve delivery of the nucleic acid by increasing the molecular weight or negative charge of the gRNA. Furthermore, the addition of bases to the 3′ end can increase the half-life of functionally important gRNA sequence. The guide nucleic acid provided herein can comprise a nucleotide extension that does not necessarily hybridize to a donor polynucleotide, instead or in addition to an extension sequence that hybridizes the donor sequence. For instance, the guide nucleic acid can comprise a 3′ or 5′ nucleotide extension (e.g., a nucleotide extension on the 3′ end, 5′ end or both of a Cpf1 guide nucleic acid, or a nucleotide extension on the 3′ end, 5′ end or both of a Cas9 guide nucleic acid) of about 20 nucleotides or more, 30 nucleotides or more, 40 nucleotides or more, 50 nucleotides or more, 60 nucleotides or more, 70 nucleotides or more, 80 nucleotides or more, or even 100 nucleotides or more. Typically, the nucleotide extension will be less than about 1000 nucleotides, and, in some cases, less than about 500 nucleotides (e.g., less than about 250 nucleotides.

### Crispr Systems

There are at least five main CRISPR system types (Type I, II, III, IV and V) and at least 16 distinct subtypes (Makarova, K. S., et al., Nat Rev Microbiol. 2015. Nat. Rev. Microbiol. 13, 722-736). CRISPR systems are also classified based on their effector proteins. Class 1 systems possess multi-subunit crRNA-effector complexes, whereas in class 2 systems all functions of the effector complex are carried out by a single protein (e.g., Cas9 or Cpf1). In some embodiments, the present disclosure teaches using type II and/or type V single-subunit effector systems. Thus, in some embodiments, the present disclosure teaches using class 2 CRISPR systems.

### Type II CRISPR Systems

In some embodiments, the present disclosure provides compositions and method using a Type II CRISPR system, e.g., a Cas9 polypeptide or an nucleic acid (e.g., mRNA) encoding the same. In some embodiments, the present disclosure teaches Cas9 Type II CRISPR systems. Type II systems rely on a i) single endonuclease protein, ii) a transactiving crRNA (tracrRNA), and iii) a crRNA where a 20-nucleotide (nt) portion of the 5′ end of crRNA is complementary to a target nucleic acid. Cas9 endonucleases produce blunt end DNA breaks, and are recruited to target DNA by a combination of a crRNA and a tracrRNA oligos, which tether the endonuclease via complementary hybridization of the RNA complex.

In some embodiments, DNA recognition by the crRNA/endonuclease complex requires additional complementary base-pairing with a protospacer adjacent motif (PAM) (e.g., 5′-NGG-3′) located in a 3′ portion of the target DNA, downstream from the target protospacer. (Jinek, M., Et. al., Science. 2012:337; 816-821). The particular PAM motif recognized by a crRNA/endonuclease complex is different for different RNA-guided endonuclease proteins.

Any Cas9 polypeptide can be used. Suitable Cas9 polypeptides for inclusion in a complex of the present disclosure include a naturally-occurring Cas9 polypeptide (e.g., naturally occurs in bacterial and/or archaeal cells), or a non-naturally-occurring Cas9 polypeptide (e.g., the Cas9 polypeptide is a variant Cas9 polypeptide, a chimeric polypeptide as discussed below, and the like), as described below. One skilled in the art can appreciate that the Cas9 polypeptide can be any variant derived or isolated from any source. Many Cas9 orthologs are known in the art, including, for instance, Streptococcus pyrogenes, Francisella tularensis (e.g., subsp. novicida), Pasteurella multocida, Neisseria meningitidis, Campylobacter jejuni, Streptococcus thermophilus (e.g. Streptococcus thermophilus #1, or Streptococcus thermophilus LMD-9 CRISPR 3), Campylobacter lari (e.g., Campylobacter lari CF89-12), Mycoplasma gallisepticum (e.g., str. F), Nitratifractor salsuginis (e.g., str DSM 16511), Parvibaculum lavamentivorans, Roseburia intestinalis, Neisseria cinerea, Gluconacetobacter diazotrophicus, Azospirillum B510, Sphaerochaeta globus (e.g., str. Buddy), Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Treponema denticola, Legionella pneumophila (e.g., str. Paris), Sutterella wadsworthensis, Corynebacter diphtheriae, and Staphylococcus aureus, among others. Additional Cas9 orthologs can be identified using available techniques and tools. orthogonal Cas9 proteins can be selected by examining and identifying divergent repeat sequences. Tools like CRISPRfinder (Grissa et al., Nucleic Acids Res 35: W52-W57 (2007), and CRISPRdb (Grissa et al., BMC Bioinformatics 8: 172 (2007) enable identification of CRISPR arrays with their constituent spacer and repeat sequences.

The Cas9 protein also can be any variant of a naturally occurring Cas9 protein. For example, the Cas9 peptide of the present disclosure can include one or more of the mutations described in the literature, including but not limited to the functional mutations described in: Fonfara et al. Nucleic Acids Res. 2014 February; 42(4):2577-90; Nishimasu H. et al. Cell. 2014 Feb. 27; 156(5):935-49; Jinek M. et al. Science. 2012 337:816-21; and Jinek M. et al. Science. 2014 Mar. 14; 343(6176); Makarova et al., Cell, 168, DOI http://dx.doi.org.10.1016/j.ce11.2016.12.038 (Jan. 12, 2017); see also U.S. patent application Ser. No. 13/842,859, filed Mar. 15, 2013, which is hereby incorporated by reference; further, see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference. In some embodiments, the systems and methods disclosed herein can be used with the wild type Cas9 protein having double-stranded nuclease activity. In other embodiments, a Cas9 mutant that act as a single stranded nickase, or other mutant with modified nuclease activity, is used. As such, a Cas9 polypeptide that is suitable for inclusion in a complex (e.g., an encapsulated complex) of the present disclosure can be an enzymatically active Cas9 polypeptide, e.g., can make single- or double-stranded breaks in a target nucleic acid, or alternatively can have reduced enzymatic activity compared to a wild-type Cas9 polypeptide.

Naturally occurring Cas9 polypeptides bind a guide nucleic acid, are thereby directed to a specific sequence within a target nucleic acid (a target site), and cleave the target nucleic acid (e.g., cleave dsDNA to generate a double strand break, cleave ssDNA, cleave ssRNA, etc.). A subject Cas9 polypeptide comprises two portions, an RNA-binding portion and an activity portion. The RNA-binding portion interacts with a subject guide nucleic acid, and an activity portion exhibits site-directed enzymatic activity (e.g., nuclease activity, activity for DNA and/or RNA methylation, activity for DNA and/or RNA cleavage, activity for histone acetylation, activity for histone methylation, activity for RNA modification, activity for RNA-binding, activity for RNA splicing etc. In some embodiments the activity portion exhibits reduced nuclease activity relative to the corresponding portion of a wild type Cas9 polypeptide. In some embodiments, the activity portion is enzymatically inactive.

Assays to determine whether a protein has an RNA-binding portion that interacts with a subject guide nucleic acid can be any convenient binding assay that tests for binding between a protein and a nucleic acid. Exemplary binding assays include binding assays (e.g., gel shift assays) that involve adding a guide nucleic acid and a Cas9 polypeptide to a target nucleic acid.

Assays to determine whether a protein has an activity portion (e.g., to determine if the polypeptide has nuclease activity that cleave a target nucleic acid) can be any convenient nucleic acid cleavage assay that tests for nucleic acid cleavage. Exemplary cleavage assays that include adding a guide nucleic acid and a Cas9 polypeptide to a target nucleic acid.

In some embodiments, a suitable Cas9 polypeptide for inclusion in a complex of the present disclosure has enzymatic activity that modifies target nucleic acid (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity).

In other embodiments, a suitable Cas9 polypeptide for inclusion in a complex of the present disclosure has enzymatic activity that modifies a polypeptide (e.g., a histone) associated with target nucleic acid (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

Many Cas9 orthologues from a wide variety of species have been identified, as discussed above. In some instances, the orthologous proteins share only a few identical amino acids. Yet, most identified Cas9 orthologues have the same domain architecture with a central HNH endonuclease domain and a split RuvC/RNaseH domain. Cas9 proteins typically share 4 key motifs with a conserved architecture. Motifs 1, 2, and 4 are RuvC like motifs while motif 3 is an HNH-motif.

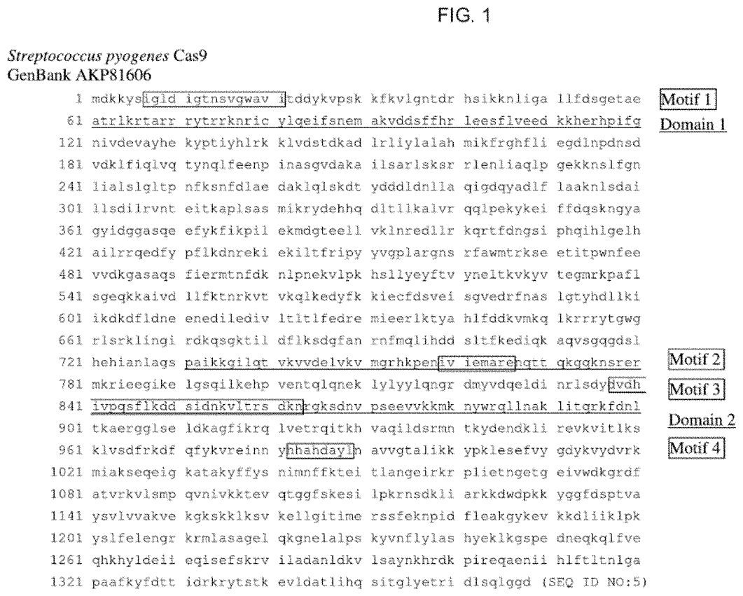

In some embodiments, a suitable Cas9 polypeptide comprises an amino acid sequence having 4 motifs (motifs 1-4), wherein each of motifs 1-4 having 60% or more, 70% or more, 75% or more, 80% or more, 85% or more, 90% or more, 95% or more, 99% or more or 100% amino acid sequence identity to the corresponding motif of the Cas9 amino acid sequence depicted in FIG. 1 (SEQ ID NO:1); or, alternatively, to motifs 1-4 of the Cas9 amino acid sequence depicted in Table 1 below (motifs 1-4 of SEQ ID NO:1 are SEQ ID NOs: 3-6, respectively, as depicted in Table 1 below); or alternatively to amino acids 7-166 or 731-1003 of the Cas9 amino acid sequence depicted in FIG. 1 (SEQ ID NO:1)

In some embodiments, a Cas9 polypeptide comprises an amino acid sequence having 75% or more, 80% or more, 85% or more, 90% or more, 95% or more, or 98%, amino acid sequence identity to the amino acid sequence depicted in FIG. 1 and set forth in SEQ ID NO:1; and comprises amino acid substitutions of N497, R661, Q695, and Q926 relative to the amino acid sequence set forth in SEQ ID NO:1; or comprises an amino acid substitution of K855 relative to the amino acid sequence set forth in SEQ ID NO:1; or comprises amino acid substitutions of K810, K1003, and R1060 relative to the amino acid sequence set forth in SEQ ID NO:1; or comprises amino acid substitutions of K848, K1003, and R1060 relative to the amino acid sequence set forth in SEQ ID NO:1.

As used herein, the term “Cas9 polypeptide” encompasses the term “variant Cas9 polypeptide”; and the term “variant Cas9 polypeptide” encompasses the term “chimeric Cas9 polypeptide.”

### Variant Cas9 Polypeptides

A suitable Cas9 polypeptides for inclusion in a complex of the present disclosure includes a variant Cas9 polypeptide. A variant Cas9 polypeptide has an amino acid sequence that is different by one amino acid (e.g., has a deletion, insertion, substitution, fusion) (i.e., different by at least one amino acid) when compared to the amino acid sequence of a wild type Cas9 polypeptide (e.g., a naturally occurring Cas9 polypeptide, as described above). In some instances, the variant Cas9 polypeptide has an amino acid change (e.g., deletion, insertion, or substitution) that reduces the nuclease activity of the Cas9 polypeptide. For example, in some instances, the variant Cas9 polypeptide has less than 50%, less than 40%, less than 30%, less than 20%, less than 10%, less than 5%, or less than 1% of the nuclease activity of the corresponding wild-type Cas9 polypeptide. In some embodiments, the variant Cas9 polypeptide has no substantial nuclease activity. When a Cas9 polypeptide is a variant Cas9 polypeptide that has no substantial nuclease activity, it can be referred to as “dCas9.”

In some embodiments, a variant Cas9 polypeptide has reduced nuclease activity. For example, a variant Cas9 polypeptide suitable for use in a binding method of the present disclosure exhibits less than about 20%, less than about 15%, less than about 10%, less than about 5%, less than about 1%, or less than about 0.1%, of the endonuclease activity of a wild-type Cas9 polypeptide, e.g., a wild-type Cas9 polypeptide comprising an amino acid sequence as depicted in FIG. 1 (SEQ ID NO:1).

In some embodiments, a variant Cas9 polypeptide can cleave the complementary strand of a target nucleic acid but has reduced ability to cleave the non-complementary strand of a double stranded target nucleic acid. For example, the variant Cas9 polypeptide can have a mutation (amino acid substitution) that reduces the function of the RuvC domain (e.g., “domain 1” of FIG. 1). As a non-limiting example, in some embodiments, a variant Cas9 polypeptide has a D10A mutation (e.g., aspartate to alanine at an amino acid position corresponding to position 10 of SEQ ID NO:1) and can therefore cleave the complementary strand of a double stranded target nucleic acid but has reduced ability to cleave the non-complementary strand of a double stranded target nucleic acid (thus resulting in a single strand break (SSB) instead of a double strand break (DSB) when the variant Cas9 polypeptide cleaves a double stranded target nucleic acid) (see, for example, Jinek et al., Science. 2012 Aug. 17; 337(6096):816-21).

In some embodiments, a variant Cas9 polypeptide can cleave the non-complementary strand of a double stranded target nucleic acid but has reduced ability to cleave the complementary strand of the target nucleic acid. For example, the variant Cas9 polypeptide can have a mutation (amino acid substitution) that reduces the function of the HNH domain (RuvC/HNH/RuvC domain motifs, “domain 2” of FIG. 1). As a non-limiting example, in some embodiments, the variant Cas9 polypeptide can have an H840A mutation (e.g., histidine to alanine at an amino acid position corresponding to position 840 of SEQ ID NO:1) (FIG. 1) and can therefore cleave the non-complementary strand of the target nucleic acid but has reduced ability to cleave the complementary strand of the target nucleic acid (thus resulting in a SSB instead of a DSB when the variant Cas9 polypeptide cleaves a double stranded target nucleic acid). Such a Cas9 polypeptide has a reduced ability to cleave a target nucleic acid (e.g., a single stranded target nucleic acid) but retains the ability to bind a target nucleic acid (e.g., a single-stranded or a double-stranded target nucleic acid).

In some embodiments, a variant Cas9 polypeptide has a reduced ability to cleave both the complementary and the non-complementary strands of a double stranded target nucleic acid. As a non-limiting example, in some embodiments, the variant Cas9 polypeptide harbors both the D10A and the H840A mutations (e.g., mutations in both the RuvC domain and the HNH domain) such that the polypeptide has a reduced ability to cleave both the complementary and the non-complementary strands of a double stranded target nucleic acid. Such a Cas9 polypeptide has a reduced ability to cleave a target nucleic acid (e.g., a single-stranded target nucleic acid or a double-stranded target nucleic acid) but retains the ability to bind a target nucleic acid (e.g., a single stranded target nucleic acid or a double-stranded target nucleic acid).

As another non-limiting example, in some embodiments, the variant Cas9 polypeptide harbors W476A and W1126A mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors P475A, W476A, N477A, D1125A, W1126A, and D1127A mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors H840A, W476A, and W1126A, mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors H840A, D10A, W476A, and W1126A, mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors, H840A, P475A, W476A, N477A, D1125A, W1126A, and D1127A mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors D10A, H840A, P475A, W476A, N477A, D1125A, W1126A, and D1127A mutations such that the polypeptide has a reduced ability to cleave a target nucleic acid. Such a Cas9 polypeptide has a reduced ability to cleave a target nucleic acid but retains the ability to bind a target nucleic acid.

Other residues can be mutated to achieve the above effects (i.e. inactivate one or the other nuclease portions). As non-limiting examples, residues D10, G12, G17, E762, H840, N854, N863, H982, H983, A984, D986, and/or A987 can be altered (i.e., substituted) (see Table 1 for more information regarding the conservation of Cas9 amino acid residues). Also, mutations other than alanine substitutions are suitable.

In some embodiments, a variant Cas9 polypeptide that has reduced catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the variant Cas9 polypeptide can still bind to target nucleic acid in a site-specific manner (because it is still guided to a target nucleic acid sequence by a guide nucleic acid) as long as it retains the ability to interact with the guide nucleic acid.

TABLE 1
Table 1 lists 4 motifs that are present in Cas9 sequences
from various species. The amino acids listed here are from
the Cas9 from S. pyogenes (SEQ ID NO: 1).

| | | | Highly |
|---|---|---|---|
| Motif | Motif | Amino acids (residue #s) | conserved |
| 1 | RuvC | IGLDIGTNSVGWAVI (7-21) | D10, G12, |
| | | (SEQ ID NO: 3) | G17 |
| 2 | RuvC | IVIEMARE (759-766) | E762 |
| | | (SEQ ID NO: 4) | |
| 3 | HNH- | DVDHIVPQSFLKDDSIDNKVLTRSDKN | H840, |
| | motif | (837-863) (SEQ ID NO: 5) | N854, N863 |
| 4 | RuvC | HHAHDAYL (982-989) | H982, H983, |
| | | (SEQ ID NO: 6) | A984, |
| | | | D986, A987 |

In addition to the above, a variant Cas9 protein can have the same parameters for sequence identity as described above for Cas9 polypeptides. Thus, in some embodiments, a suitable variant Cas9 polypeptide comprises an amino acid sequence having 4 motifs, each of motifs 1-4 having 60% or more, 70% or more, 75% or more, 80% or more, 85% or more, 90% or more, 95% or more, 99% or more or 100% amino acid sequence identity of the Cas9 amino acid sequence depicted in FIG. 1 (SEQ ID NO:1), or alternatively to motifs 1-4 (motifs 1-4 of SEQ ID NO:1 are SEQ ID NOs:3-6, respectively, as depicted in Table 1); or alternatively to amino acids 7-166 or 731-1003 of the Cas9 amino acid sequence depicted in FIG. 1 (SEQ ID NO:1. Any Cas9 protein as defined above can be used as a Cas9 polypeptide, or as part of a chimeric Cas9 polypeptide, in a complex of the present disclosure, including those specifically referenced in International Patent Application No. PCT/US2016/052690.

In some embodiments, a suitable variant Cas9 polypeptide comprises an amino acid sequence having 60% or more, 70% or more, 75% or more, 80% or more, 85% or more, 90% or more, 95% or more, 99% or more, or 100% amino acid sequence identity to the Cas9 amino acid sequence depicted in FIG. 1 (SEQ ID NO:1). Any Cas9 protein as defined above can be used as a variant Cas9 polypeptide or as part of a chimeric variant Cas9 polypeptide in a complex of the present disclosure, including those specifically referenced in International Patent Application No. PCT/US2016/052690.

### Chimeric Polypeptides (Fusion Polypeptides)

In some embodiments, a variant Cas9 polypeptide is a chimeric Cas9 polypeptide (also referred to herein as a fusion polypeptide, e.g., a “Cas9 fusion polypeptide”). A Cas9 fusion polypeptide can bind and/or modify a target nucleic acid (e.g., cleave, methylate, demethylate, etc.) and/or a polypeptide associated with target nucleic acid (e.g., methylation, acetylation, etc., of, for example, a histone tail).

A Cas9 fusion polypeptide is a variant Cas9 polypeptide by virtue of differing in sequence from a wild type Cas9 polypeptide (e.g., a naturally occurring Cas9 polypeptide). A Cas9 fusion polypeptide is a Cas9 polypeptide (e.g., a wild type Cas9 polypeptide, a variant Cas9 polypeptide, a variant Cas9 polypeptide with reduced nuclease activity (as described above), and the like) fused to a covalently linked heterologous polypeptide (also referred to as a “fusion partner”). In some embodiments, a Cas9 fusion polypeptide is a variant Cas9 polypeptide with reduced nuclease activity (e.g., dCas9) fused to a covalently linked heterologous polypeptide. In some embodiments, the heterologous polypeptide exhibits (and therefore provides for) an activity (e.g., an enzymatic activity) that will also be exhibited by the Cas9 fusion polypeptide (e.g., methyltransferase activity, acetyltransferase activity, kinase activity, ubiquitinating activity, etc.). In some such embodiments, a method of binding, e.g., where the Cas9 polypeptide is a variant Cas9 polypeptide having a fusion partner (i.e., having a heterologous polypeptide) with an activity (e.g., an enzymatic activity) that modifies the target nucleic acid, the method can also be considered to be a method of modifying the target nucleic acid. In some embodiments, a method of binding a target nucleic acid (e.g., a single stranded target nucleic acid) can result in modification of the target nucleic acid. Thus, in some embodiments, a method of binding a target nucleic acid (e.g., a single stranded target nucleic acid) can be a method of modifying the target nucleic acid.

In some embodiments, the heterologous sequence provides for subcellular localization, i.e., the heterologous sequence is a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an endoplasmic reticulum (ER) retention signal, and the like). In some embodiments, a variant Cas9 does not include a NLS so that the protein is not targeted to the nucleus (which can be advantageous, e.g., when the target nucleic acid is an RNA that is present in the cytosol). In some embodiments, the heterologous sequence can provide a tag (i.e., the heterologous sequence is a detectable label) for ease of tracking and/or purification (e.g., a fluorescent protein, e.g., green fluorescent protein (GFP), YFP, RFP, CFP, mCherry, tdTomato, and the like; a histidine tag, e.g., a 6×His tag; a hemagglutinin (HA) tag; a FLAG tag; a Myc tag; and the like). In some embodiments, the heterologous sequence can provide for increased or decreased stability (i.e., the heterologous sequence is a stability control peptide, e.g., a degron, which in some embodiments is controllable (e.g., a temperature sensitive or drug controllable degron sequence, see below). In some embodiments, the heterologous sequence can provide for increased or decreased transcription from the target nucleic acid (i.e., the heterologous sequence is a transcription modulation sequence, e.g., a transcription factor/activator or a fragment thereof, a protein or fragment thereof that recruits a transcription factor/activator, a transcription repressor or a fragment thereof, a protein or fragment thereof that recruits a transcription repressor, a small molecule/drug-responsive transcription regulator, etc.). In some embodiments, the heterologous sequence can provide a binding domain (i.e., the heterologous sequence is a protein binding sequence, e.g., to provide the ability of a Cas9 fusion polypeptide to bind to another protein of interest, e.g., a DNA or histone modifying protein, a transcription factor or transcription repressor, a recruiting protein, an RNA modifaction enzyme, an RNA-binding protein, a translation initation factor, an RNA splicing factor, etc.). A heterologous nucleic acid sequence may be linked to another nucleic acid sequence (e.g., by genetic engineering) to generate a chimeric nucleotide sequence encoding a chimeric polypeptide.

A subject Cas9 fusion polypeptide (Cas9 fusion protein) can have multiple (1 or more, 2 or more, 3 or more, etc.) fusion partners in any combination of the above. As an illustrative example, a Cas9 fusion protein can have a heterologous sequence that provides an activity (e.g., for transcription modulation, target modification, modification of a protein associated with a target nucleic acid, etc.) and can also have a subcellular localization sequence. In some embodiments, such a Cas9 fusion protein might also have a tag for ease of tracking and/or purification (e.g., green fluorescent protein (GFP), YFP, RFP, CFP, mCherry, tdTomato, and the like; a histidine tag, e.g., a 6×His tag; a hemagglutinin (HA) tag; a FLAG tag; a Myc tag; and the like). As another illustrative example, a Cas9 protein can have one or more NLSs (e.g., two or more, three or more, four or more, five or more, 1, 2, 3, 4, or 5 NLSs). In some embodiments a fusion partner (or multiple fusion partners) (e.g., an NLS, a tag, a fusion partner providing an activity, etc.) is located at or near the C-terminus of Cas9. In some embodiments a fusion partner (or multiple fusion partners) (e.g., an NLS, a tag, a fusion partner providing an activity, etc.) is located at the N-terminus of Cas9. In some embodiments a Cas9 has a fusion partner (or multiple fusion partners)(e.g., an NLS, a tag, a fusion partner providing an activity, etc.) at both the N-terminus and C-terminus.

Suitable fusion partners that provide for increased or decreased stability include, but are not limited to degron sequences. Degrons are readily understood by one of ordinary skill in the art to be amino acid sequences that control the stability of the protein of which they are part. For example, the stability of a protein comprising a degron sequence is controlled in part by the degron sequence. In some embodiments, a suitable degron is constitutive such that the degron exerts its influence on protein stability independent of experimental control (i.e., the degron is not drug inducible, temperature inducible, etc.) In some embodiments, the degron provides the variant Cas9 polypeptide with controllable stability such that the variant Cas9 polypeptide can be turned “on” (i.e., stable) or “off” (i.e., unstable, degraded) depending on the desired conditions. For example, if the degron is a temperature sensitive degron, the variant Cas9 polypeptide may be functional (i.e., “on”, stable) below a threshold temperature (e.g., 42° C., 41° C., 40° C., 39° C., 38° C., 37° C., 36° C., 35° C., 34° C., 33° C., 32° C., 31° C., 30° C., etc.) but non-functional (i.e., “off”, degraded) above the threshold temperature. As another example, if the degron is a drug inducible degron, the presence or absence of drug can switch the protein from an “off” (i.e., unstable) state to an “on” (i.e., stable) state or vice versa. An exemplary drug inducible degron is derived from the FKBP12 protein. The stability of the degron is controlled by the presence or absence of a small molecule that binds to the degron.

Examples of suitable degrons include, but are not limited to those degrons controlled by Shield-1, DHFR, auxins, and/or temperature. Non-limiting examples of suitable degrons are known in the art (e.g., Dohmen et al., Science, 1994. 263(5151): p. 1273-1276: Heat-inducible degron: a method for constructing temperature-sensitive mutants; Schoeber et al., Am J Physiol Renal Physiol. 2009 January; 296(1):F204-11: Conditional fast expression and function of multimeric TRPV5 channels using Shield-1 Chu et al., Bioorg Med Chem Lett. 2008 Nov. 15; 18(22):5941-4: Recent progress with FKBP-derived destabilizing domains Kanemaki, Pflugers Arch. 2012 Dec. 28: Frontiers of protein expression control with conditional degrons; Yang et al., Mol Cell. 2012 Nov. 30; 48(4):487-8: Titivated for destruction: the methyl degron; Barbour et al., Biosci Rep. 2013 Jan. 18; 33(1): Characterization of the bipartite degron that regulates ubiquitin-independent degradation of thymidylate synthase; and Greussing et al., J Vis Exp. 2012 Nov. 10; (69): Monitoring of ubiquitin-proteasome activity in living cells using a Degron (dgn)-destabilized green fluorescent protein (GFP)-based reporter protein; all of which are hereby incorporated in their entirety by reference).

Exemplary degron sequences have been well-characterized and tested in both cells and animals. Thus, fusing Cas9 (e.g., wild type Cas9; variant Cas9; variant Cas9 with reduced nuclease activity, e.g., dCas9; and the like) to a degron sequence produces a “tunable” and “inducible” Cas9 polypeptide. Any of the fusion partners described herein can be used in any desirable combination. As one non-limiting example to illustrate this point, a Cas9 fusion protein (i.e., a chimeric Cas9 polypeptide) can comprise a YFP sequence for detection, a degron sequence for stability, and transcription activator sequence to increase transcription of the target nucleic acid. A suitable reporter protein for use as a fusion partner for a Cas9 polypeptide (e.g., wild type Cas9, variant Cas9, variant Cas9 with reduced nuclease function, etc.), includes, but is not limited to, the following exemplary proteins (or functional fragment thereof): his3, β-galactosidase, a fluorescent protein (e.g., GFP, RFP, YFP, cherry, tomato, etc., and various derivatives thereof), luciferase, β-glucuronidase, and alkaline phosphatase. Furthermore, the number of fusion partners that can be used in a Cas9 fusion protein is unlimited. In some embodiments, a Cas9 fusion protein comprises one or more (e.g. two or more, three or more, four or more, or five or more) heterologous sequences.

Suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity, any of which can be directed at modifying nucleic acid directly (e.g., methylation of DNA or RNA) or at modifying a nucleic acid-associated polypeptide (e.g., a histone, a DNA binding protein, and RNA binding protein, and the like). Further suitable fusion partners include, but are not limited to boundary elements (e.g., CTCF), proteins and fragments thereof that provide periphery recruitment (e.g., Lamin A, Lamin B, etc.), and protein docking elements (e.g., FKBP/FRB, Pil1/Aby1, etc.).

Examples of various additional suitable fusion partners (or fragments thereof) for a subject variant Cas9 polypeptide include, but are not limited to those described in the PCT patent applications: WO2010/075303, WO2012/068627, and WO2013/155555 which are hereby incorporated by reference in their entirety.

Suitable fusion partners include, but are not limited to, a polypeptide that provides an activity that indirectly increases transcription by acting directly on the target nucleic acid or on a polypeptide (e.g., a histone, a DNA-binding protein, an RNA-binding protein, an RNA editing protein, etc.) associated with the target nucleic acid. Suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity.

Additional suitable fusion partners include, but are not limited to, a polypeptide that directly provides for increased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.).

Non-limiting examples of fusion partners to accomplish increased or decreased transcription include transcription activator and transcription repressor domains (e.g., the Krüppel associated box (KRAB or SKD); the Mad mSIN3 interaction domain (SID); the ERF repressor domain (ERD), etc.). In some such embodiments, a Cas9 fusion protein is targeted by the guide nucleic acid to a specific location (i.e., sequence) in the target nucleic acid and exerts locus-specific regulation such as blocking RNA polymerase binding to a promoter (which selectively inhibits transcription activator function), and/or modifying the local chromatin status (e.g., when a fusion sequence is used that modifies the target nucleic acid or modifies a polypeptide associated with the target nucleic acid). In some embodiments, the changes are transient (e.g., transcription repression or activation). In some embodiments, the changes are inheritable (e.g., when epigenetic modifications are made to the target nucleic acid or to proteins associated with the target nucleic acid, e.g., nucleosomal histones).

Non-limiting examples of fusion partners for use when targeting ssRNA target nucleic acids are include (but are not limited to): splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); heliembodiments; RNA-binding proteins; and the like. It is understood that a fusion partner can include the entire protein or in some embodiments can include a fragment of the protein (e.g., a functional domain).

In some embodiments, the heterologous sequence can be fused to the C-terminus of the Cas9 polypeptide. In some embodiments, the heterologous sequence can be fused to the N-terminus of the Cas9 polypeptide. In some embodiments, the heterologous sequence can be fused to an internal portion (i.e., a portion other than the N- or C-terminus) of the Cas9 polypeptide.

In addition the fusion partner of a chimeric Cas9 polypeptide can be any domain capable of interacting with ssRNA (which, for the purposes of this disclosure, includes intramolecular and/or intermolecular secondary structures, e.g., double-stranded RNA duplexes such as hairpins, stem-loops, etc.), whether transiently or irreversibly, directly or indirectly, including but not limited to an effector domain selected from the group comprising; Endonucleases (for example RNase I I I, the CRR22 DYW domain, Dicer, and PIN (PilT N-terminus) domains from proteins such as SMG5 and SMG6); proteins and protein domains responsible for stimulating RNA cleavage (for example CPSF, CstF, CFIm and CFIIm), Exonucleases (for example XRN-1 or Exonuclease T) Deadenylases (for example HNT3); proteins and protein domains responsible for nonsense mediated RNA decay (for example UPF1, UPF2, UPF3, UPF3b, RNP S1, Y14, DEK, REF2, and SRm160), proteins and protein domains responsible for stabilizing RNA (for example PABP) proteins and protein domains responsible for repressing translation (for example Ago2 and Ago4); proteins and protein domains responsible for stimulating translation (for example Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (for example PAP1, GLD-2, and Star-PAP) proteins and protein domains responsible for polyuridinylation of RNA (for example CI D1 and terminal uridylate transferase) proteins and protein domains responsible for RNA localization (for example from IMP1, ZBP1, She2p, She3p, and Bicaudal-D); proteins and protein domains responsible for nuclear retention of RNA (for example Rrp6); proteins and protein domains responsible for nuclear export of RNA (for example TAP, NXF1, THO, TREX, REF, and Aly) proteins and protein domains responsible for repression of RNA splicing (for example PTB, Sam68, and hnRNP A1) proteins and protein domains responsible for stimulation of RNA splicing (for example Serine/Arginine-rich (SR) domains) proteins and protein domains responsible for reducing the efficiency of transcription (for example FUS (TLS)); and proteins and protein domains responsible for stimulating transcription (for example CDK7 and HIV Tat). Alternatively, the effector domain may be selected from the group comprising Endonucleases; proteins and protein domains capable of stimulating RNA cleavage; Exonucleases; Deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription and proteins and protein domains capable of stimulating transcription. Another suitable fusion partner is a PUF RNA-binding domain, which is described in more detail in WO2012068627.

Some RNA splicing factors that can be used (in whole or as fragments thereof) as fusion partners for a Cas9 polypeptide have modular organization, with separate sequence-specific RNA binding modules and splicing effector domains. For example, members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP AI binds to exonic splicing silencers (ESSs) through its RRM domains and inhibits exon inclusion through a C-terminal Glycine-rich domain. Some splicing factors can regulate alternative use of splice site (ss) by binding to regulatory sequences between the two alternative sites. For example, ASF/SF2 can recognize ESEs and promote the use of intron proximal sites, whereas hnRNP AI can bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived postmitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple cis-elements that are located in either the core exon region or the exon extension region (i.e., between the two alternative 5′ splice sites). For more examples, see WO2010075303.

In some embodiments, a Cas9 polypeptide (e.g., a wild type Cas9, a variant Cas9, a variant Cas9 with reduced nuclease activity, etc.) can be linked to a fusion partner via a peptide spacer.

In some embodiments, a Cas9 polypeptide comprises a “Protein Transduction Domain” or PTD (also known as a CPP—cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD attached to another molecule facilitates entry of the molecule into the nucleus (e.g., in some embodiments, a PTD includes a nuclear localization signal (NLS)). In some embodiments, a Cas9 polypeptide comprises two or more NLSs, e.g., two or more NLSs in tandem. In some embodiments, a PTD is covalently linked to the amino terminus of a Cas9 polypeptide. In some embodiments, a PTD is covalently linked to the carboxyl terminus of a Cas9 polypeptide. In some embodiments, a PTD is covalently linked to the amino terminus and to the carboxyl terminus of a Cas9 polypeptide. In some embodiments, a PTD is covalently linked to a nucleic acid (e.g., a guide nucleic acid, a polynucleotide encoding a guide nucleic acid, a polynucleotide encoding a Cas9 polypeptide, etc.). Exemplary PTDs include but are not limited to a minimal undecapeptide protein transduction domain (corresponding to residues 47-57 of HIV-1 TAT comprising YGRKKRRQRRR; SEQ ID NO:56); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); an Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:52); Transportan GWTLNSAGYLLGKINLKALAALAKKIL (SEQ ID NO:53); KALAWEAKLAKALAKALAKHLAKALAKALKCEA (SEQ ID NO:54); and RQIKIWFQNRRMKWKK (SEQ ID NO:55). Exemplary PTDs include but are not limited to, YGRKKRRQRRR (SEQ ID NO:56), RKKRRQRRR (SEQ ID NO:57); an arginine homopolymer of from 3 arginine residues to 50 arginine residues; Exemplary PTD domain amino acid sequences include, but are not limited to, any of the following: YGRKKRRQRRR (SEQ ID NO:58); RKKRRQRR (SEQ ID NO:59); YARAAARQARA (SEQ ID NO:60); THRLPRRRRRR (SEQ ID NO:61); and GGRRARRRRRR (SEQ ID NO:62). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

### Type V CRISPR Systems

In other embodiments, the present disclosure provides compositions and methods using a Type V CRISPR system. The Cpf1 CRISPR systems of the present disclosure comprise i) a single endonuclease protein, and ii) a crRNA, wherein a portion of the 3′ end of crRNA contains the guide sequence complementary to a target nucleic acid. In this system, the Cpf1 nuclease is directly recruited to the target DNA by the crRNA. In some embodiments, guide sequences for Cpf1 must be at least 12nt, 13nt, 14nt, 15nt, or 16nt in order to achieve detectable DNA cleavage, and a minimum of 14nt, 15nt, 16nt, 17nt, or 18nt to achieve efficient DNA cleavage.

Cpf1 systems differ from Cas9 systems in a variety of ways. First, unlike Cas9, Cpf1 does not require a separate tracrRNA for cleavage. In some embodiments, Cpf1 crRNAs can be as short as about 42-44 bases long—of which 23-25 nt is guide sequence and 19 nt is the constitutive direct repeat sequence. In contrast, the combined Cas9 tracrRNA and crRNA synthetic sequences can be about 100 bases long.

Second, Cpf1 prefers a “TTN” PAM motif that is located 5′ upstream of its target. This is in contrast to the “NGG” PAM motifs located on the 3′ of the target DNA for Cas9 systems. In some embodiments, the uracil base immediately preceding the guide sequence cannot be substituted (Zetsche, B. et al. 2015. “Cpf1 Is a Single RNA-Guided Endonuclease of a Class 2 CRISPR-Cas System” Cell 163, 759-771, which is hereby incorporated by reference in its entirety for all purposes).

Third, the cut sites for Cpf1 are staggered by about 3-5 bases, which create “sticky ends” (Kim et al., 2016. “Genome-wide analysis reveals specificities of Cpf1 endonucleases in human cells” published online Jun. 6, 2016). These sticky ends with 3-5 bp overhangs are thought to facilitate NHEJ-mediated-ligation, and improve gene editing of DNA fragments with matching ends. The cut sites are in the 3′ end of the target DNA, distal to the 5′ end where the PAM is. The cut positions usually follow the 18th base on the non-hybridized strand and the corresponding 23rd base on the complementary strand hybridized to the crRNA.

Fourth, in Cpf1 complexes, the “seed” region is located within the first 5 nt of the guide sequence. Cpf1 crRNA seed regions are highly sensitive to mutations, and even single base substitutions in this region can drastically reduce cleavage activity (see Zetsche B. et al. 2015 “Cpf1 Is a Single RNA-Guided Endonuclease of a Class 2 CRISPR-Cas System” Cell 163, 759-771). Critically, unlike the Cas9 CRISPR target, the cleavage sites and the seed region of Cpf1 systems do not overlap. Additional guidance on designing Cpf1 crRNA targeting oligos is available on (Zetsche B. et al. 2015. “Cpf1 Is a Single RNA-Guided Endonuclease of a Class 2 CRISPR-Cas System” Cell 163, 759-771).

Persons skilled in the art will appreciate that the Cpf1 disclosed herein can be any variant derived or isolated from any source. Cpf 1 orthologs from many different species are known, including, for instance, Lachnospiraceae bacterium (e.g., ND2006), Candidatus Methanomethylophilus alvus (e.g., Mx1201), Sneatia amnii (SaCpf1), Acidaminococcus (e.g., sp. BV3L6), Parcubacteria group bacterium (e.g., GW2011); Candidatus Roizmanbacteria bacterium (e.g., GW2011), Candidatus Peregrinbacterium bacterium (e.g., GW2011), Lachnospiracea bacterium (e.g., MA2020), Btyrivibrio (e.g. sp. NC3005), Butyrivibrio fibrisolvens, Prevotella bryantii (e.g., B14), Bacteroidetes oral taxon (e.g., 274), Flavobacterium brachiophilum (e.g., FL-15), Lachnospiraceae bacterium (e.g. MC2017), Moraxella lacunata, Moraxella bovoculi (e.g., AAX08_00205), Moraxella bovoculi (e.g., AAX11_00205), Francisella novicida (e.g., U112), and Thiomicrospira (e.g., sp. XS5). Additional Cas9 orthologs can be identified using available techniques and tools. orthogonal Cas9 proteins can be selected by examining and identifying divergent repeat sequences. Tools like CRISPRfinder (Grissa et al., Nucleic Acids Res 35: W52-W57 (2007), and CRISPRdb (Grissa et al., BMC Bioinformatics 8: 172 (2007) enable identification of CRISPR arrays with their constituent spacer and repeat sequences.

In some embodiments, a complex of the present disclosure comprises a Type V CRISPR site-directed modifying polypeptide. A Type V CRISPR site-directed modifying polypeptide is also referred to herein as a “Cpf1 polypeptide.” In some embodiments, the Cpf1 polypeptide is enzymatically active, e.g., the Cpf1 polypeptide, when bound to a guide RNA, cleaves a target nucleic acid. In some embodiments, the Cpf1 polypeptide exhibits reduced enzymatic activity relative to a wild-type Cpf1 polypeptide (e.g., relative to a Cpf1 polypeptide comprising the amino acid sequence depicted in FIG. 2), and retains DNA binding activity.

The Cpf1 polypeptide can be any Cpf1 polypeptide. In some embodiments, the Cpf1 polypeptide is a naturally occurring Cpf1 polypeptide, as described above, for example, the Cpf1 peptide of SEQ ID NO:2 set forth in FIG. 2, or a Cpf1 polypeptide of any of Lachnospiraceae bacterium (e.g., ND2006), Candidatus Methanomethylophilus alvus (e.g., Mx1201), Sneatia amnii (SaCpf1), Acidaminococcus (e.g., sp. BV3L6), Parcubacteria group bacterium (e.g., GW2011); Candidatus Roizmanbacteria bacterium (e.g., GW2011), Candidatus Peregrinbacterium bacterium (e.g., GW2011), Lachnospiracea bacterium (e.g., MA2020), Btyrivibrio (e.g. sp. NC3005), Butyrivibrio fibrisolvens, Prevotella bryantii (e.g., B14), Bacteroidetes oral taxon (e.g., 274), Flavobacterium brachiophilum (e.g., FL-15), Lachnospiraceae bacterium (e.g. MC2017), Moraxella lacunata, Moraxella bovoculi (e.g., AAX08_00205), Moraxella bovoculi (e.g., AAX11_00205), Francisella novicida (e.g., U112), and Thiomicrospira (e.g., sp. XS5).

In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the amino acid sequence of any of the foregoing Cpf1 polypeptides (e.g., SEQ ID NO: 2). In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to a contiguous stretch of from 100 amino acids to 200 amino acids (aa), from 200 aa to 400 aa, from 400 aa to 600 aa, from 600 aa to 800 aa, from 800 aa to 1000 aa, from 1000 aa to 1100 aa, from 1100 aa to 1200 aa, or from 1200 aa to 1300 aa, of any of the foregoing Cpf1 polypeptides (e.g., SEQ ID NO: 2).

In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI domain of a Cpf1 polypeptide of the amino acid sequence of any of the foregoing Cpf1 polypeptides (e.g., SEQ ID NO: 2). In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCII domain of a Cpf1 polypeptide of of any of the foregoing Cpf1 polypeptides (e.g., SEQ ID NO: 2). In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCIII domain of any of the foregoing Cpf1 polypeptides (e.g., SEQ ID NO: 2).

In some embodiments, the Cpf1 polypeptide exhibits reduced enzymatic activity relative to a wild-type Cpf1 polypeptide (e.g., relative to a Cpf1 polypeptide comprising the amino acid sequence depicted in FIG. 2, SEQ ID NO: 2), and retains DNA binding activity. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the amino acid sequence of SEQ ID NO: 2; and comprises an amino acid substitution (e.g., a D→A substitution) at an amino acid residue corresponding to amino acid 917 of the amino acid sequence of SEQ ID NO: 2. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the amino acid sequence of SEQ ID NO: 2; and comprises an amino acid substitution (e.g., an E→A substitution) at an amino acid residue corresponding to amino acid 1006 of the amino acid sequence of SEQ ID NO: 2. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the amino acid sequence of SEQ ID NO: 2; and comprises an amino acid substitution (e.g., a D→A substitution) at an amino acid residue corresponding to amino acid 1255 of the amino acid sequence of SEQ ID NO: 2.

In some embodiments, the Cpf1 polypeptide is a fusion polypeptide, e.g., where a Cpf1 fusion polypeptide comprises: a) a Cpf1 polypeptide; and b) a heterologous fusion partner. In some embodiments, the heterologous fusion partner is fused to the N-terminus of the Cpf1 polypeptide. In some embodiments, the heterologous fusion partner is fused to the C-terminus of the Cpf1 polypeptide. In some embodiments, the heterologous fusion partner is fused to both the N-terminus and the C-terminus of the Cpf1 polypeptide. In some embodiments, the heterologous fusion partner is inserted internally within the Cpf1 polypeptide. Suitable heterologous fusion partners include NLS, epitope tags, fluorescent polypeptides, and the like.

In any embodiment of the invention, it is understood that the RNA-guided endonuclease can be included in the complex (or delivered to a subject) by using a nucleic acid encoding the RNA-guided endonuclease. Thus, for instance, the complex of the CRISPR system components can comprise the RNA-guided endonuclease protein itself or a nucleic acid (e.g., mRNA) encoding the protein. By delivering the nucleic acid encoding the RNA-guided endonuclease to the cell, the RNA-guided endonuclease is produced and, thus, delivered to the cell.

### Nanoparticle-Nucleic Acid Conjugates

In some embodiments, a complex of the present disclosure may further comprise a nanoparticle-nucleic acid conjugate, e.g. as described in International Patent Application No. PCT/US2016/052690. For instance, the guide RNA, donor polynucleotide, or both, can be conjugated (linked or bound) to a nanoparticle. In some embodiments, the nanoparticle is a polymer nanoparticle, which can comprise any suitable biocompatible polymer. In some embodiments, the nanoparticle is a metal nanoparticle, which can comprise any suitable metal (e.g., colloidal metal). A colloidal metal includes any water-insoluble metal particle or metallic compound dispersed in liquid water. A colloidal metal can be a suspension of metal particles in aqueous solution. Any metal that can be made in colloidal form can be used, including gold, silver, copper, nickel, aluminum, zinc, calcium, platinum, palladium, and iron. In some embodiments, gold nanoparticles are used, e.g., prepared from HAuCl₄. In some embodiments, the nanoparticles are non-gold nanoparticles that are coated with gold to make gold-coated nanoparticles.

### Nanoparticles

Nanoparticles suitable for use in a complex of the present disclosure can be any shape and can range in size from about 5 nm to about 1000 nm in size, e.g., from about 5 nm to about 75 nm, about 5 to about 50 nm, about 5 nm to about 40 nm, about 10 nm to about 30, including about 20 nm to about 30 nm in size. Nanoparticles (e.g., gold nanoparticles) suitable for use in a complex of the present disclosure can have a size in the range from about 5 nm to about 150 nm, from about 100 nm to about 500 nm, from about 500 nm to 10 μm, or from about 10 μm to about 100 μm.

A nanoparticle can comprise any suitable material, e.g., a biocompatible material. The biocompatible material can be a polymer. Suitable nanoparticle polymers include polystyrene, silicone rubber, polycarbonate, polyurethanes, polypropylenes, polymethylmethacrylate, polyvinyl chloride, polyesters, polyethers, and polyethylene. Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), and trimethylene carbonate, polyvinylpyrrolidone.

In some embodiments, the nanoparticle is a lipid nanoparticle. A lipid nanoparticle can include one or more lipids, and one or more of the polymers listed above.

In some embodiments, the nanoparticle is a colloidal metal nanoparticle. A colloidal metal includes any water-insoluble metal particle or metallic compound dispersed in liquid water. A colloid metal can be a suspension of metal particles in aqueous solution. Any metal that can be made in colloidal form can be used, including gold, silver, copper, nickel, aluminum, zinc, calcium, platinum, palladium, and iron. In some embodiments, gold nanoparticles are used, e.g., prepared from HAuCl₄. In some embodiments, the nanoparticles are non-gold nanoparticles that are coated with gold to make gold-coated nanoparticles.

In some embodiments, the nanoparticle is selected from the group consisting of a gold nanoparticle, a silver nanoparticle, a platinum nanoparticle, an aluminum nanoparticle, a palladium nanoparticle, a copper nanoparticle, a cobalt nanoparticle, an indium nanoparticle, and a nickel nanoparticle.

Methods for making colloidal metal nanoparticles, including gold colloidal nanoparticles from HAuCl₄, are known to those having ordinary skill in the art. For example, the methods described herein as well as those described elsewhere (e.g., US 2001/005581; 2003/0118657; and 2003/0053983) can be used to make nanoparticles.

Further aspects of the present disclosure include a nanoparticle, e.g., gold nanoparticle, conjugated to a nucleic acid of the CRISPR system (e.g., guide RNA, donor polynucleotide, or both). The nucleic acid can be conjugated covalently or noncovalently to the surface of the nanoparticle. For example, a nucleic acid may be covalently bonded at one end of the nucleic acid to the surface of the nanoparticle.

### Nucleic Acid Linked to a Nanoparticle

A nucleic acid (e.g., guide RNA, donor polynucleotide, or both) can be conjugated directly or indirectly to a nanoparticle surface. For example, a nucleic acid can be conjugated directly to the surface of a nanoparticle or indirectly through an intervening linker. Any type of molecule can be used as a linker. For example, a linker can be an aliphatic chain including at least two carbon atoms (e.g., 3, 4, 5, 6, 7, 8, 9, 10 or more carbon atoms), and can be substituted with one or more functional groups including ketone, ether, ester, amide, alcohol, amine, urea, thiourea, sulfoxide, sulfone, sulfonamide, and disulfide functionalities. In embodiments where the nanoparticle includes gold, a linker can be any thiol-containing molecule. Reaction of a thiol group with the gold results in a covalent sulfide (—S—) bond. Linker design and synthesis are well known in the art.

In some embodiments, the nucleic acid conjugated to the nanoparticle is a linker nucleic acid that serves to non-covalently bind one or more elements of the Type II or Type V CRISPR system (where the Type II CRISPR system comprises a Cas9 polypeptide, and a guide nucleic acid linked to a donor polynucleotide; where the Type V CRISPR system comprises a Cpf1 polypeptide, and a guide nucleic acid linked to a donor polynucleotide) to the nanoparticle-nucleic acid conjugate. For instance, the linker nucleic acid can have a sequence that hybridizes to the guide nucleic acid or donor polynucleotide.

The nucleic acid conjugated to the nanoparticle (e.g., a colloidal metal (e.g., gold) nanoparticle; a nanoparticle comprising a biocompatible polymer) can have any suitable length. When the nucleic acid is a guide nucleic acid or donor polynucleotide, the length will be as suitable for such molecules, as discussed herein and known in the art. If the nucleic acid is a linker nucleic acid, it can have any suitable length for a linker, for instance, a length of from 10 nucleotides (nt) to 1000 nt, e.g., from about 1 nt to about 25 nt, from about 25 nt to about 50 nt, from about 50 nt to about 100 nt, from about 100 nt to about 250 nt, from about 250 nt to about 500 nt, or from about 500 nt to about 1000 nt. In some instances, the nucleic acid conjugated to the nanoparticle (e.g., a colloidal metal (e.g., gold) nanoparticle; a nanoparticle comprising a biocompatible polymer) nanoparticle can have a length of greater than 1000 nt.

When the nucleic acid linked (e.g., covalently linked; non-covalently linked) to a nanoparticle comprises a nucleotide sequence that hybridizes to at least a portion of the guide nucleic acid or donor polynucleotide present in a complex of the present disclosure, it has a region with sequence identity to a region of the complement of the guide nucleic acid or donor polynucleotide sequence sufficient to facilitate hybridization. In some embodiments, a nucleic acid linked to a nanoparticle in a complex of the present disclosure has at least 50%, at least 60%, at least 70%, at least 80%, at least 90%, at least 95%, at least 98%, at least 99%, or 100%, nucleotide sequence identity to a complement of from 10 to 50 nucleotides (e.g., from 10 nucleotides (nt) to 15 nt, from 15 nt to 20 nt, from 20 nt to 25 nt, from 25 nt to 30 nt, from 30 nt to 40 nt, or from 40 nt to 50 nt) of a guide nucleic acid or donor polynucleotide present in the complex.

In some embodiments, a nucleic acid linked (e.g., covalently linked; non-covalently linked) to a nanoparticle is a donor polynucleotide, or has the same or substantially the same nucleotide sequence as a donor polynucleotide. In some embodiments, a nucleic acid linked (e.g., covalently linked; non-covalently linked) to a nanoparticle comprises a nucleotide sequence that is complementary to a donor DNA template. The nanoparticle can further comprise a nucleic acid (DNA or RNA) “barcode,” which is a short (e.g., about 5-100 nt, 5-75 nt, 5-50 nt, 5-40 nt, 5-25 nt, or 5-15 nt) sequence that is sufficiently unique as to allow the sequence to serve as a tag that can be detected by nucleic acid amplification (PCR) or other suitable methods). The barcode can be attached to the guide nucleic acid, donor nucleic acid, or linker when present, or can be a separate nucleic acid. Specific methods for creating and using nucleic acid barcodes are known in the art (see, e.g., Dahlman et al., Proc Natl Acad Sci USA.; 2017; 114(8): 2060-2065; Lyons et al., Scientific Reports, volume 7, article no. 13899 (2017)).

### Cationic Polymer and Liposomal Systems

Cationic polymers suitable for encapsulating a complex of the present invention include polycation-containing polymers that provide for enhanced escape from an endosomal compartment in a eukaryotic cell. Such polymers are referred to herein as “endosomal disruptive polymers.” A CRISPR system comprising an RNA-guided endonuclease and a guide nucleic acid linked to a donor polynucleotide, and the nucleic acid-conjugated colloidal metal nanoparticle/Type II CRISPR system complex is encapsulated in an endosomal disruptive polymer. In some embodiments, a Type II CRISPR system comprises: i) a Cas9 polypeptide; ii) a guide RNA; and iii) a donor template polynucleotide; and the nucleic acid-conjugated colloidal metal nanoparticle/Type II CRISPR system complex is encapsulated in an endosomal disruptive polymer.

In some embodiments, an endosomal disruptive polymer suitable for inclusion in a complex of the present disclosure is a cationic polymer selected from the group consisting of polyethylene imine, poly(arginine), poly(lysine), poly(histidine), poly-[2-{(2-aminoethyl)amino}-ethyl-aspartamide] (pAsp(DET)), a block co-polymer of poly(ethylene glycol) (PEG) and poly(arginine), a block co-polymer of PEG and poly(lysine), and a block co-polymer of PEG and poly{N—[N-(2-aminoethyl)-2-aminoethyl]aspartamide} (PEG-pAsp(DET)). In some embodiments, a complex of the present disclosure comprises poly{N—[N-(2-aminoethyl)-2-aminoethyl]aspartamide} (PEG-pAsp(DET)).

In some embodiments, a complex of the present disclosure further includes a silicate in the portion of the complex that encapsulates the nucleic acid-conjugated colloidal metal nanoparticle/Type II CRISPR system complex. In some embodiments, a nucleic acid-conjugated colloidal metal nanoparticle/Type II CRISPR system complex is encapsulated in alternating layers of an endosomal disruptive polymer and a silicate. In some embodiments, a nucleic acid-conjugated colloidal metal nanoparticle/Type II CRISPR system complex is encapsulated in a single layer of an endosomal disruptive polymer. In some embodiments, a nucleic acid-conjugated colloidal metal nanoparticle/Type II CRISPR system complex is encapsulated in two or more layer of an endosomal disruptive polymer.

Cationic liposomes suitable for encapsulating a complex of the present invention include ({2,2-bis[(9Z,12Z)-Octadeca-9,12-dien-1-yl]-1,3-dioxan-5-yl}methyl) dimethylamine; (3aR,5s,6aS)-N,N-dimethyl-2,2-di((9Z,12Z)-octadeca-9,12-dien-1-yl)tetrahydro-3aH-cyclopenta[d][1,3]dioxol-5-amine, (3aR,5r,6aS)-N,N-dimethyl-2,2-di((9Z,12Z)-octadeca-9,12-dien-1-yl)tetrahydro-3aH-cyclopenta[d][1,3]dioxol-5-amine; (3aR,5R,7aS)-N,N-dimethyl-2,2-di((9Z,12Z)-octadeca-9,12-dien-1-yl)hexahydrobenzo[d][1,3]dioxol-5-amine, (3aS,5R,7aR)-N,N-dimethyl-2,2-di((9Z,12Z)-octadeca-9,12-dien-1-yl)hexahydrobenzo[d][1,3]dioxol-5-amine, (2-{2,2-bis[(9Z,12Z)-Octadeca-9,12-dien-1-yl]-1,3-dioxan-4-yl}ethyl)dimethylamine, (3aR,6aS)-5-methyl-2-((6Z,9Z)-octadeca-6,9-dien-1-yl)-2-((9Z,12Z)-octadeca-9,12-dien-1-yl)tetrahydro-3aH-[1,3]dioxolo[4,5]pyrrole, (3aS,7aR)-5-methyl-2,2-di((9Z,12Z)-octadeca-9,12-dien-1-yl)hexahydro-[1,3]dioxolo[4,5-c]pyridine, (3aR,8aS)-6-methyl-2,2-di((9Z,12Z)-octadeca-9,12-dien-1-yl)hexahydro-3aH-[1,3]dioxolo[4,5-d]azepine, (6Z,9Z,28Z,31Z)-heptatriaconta-,9,28,31-tetraen-19-yl 2-(dimethylamino)acetate, (6Z,9Z,28Z,31Z)-heptatriaconta-6,9,28,31-tetraen-19-yl 3-(dimethylamno)propanoate, [6Z, 9Z,28Z,31Z)-heptatriaconta-6,9,28,31-tetraen-19-yl-4-(dimethylamno)butanoate]; (6Z,9Z,28Z,31Z)-heptatriaconta-6,9,28,31-tetraen-19-yl 5-(dimethylamino)pentanoate; (6Z,9Z,28Z,31Z)-heptatriaconta-6,9,28,31-tetraen-19-yl 6-(dimethylamino)hexanoate, (3-{2,2-bis[(9Z,12Z)-Octadeca-9,12-dien-1-yl]-1,3-dioxan-4-yl}propyl)dimethylamine, 1-((3aR,5r,6aS)-2,2-di((9Z,12Z)-octadeca-9,12-dien-1-yl)tetrahydro-3aHcyclopenta[d][1,3]dioxol-5-yl)-N,N-dimethylmethanamine, 1-((3aR,5s,6aS)-2,2-di((9Z,12Z)-octadeca-9,12-dien-1-yl)tetrahydro-3aHcyclopenta[d][1,3]dioxol-5-yl)-N,N-dimethylmethanamine, 8-methyl-2,2-di((9Z,12Z)-octadeca-9,12-dien-1-yl)-1,3-dioxa-8-azaspiro[4.5]decane, 2-(2,2-di((9Z,12Z)-octadeca-9,12-dien-1-yl)-1,3-dioxolan-4-yl)-N-methyl-N-(pyridin-3-ylmethyl)ethanamine 1,3-bis(9Z,12Z)-Octadeca-9,12-dien-1-yl 2-[2-(dimethylamino)ethyl]propanedioate N,N-dimethyl-1-((3aR,5R,7aS)-2-((8Z,11Z)-octadeca-8,11-dien-1-yl)-2-((9Z,12Z)-octadeca-9,12-dien-1-yl)hexahydrobenzo[d][1,3]dioxol-5-yl)methanamine, N,N-dimethyl-1-((3aR,5S,7aS)-2-((8Z,11Z)-octadeca-8,11-dien-1-yl)-2-((9Z,12Z)-octadeca-9,12-dien-1-yl)hexahydrobenzo[d][1,3]dioxol-5-yl)methanamine, (1s,3R,4S)—N,N-dimethyl-3,4-bis((9Z,12Z)-octadeca-9,12-dien-1-yloxy)cyclopentan amine; (1s,3R,4S)—N,N-dimethyl-3,4-bis((9Z,12Z)-octadeca-9,12-dien-1-yloxy)cyclopentan amine; 2-(4,5-di((8Z,11Z)-heptadeca-8,11-dien-1-yl)-2-methyl-1,3-dioxolan-2-yl)-N,N-dimethylethanamine, 2,3-di((8Z,11Z)-heptadeca-8,11-dien-1-yl)-N,N-dimethyl-1,4-dioxaspiro[4.5] decan-8-amine; (6Z,9Z,28Z,31Z)-heptatriaconta-6,9,28,31-tetraen-19-yl 4-(diethylamino)butanoate, (6Z,9Z,28Z,31Z)-heptatriaconta-6,9,28,31-tetraen-19-yl 4-[bis(propan-2-yl)amino]butanoate, N-(4-N,N-dimethylamino)butanoyl-(6Z,9Z,28Z,31Z)-heptatriaconta-6,9,28,31-tetraen-19-amine, (2-{2,2-bis[(9Z,12Z)-Octadeca-9,12-dien-1-yl]-1,3-dioxan-5-yl}ethyl)dimethylamine, (4-{2,2-bis[(9Z,12Z)-Octadeca-9,12-dien-1-yl]-1,3-dioxan-5-yl}butyl)dimethylamine, (6Z,9Z,28Z,31Z)-heptatriaconta-6,9,28,31-tetraen-19-yl(2-(dimethylamino)ethyl)carbamate, 2-(dimethylamino)ethyl (6Z,9Z,28Z,31Z)-heptatriaconta-6,9,28,31-tetraen-19-ylcarbamate, (6Z,9Z,28Z,31Z)-heptatriaconta-6,9,28,31-tetraen-19-yl 3-(ethylamino)propanoate, (6Z,9Z,28Z,31Z)-heptatriaconta-6,9,28,31-tetraen-19-yl 4-(propan-2-ylamino) butanoate; N1,N1,N2-trimethyl-N2-((11Z,14Z)-2-((9Z,12Z)-octadeca-9,12-dien-1-yl)icosa-11,14-dien-1-yl)ethane-1,2-diamine, 3-(dimethylamino)-N-((11Z,14Z)-2-((9Z,12Z)-octadeca-9,12-dien-1-yl)icosa-11,14-dien-1-yl)propanamide, (6Z,9Z,28Z,31Z)-heptatriaconta-6,9,28,31-tetraen-19-yl 4-(methylamino)butanoate, Dimethyl({4-[(9Z,12Z)-octadeca-9,12-dien-1-yloxy]-3-{[(9Z,12Z)-octadeca-9,12-dien-1-yloxy]methyl}butyl})amine, 2,3-di((8Z,11Z)-heptadeca-8,11-dien-1-yl)-8-methyl-1,4-dioxa-8-azaspiro[4.5]decane, 3-(dimethylamino)propyl (6Z,9Z,28Z,31Z)-heptatriaconta-6,9,28,31-tetraen-19-ylcarbamate, 2-(dimethylamino)ethyl ((11Z,14Z)-2-((9Z,12Z)-octadeca-9,12-dien-1-yl)icosa-11,14-dien-1-yl)carbamate, 1-((3aR,4R,6aR)-6-methoxy-2,2-di((9Z,12Z)-octadeca-9,12-dien-1-yl)tetrahydrofuro[3,4-d][1,3]dioxol-4-yl)-N,N-imethylmethanamine, (6Z,9Z,28Z,31Z)-heptatriaconta-6,9,28,31-tetraen-19-yl 4-[ethyl(methyl)amino]butanoate, 6Z,9Z,28Z,31Z)-heptatriaconta-6,9,28,31-tetraen-19-yl 4-aminobutanoate, 3-(dimethylamino)propyl ((11Z,14Z)-2-((9Z,12Z)-octadeca-9,12-dien-1-yl)icosa-11,14-dien-1-yl)carbamate, 1-((3aR,4R,6aS)-2,2-di((9Z,12Z)-octadeca-9,12-dien-1-yl)tetrahydrofuro[3,4-d][1,3]dioxol-4-yl)-N,N-dimethylmethanamine, (3aR,5R,7aR)-N,N-dimethyl-2,2-di((9Z,12Z)-octadeca-9,12-dien-1-yl)hexahydrobenzo[d][1,3]dioxol-5-amine, (11Z,14Z)—N,N-dimethyl-2-((9Z,12Z)-octadeca-9,12-dien-1-yl)icosa-11,14-dien-1-amine, (3aS,4S,5R,7R,7aR)-N,N-dimethyl-2-((7Z,10Z)-octadeca-7,10-dien-1-yl)-2-((9Z,12Z)-octadeca-9,12-dien-1-yl)hexahydro-4,7-methanobenzo[d][1,3]dioxol-5-amine, N,N-dimethyl-3,4-bis((9Z,12Z)-octadeca-9,12-dien-1-yloxy)butan-1-amine, 3-(4,5-di((8Z,11Z)-heptadeca-8,11-dien-1-yl)-1,3-dioxolan-2-yl)-N,N-dimethylpropan-1-amine.

### Methods of Preparation

The present disclosure provides methods of making a modified guide nucleic acid, a guide nucleic acid covelantly or non-covelantly linked to a donor nucleic acid, complex of the present disclosure.

The guide and donor nucleic acids described herein can be prepared by any suitable technique, including well known recombinant methods as well as nucleic acid synthesis. Moreover, conjugated RNA-DNA (e.g., guide nucleic acid and donor DNA) can be synthesized directly. Synthesis of both DNA and RNA can be accomplished using solid-phase synthesis; thus, RNA-DNA can be synthesized with a single nucleic acid reaction step. Alternatively, a guide nucleic acid and donor nucleic acid can be produced separately and linked, such as through a chemical linkage (e.g., click chemistry or other suitable reaction) or hybridization. Functionalizing nucleic acids with chemical functional groups can be performed using known techniques.

Other aspects of making and using the various compositions are as described below.

### Methods of Making a Complex

Further aspects of the present disclosure include a method of making a complex of the present disclosure. In some embodiments, the nanoparticle is functionalized with a sulfur (e.g., a thiol moiety), and the nucleic acid is attached to the nanoparticle via the sulfur (e.g., via the thiol moiety). Once the nucleic acid is attached to the nanoparticle, the Type II site directed DNA modifying polypeptide (e.g., Cas9 polypeptide) or the Type V site directed DNA modifying polypeptide (e.g., Cpf1 polypeptide) and the guide nucleic acid are contacted with the nucleic acid-nanoparticle conjugate, to form a complex of the present disclosure.

An implementation of the method may include loading a gold nanoparticle (GNP) conjugated to DNA via a thiol group with a Cas9/gRNA ribonucleoprotein (RNP) to produce a Cas9 RNP-DNA-GNP complex. The GNP-DNA conjugate may be produced by reacting a GNP with a DNA-thiol. The GNP may have a diameter of about 30 nm. In some embodiments, the GNP-DNA conjugate is hybridized with a donor single-stranded DNA before loading the Cas9 RNP. After forming the Cas9 RNP-DNA-GNP complex, the complex may be coated with silicate and an endosomal disruptive polymer, such as a pAsp(DET) polymer to form an encapsulated Cas9 RNP-DNA-GNP complex.

### Method of Binding a Target Nucleic Acid and Methods of Modifying a Target Nucleic Acid

The present disclosure provides methods of binding a target nucleic acid present in a eukaryotic cell. The methods generally involve contacting a eukaryotic cell comprising a target nucleic acid with a complex of the present disclosure, wherein the complex enters the cell, and wherein the guide nucleic acid and site-directed DNA-modifying polypeptide (e.g., a Cas9 polypeptide or a Cpf1 polypeptide) (and, if present, a donor polynucleotide) are released from the complex in an endosome in the cell. Once released from the endosome, the guide nucleic acid and site-directed DNA-modifying polypeptide (e.g., a Cas9 polypeptide or a Cpf1 polypeptide) (and, if present, a donor polynucleotide) can bind a target nucleic acid, e.g., where the target nucleic acid is in the nucleus, in a mitochondrion, or in the cytoplasm. In some case, the cell is in vitro or the cell is ex vivo (e.g., the method is performed ex vivo, wherein the cell (optionally autologous to a patient) is treated outside the body of a patient, and then introduced into the patient, optionally after culturing). In some embodiments, the cell is in vivo. In some embodiments, the cell is present in a multicellular organism. In some embodiments, where the complex comprises a dead Cas9 polypeptide, the dead Cas9 polypeptide modulates transcription from the target nucleic acid. In some embodiments, e.g., where the complex comprises a Cas9 fusion polypeptide, the Cas9 fusion polypeptide modifies the target nucleic acid. In some embodiments, where the complex comprises a Cas9 polypeptide, the Cas9 polypeptide cleaves the target nucleic acid. In some embodiments, where the complex comprises a Cpf1 polypeptide, the Cpf1 polypeptide cleaves the target nucleic acid.

As noted above, in some embodiments, the complex comprises a donor template polynucleotide. In these instances, the method comprises contacting the target nucleic acid with the donor template polynucleotide. In some embodiments, the donor polynucleotide (e.g., a DNA repair template) replaces at least a portion of a target nucleic acid, e.g., to repair a defect in the target nucleic acid.

The present disclosure provides methods of genetically modifying a eukaryotic target cell. The methods generally involve contacting the eukaryotic target cell with a complex of the present disclosure. The complex enters the cell, and the guide RNA, site-directed DNA-modifying polypeptide (e.g., a Cas9 polypeptide or a Cpf1 polypeptide), and donor polynucleotide are released from the complex in an endosome in the cell. Once released from the endosome, the guide nucleic acid and site-directed DNA-modifying polypeptide (e.g., a Cas9 polypeptide or a Cpf1 polypeptide) (and, if present, a donor polynucleotide) can bind a target nucleic acid, e.g., where the target nucleic acid is in the nucleus, in a mitochondrion, or in the cytoplasm. In some case, the cell is in vitro. In some embodiments, the cell is in vivo. In some embodiments, the cell is present in a multicellular organism. In some embodiments, the target cell is an insect cell. In some embodiments, the target cell is an arachnid cell. In some embodiments, the target cell is a cell of or in an invertebrate. In some embodiments, the target cell is a protozoan cell. In some embodiments, the target cell is a plant cell. In some embodiments, the target cell is present in a plant or a plant tissue. In some embodiments, the target cell is an animal cell. In some embodiments, the target cell is present in an animal, e.g., a human, or a non-human animal. In some embodiments, the target cell is a mammalian cell. In some embodiments, the target cell is present in a mammal, e.g., in a human or a non-human mammal. In some embodiments, is a myoblast, a neuron, a chondrocyte, a lymphocyte, an epithelial cell, an adipocyte, or a keratinocyte. In some embodiments, the target cell is pluripotent cell. In some embodiments, the target cell is a stem cell, e.g., an embryonic stem cell, a neuronal stem cell, a hematopoietic stem cell, an adult stem cell, an induced stem cell, etc.

A method of the present disclosure can be used in combination with one or more other methods of delivering a Type II or Type V CRISPR system to a eukaryotic cell. For example, in some embodiments, a method of the present disclosure for genetically modifying a eukaryotic target cell comprises administering to an individual in need thereof a complex of the present disclosure; and administering a recombinant vector comprising a nucleotide sequence encoding one or more components of a Type II or Type V CRISPR system (e.g., a nucleotide sequence encoding a Cas9 polypeptide; a nucleotide sequence encoding a Cpf1 polypeptide; a nucleotide sequence encoding a guide RNA). As another example, in some embodiments, a method of the present disclosure for genetically modifying a eukaryotic target cell comprises administering to an individual in need thereof a complex of the present disclosure; and administering an RNA comprising a nucleotide sequence encoding one or more components of a Type II or Type V CRISPR system (e.g., a nucleotide sequence encoding a Cas9 polypeptide; a nucleotide sequence encoding a Cpf1 polypeptide; a nucleotide sequence encoding a guide RNA).

### Target Cells of Interest

In some of the above applications, the subject methods may be employed to induce target nucleic acid cleavage, target nucleic acid modification, and/or to bind target nucleic acids (e.g., for visualization, for collecting and/or analyzing, etc.) in mitotic or post-mitotic cells in vivo and/or ex vivo and/or in vitro (e.g., to disrupt production of a protein encoded by a targeted mRNA). Because the guide nucleic acid provides specificity by hybridizing to target nucleic acid, a mitotic and/or post-mitotic cell of interest in the disclosed methods may include a cell from any eukaryotic cell or organism (e.g. a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens, C. agardh, and the like, a fungal cell (e.g., a yeast cell), an animal cell, a cell from an invertebrate animal (e.g. fruit fly, cnidarian, echinoderm, nematode, an insect, an arachnid, etc.), a cell from a vertebrate animal (e.g., fish, amphibian, reptile, bird, mammal), a cell from a mammal, a cell from a rodent, a cell from a human, etc.), or a protozoan cell.

Any type of cell may be of interest (e.g. a stem cell, e.g. an embryonic stem (ES) cell, an induced pluripotent stem (iPS) cell, a germ cell; a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e. splittings, of the culture. For example, primary cultures are cultures that may have been passaged 0 times, 1 time, 2 times, 4 times, 5 times, 10 times, or 15 times, but not enough times go through the crisis stage. In some embodiments, the primary cell lines are maintained for fewer than 10 passages in vitro. Target cells are in some embodiments unicellular organisms, or are grown in culture.

If the cells are primary cells, they may be harvest from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. are most conveniently harvested by biopsy. An appropriate solution may be used for dispersion or suspension of the harvested cells. Such solution will generally be a balanced salt solution, e.g. normal saline, phosphate-buffered saline (PBS), Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells may be used immediately, or they may be stored, frozen, for long periods of time, being thawed and capable of being reused. In such embodiments, the cells will usually be frozen in 10% or more DMSO, 50% or more serum, and about 40% buffered medium, or some other such solution as is commonly used in the art to preserve cells at such freezing temperatures, and thawed in a manner as commonly known in the art for thawing frozen cultured cells.

In some embodiments, a method of modifying a target nucleic acid comprises homology-directed repair (HDR). In some embodiments, use of a complex of the present disclosure to carry out HDR provides an efficiency of HDR of at least 1%, at least 2%, at least 3%, at least 4%, at least 5%, at least 10%, at least 15%, at least 20%, at least 25%, or more than 25%.

In some embodiments, a method of modifying a target nucleic acid comprises non-homologous end joining (NHEJ). In some embodiments, use of a complex of the present disclosure to carry out HDR provides an efficiency of NHEJ of at least 1%, at least 2%, at least 3%, at least 4%, at least 5%, at least 10%, at least 15%, at least 20%, at least 25%, or more than 25%.

### Utility

Methods of the present disclosure for binding and/or modifying a target nucleic acid in a eukaryotic cell are useful in a variety of therapeutic and research applications, including site directed DNA recombination for genome editing, gene inactivation, transcriptional attenuation and transcriptional enhancement.

Methods of the present disclosure for binding and/or modifying a target nucleic acid in a eukaryotic cell are useful for carrying out non-homologous end joining or homology-directed repair. Thus, for example, a method of the present disclosure for modifying a target nucleic acid in a eukaryotic cell is useful for modifying the genome of the cell, e.g., in the context of treating a disease caused by a mutation in the genome

### Kits

The present disclosure provides a kit for carrying out a method of the present disclosure.

In some embodiments, a kit of the present disclosure comprises a complex comprising: a) a nanoparticle-nucleic acid conjugate; a Type II or a Type V CRISPR system comprising a site-directed DNA-modifying polypeptide and a guide RNA, and optionally also comprising a donor polynucleotide (e.g., a DNA donor template); and b) a polycation-based endosomal escape polymer. In some embodiments, a kit includes a recombinant expression vector that provides for in vitro production of a guide RNA.

In some embodiments, a kit of the present disclosure comprises a complex comprising: a) a nanoparticle-nucleic acid conjugate; a Cas9 polypeptide; and a guide RNA; and b) a polycation-based endosomal escape polymer. In some embodiments, a kit of the present disclosure comprises a complex comprising: a) a nanoparticle-nucleic acid conjugate; a Cpf1 polypeptide; and a guide RNA; and b) a polycation-based endosomal escape polymer. In some embodiments, a kit includes a recombinant expression vector that provides for in vitro production of a guide RNA.

In some embodiments, a kit of the present disclosure comprises a complex comprising: a) a nanoparticle-nucleic acid conjugate; a Cas9 polypeptide; a guide RNA; and a donor DNA; and b) a polycation-based endosomal escape polymer. In some embodiments, a kit of the present disclosure comprises a complex comprising: a) a nanoparticle-nucleic acid conjugate; a Cpf1 polypeptide; a guide RNA; and a donor DNA; and b) a polycation-based endosomal escape polymer. In some embodiments, a kit includes a recombinant expression vector that provides for in vitro production of a guide RNA.

In some embodiments, a kit of the present disclosure includes a colloidal metal nanoparticle conjugated to a nucleic acid. In some embodiments, a kit of the present disclosure includes: a) a colloidal metal nanoparticle conjugated to a nucleic acid; and b) a Cas9 polypeptide. In some embodiments, a kit of the present disclosure includes: a) a colloidal metal nanoparticle conjugated to a nucleic acid; b) a Ca9 polypeptide; and c) a guide RNA. In some embodiments, a kit includes a recombinant expression vector that provides for in vitro production of a guide RNA.

A kit of the present disclosure can include one or more additional components, e.g., a buffer, a nuclease inhibitor, a protease inhibitor, and the like. A kit of the present disclosure can include a positive control and/or a negative control.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Screening Method

The invention also comprises a method of screening test compounds for the ability to enhance the gene-editing activity of the RNA-guided endonuclease. The compound might enhance the gene-editing activity of the RNA-guided endonuclease if it enhances the gene-editing process in any way, such as by improving the delivery of the RNA-guided endonuclease (e.g., uptake, cell targeting, endosomal escape); improving the interaction between the RNA-guided endonuclease with the guide RNA or tracer RNA (or single guide RNA); improving interaction between the guide RNA/RNA-guided endonuclease complex with the target DNA; improving cleavage the target DNA by the RNA-guided endonuclease; improving repair of the DNA following cleavage, or improving the integration of donor DNA into the repair site.

The method comprises linking a test compound to a guide RNA; and combining (i) the guide RNA linked to the test compound; (ii) an RNA guided endonuclease; (iii) a target DNA; and optionally (iv) a donor polynucleotide (donor DNA) or template DNA. The method further comprises selecting the test compound as enhancing the activity of the RNA-guided endonuclease if the guide RNA linked to the test compound produces enhanced gene editing of the target DNA as compared to the guide RNA without the test compound. Enhanced gene editing, as used herein, encompasses any improvement (e.g., specificity, efficiency) in the gene editing, for example, increase in DNA targeting specificity, decrease in off-target effects, and/or increased efficiency of NHEJ/HDR.

The test compound can be linked to the guide RNA by any suitable method. Thus, for instance, the guide RNA can be modified as described herein to comprise a functional group at the 5′ or 3′ terminus, and the test compound can be linked to the functional group. For example, the test compound can comprise or be modified to comprise a functional group (e.g., azide, tetrazine, alkyne, strained alkyne, or strained alkene) that reacts with a functional group on the guide RNA described herein. In one particular embodiment, the guide RNA comprises an azide or tetrazine at the 5′ or 3′ terminus, and the test compound comprises an alkyne, strained alkyne, or strained alkene, as appropriate, so that the test compound links to the functional group of the guide RNA through cycloaddition, providing a linkage comprising a triazole or cyclic alkene group between the guide RNA and test compound. Of course, the opposite order of groups also can be used, i.e., the guide RNA can comprise an alkyne, strained alkyne, or strained alkene at the 5′ or 3′ terminus, and the test compound can comprise an azide or tetrazine, as appropriate, so that the test compound links to the functional group of the guide RNA through cycloaddition.

The method can further comprise generating a library of test compounds. The library of test compounds can each comprise or be modified to comprise a functional group (e.g., azide, tetrazine, alkyne, strained alkyne, or strained alkene) that reacts with the functional group of the linker of the guide RNA as described herein. For instance, the library compound can comprise an azide group that reacts with a strained alkyne (e.g., DBCO) on the guide RNA, or the library compound can comprise a strained alkyne (e.g., DBCO) group that reacts with an azide group on the guide RNA. Other matched groups can be used that react to link the compounds through a cycloaddition reaction, examples of which are provided herein. As part of the screening method, each test compound can be linked to the guide RNA just before screening. Alternatively, the method can comprise generating a library of test compounds each of which is already linked to guide RNA, such that the library is ready for testing. In one embodiment, each test compound is linked to a guide RNA by way of a linkage comprising a triazole or cyclic alkene group.

The method is not limited to any particular type of molecule. Any test compound that can be linked to the guide RNA can be used. Thus, for instance, the test compound can be a small molecule, peptide, or nucleic acid. Similarly, the test compound libraries can be libraries of small molecules, peptides, or nucleic acids.

The method can be performed as a cell-free biochemical assay, or as a cell-based assay. When performed as a cell-free assay, the components of the system can be combined in an appropriate aqueous buffer solution. The conditions of the solution can be chosen to mimic the desired physiological conditions. For instance, the pH of the solution can be controlled or even varied to mimic the conditions of the endosome or the interior of the cell, or some sequence of such environments.

When performed as a cell-based assay, the step of combining (i) the guide RNA linked to the test compound; (ii) an RNA-guided endonuclease; (iii) a target DNA; and optionally (iv) a donor DNA can be performed by administering the guide RNA linked to the test compound, the RNA guided endonuclease, and, optionally, the donor DNA to a cell comprising the target DNA. Administration can be accomplished by any suitable technique. In some instances, it may be desirable to contact the cells with the components of the assay, above, in a manner that allows endosomal delivery to the interior of the cell. In the cell-cell based assay, the test compound is selected as enhancing the activity of the RNA-guided endonuclease if the guide RNA linked to the test compound produces enhanced gene editing in the cell as compared to the guide RNA without the test compound.

The guide RNA linked to the test compound, the RNA guided endonuclease, and, optionally, the donor DNA can be combined with target DNA (or administered to a cell in a cell based assay) together or separately. For instance, the donor DNA can be linked to the modified endonuclease. Also, the guide RNA (e.g., single guide RNA) can be linked to the donor RNA, when present.

Whether performed as a cell-free or cell-based assay, the method can be performed in a high-throughput format. Any of a wide variety of high-throughput assay formats known in the art can be used. For instance, the screening can be performed by combining the guide RNA linked to the test compound, the RNA guided endonuclease, and, optionally, the donor DNA in the wells of a multi-well plate. Each well can comprise a different test compound linked to the guide RNA. The use of multi-well assay plates allows for the parallel processing and analysis of multiple samples. Multi-well assay plates (also known as microplates or microtiter plates) can take a variety of forms, sizes and shapes (for instance, round- or flat-bottom multi-well plates). Non-limiting examples of multi-well plate formats include, for instance, 96-well plates (e.g., 12×8 array of wells), 384-well plates (e.g., 24×16 array of wells), 1536-well plate (e.g., 48×32 array of well), 3456-well plates, and even 9600-well plates. Alternatively, the assays can be performed in high-throughput microfluidic devices, some of which enable single-cell culture and sorting.

Methods of detecting enhanced gene editing are known in the art. For example, reporter genes (e.g., fluorescent reporter genes) can be used as a positive or negative marker indicating whether gene editing has been successful. For instance, a cell line expressing a first type of reporter (e.g., gene blue-fluorescent protein (BFP)) can be screened for BFP knockout (i.e., loss of fluorescence) to measure NHEJ efficiency, or screened for expression of a second, different type of reporter (e.g., green fluorescent protein (GFP)) in place of the first reporter to measure HDR efficiency.

Gene Editing with Enrichment

Also provided herein is a method of editing the genes of a cell that provides for enrichment of the cell population for those cells that are most likely to incorporate a donor nucleic acid. The comprises (a) administering an RNA guided endonuclease, a guide RNA, and, optionally, donor nucleic acid to a cell comprising target DNA to be edited, wherein the guide RNA and/or donor nucleic acid, when present, comprises a detectable label; (b) selecting cells by detecting the detectable label; and (c) culturing the selected cells.

Any suitable detectable label can be used. A wide variety of detectable labels are known in the art that can be used in accordance with the invention. In one embodiment, the detectable label is fluorescent label.

When the guide RNA comprises the detectable label, the label can be attached to the guide RNA at any position, for instance, the 3′ or 5′ terminus. In one embodiment, the guide RNA is a Cas9 single guide RNA or crRNA, and the label is positioned at the 5′ terminus. In another embodiment, the guide RNA is a Cpf1 guide RNA, and the label is positioned at the 3′ terminus.

Similarly, when a donor nucleic acid is used, the donor nucleic acid can be modified with the detectable label at any position, for instance, the 3′ or 5′ terminus. Furthermore, both the guide RNA and donor nucleic acid can comprise a detectable label, which can be the same or different.

In another embodiment, the donor nucleic is covalently linked to the guide RNA, and the linked guide RNA/donor nucleic acid is labeled at the either or both ends of the linked construct. By way of non-limiting examples, the guide RNA can be a Cas9 single guide RNA or crRNA linked to a donor nucleic acid at the 5′ terminus of the guide RNA or crRNA, and the detectable label can be positioned between the guide RNA or crRNA and the donor nucleic acid, or the detectable label can be positioned at the 5′ terminus of the donor nucleic acid. Similarly, the guide RNA can be a Cpf1 guide RNA linked to the donor nucleic acid at the 3′ terminus, and the label can be positioned between the guide RNA and the donor nucleic acid, or the detectable label can be positioned at the 3′ terminus of the donor nucleic acid.

In yet another embodiment, the donor nucleic acid can be linked to the RNA-guided endonuclease, with or without a detectable label.

The label can be detected and, optionally, separated or sorted from cells without the detectable label by any suitable method. One well-known method that can be used for this purpose is fluorescence activated cell sorting (FACS).

The cells having the detectable label provide a cell population that is enriched for the components needed for gene editing. Furthermore, as demonstrated by the inventors, the presence of the detectable labels on the guide RNA and/or donor DNA do not prevent or substantially impair the guide RNA and/or donor RNA, or other components of the system, from performing the gene editing functions. The cells thus separated and enriched can then be cultured to provide a rapid and efficient method of editing the genes of the cells.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1. Extended gRNA Linkages

This experiment investigated whether gRNA sequence can be engineered for CRISPR/Cas9 genome editing applications. Currently used gRNA is composed of sequences that are all necessary for Cas9 activity to hybridize with donor DNA. We also investigated the effect of changing the size and charge of the gRNA. Adding more bases to the gRNA increases the molecular weight and negative charge of the gRNA, which are important factors that affect particle formation. Variation in size and charge can affect the future delivery technologies. Lipid nanoparticles and polymer nanoparticles are sensitive to size and charge changes as many of cationic molecules bind to Cas9 RNP with electrostatic interactions. Lastly, the addition of bases to the 3′ end can increase the half-life of functionally important gRNA sequence. Importantly, additional sequences can be used to hybridize to donor DNA, which works like a functional group for chemistry.

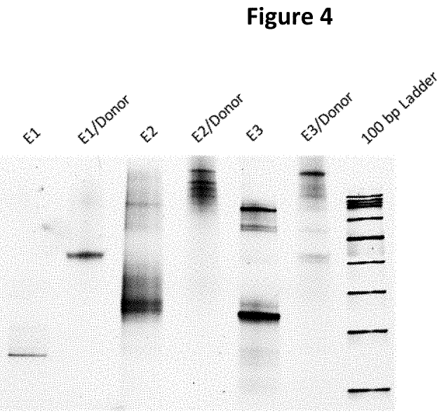

Several designs (20 base extension_S1, 20 base extension_S2, 40 base extension_S3) were tested. Three gRNAs with extended sequence have from about 120 to 140 nt size. gRNA_E1 has an extended sequence at the 3′ end that hybridizes with the 3′ end of Donor DNA. gRNA_E2 has an extended sequence at the 3′ end that hybridizes with the 5′ end of Donor DNA. gRNA_E3 has a repeated extended sequences at the 3′ end that hybridizes with the 3′ end of up to two Donor DNAs. gRNA_E4 has an extended sequence at the 3′ end that binds to bridge DNA (Green). The bridge DNA also binds to the 5′ end of Donor DNA and connects gRNA_E4 and Donor DNA. FIG. 3 illustrates the extended gRNA designs. Each extended gRNA is hybridized to Donor DNA and then analyzed using gel electrophoresis (FIG. 4). Extended gRNAs were hybridized with Donor DNA or bridge DNA and Donor DNA with heat denaturation and rehybridization. The hybridized strands were purified with 300 kDa concentrator. FIG. 4 shows a clear shift of the hybridized gRNAs.

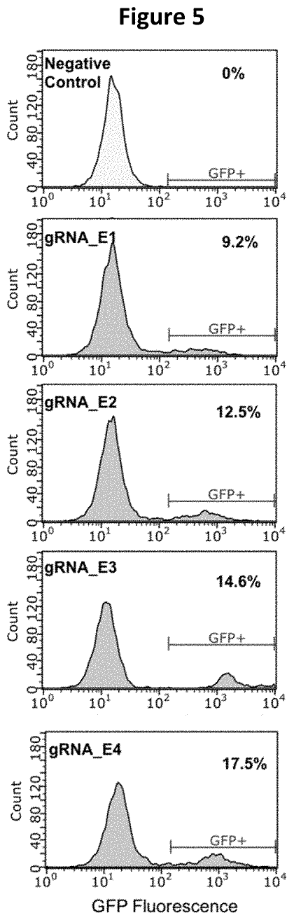

All of them showed intact Cas9 activity, as shown by in vitro cleavage assay [data not shown]. Then nucleofection was conducted to check the knock-out of BFP in BFP expressing human embryonic kidney (BFP-HEK) cells. GFP has only one amino acid difference from BFP. Cas9 complexed to gRNA targets the sequence and Donor DNA converts BFP gene into GFP gene via HDR. BFP-HEK cells were nucleofected with extended gRNA hybridized to Donor DNA complexed together with Cas9 protein. Cells were analyzed with flow cytometry 3 days after the transfection. GFP+ population percentage is quantified with flow cytometry analysis software InCyte, a representative result of triplicate experiment samples. GFP population generation via Cas9 mediated homology directed repair (HDR) shows efficient HDR with extended gRNA designs (FIG. 5).

Finally, particle delivery was conducted with Donor DNA hybridized to the gRNA. We used polymer nanoparticle with gold nanoparticle core, which is similar to CRISPR-Gold (PCT/US2016/052690). The particle delivered extended gRNA-Donor DNA and Cas9 into cells and induced efficient HDR (about 10% GFP+ population) (data not shown).

Accordingly, the four non-covalent linkage designs include direct gRNA-donor DNA hybridization and gRNA-bridge DNA-donor DNA hybridization. The direct gRNA-donor DNA hybridization was confirmed with gel electrophoresis. The BFP-HEK cell treatment and flow cytometry experiments clearly show efficient HDR with extended gRNA designs. Among the extended gRNA designs, gRNA_E4 shows the highest efficiency.

### Example 6: crRNA Modification for Cpf1

Above, we showed how crRNA for Cas9 is conjugated to Donor DNA. We also investigated whether crRNA for Cpf1 can be modified in a similar method.

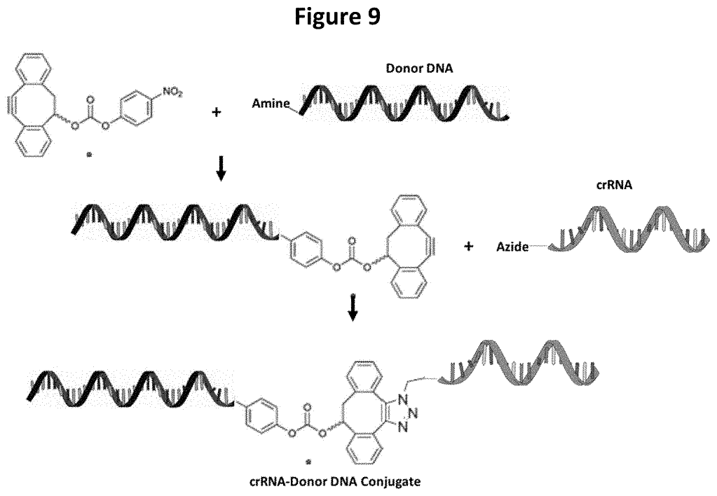

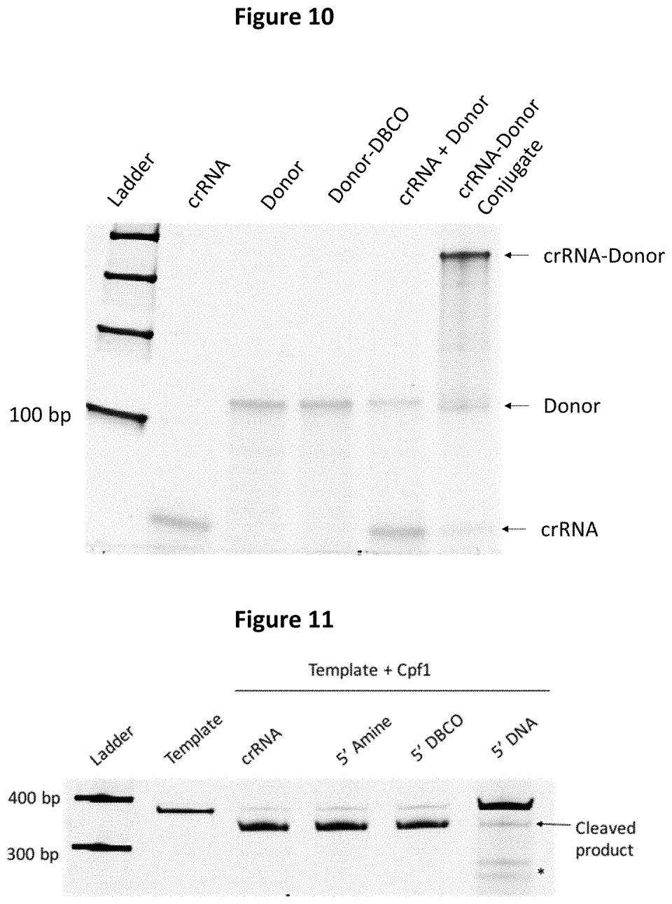

FIG. 9 illustrates the chemical conjugation of crRNA (Cpf1) and donor DNA as exemplified herein. crRNA was purchased with azide modification on its end and donor DNA was purchased with amine modification. Activated p-nitrophenyl carbonate reacts with the amine on the donor DNA. After purification, the product was mixed with crRNA with azide modification on its end. crRNA-DNA conjugation is purified by gel extraction after the reaction. FIG. 10 shows that donor DNA with DBCO and crRNA with azide conjugate successfully. Gel electrophoretic separation confirming Cpf1 activity of chemically modified Cpf1 crRNAs is provided in FIG. 11. 5′ amine and 5′ DBCO modified crRNAs showed levels of Cpf1 activity similar to that of unmodified crRNA during the in vitro cleavage assay. 5′ DNA modified crRNA showed reduced Cpf1 activity. Asterisk shows 5′ DNA modified crRNA band. Cleavage product has 350 bp size.

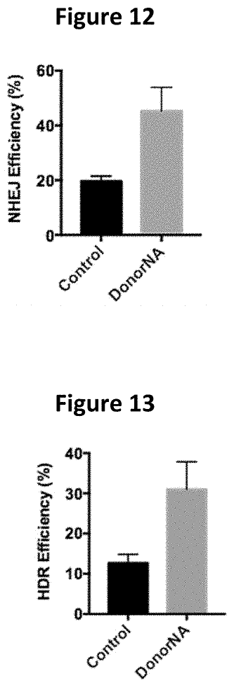

In another experiment, the 5′ end of crRNA was activated with thiopyridine to react with a thiol terminated donor DNA. A bridge DNA was used to facilitate the reaction. GFP-HEK cells were transfected with the crRNA-donor conjugate and Cpf1 protein using a cationic polymer encapsulation (pAsp(DET)). As a control, GFP-HEK cells were transfected in the same manner with crRNA, donor DNA, and Cpf1 without conjugation of the crRNA and donor DNA. NHEJ efficiency was determined based on GFP knock-out, and the results are shown in FIG. 12. HDR efficiency was determined based on a restriction enzyme digestion assay, as Donor DNA contained a Clal restriction enzyme site. The results are shown in FIG. 13.

The results demonstrate that the Cpf1-donor conjugate edited the target DNA with greater efficiency than the control.

### Example 7. Enzymatic Ligation of gRNA and Donor DNA

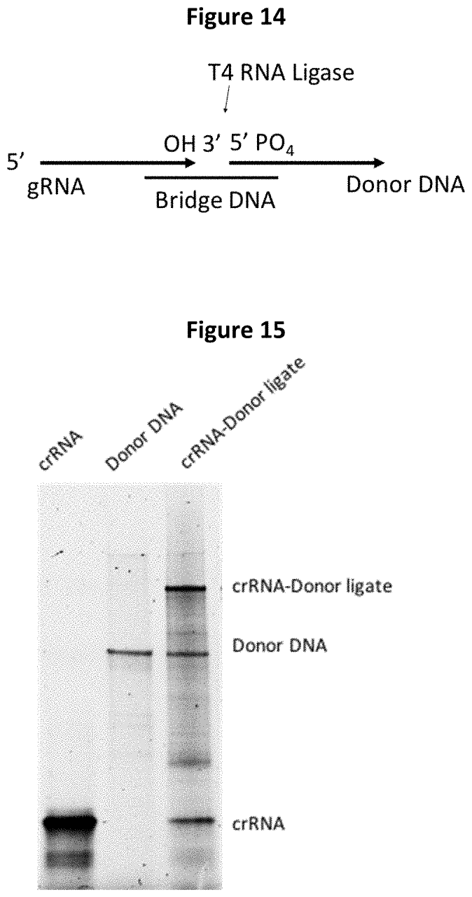

We ligated the 3′ end of crRNA and 5′ end of Donor DNA according to the scheme shown in FIG. 14. Using T4 RNA ligase-1, the crRNA and Donor DNA were successfully ligated using a bridge DNA. Bridge DNA hybridizes to both the 3′ end of crRNA and 5′ end of Donor DNA. One requirement for the reaction is an OH group on the 3′ end of the first nucleic acid and a phosphate group on the 5′ of the second nucleic acid. Ligation was confirmed by gel electrophoresis, as shown in FIG. 15. The crRNA-Donor DNA ligate band was gel extracted for purification.

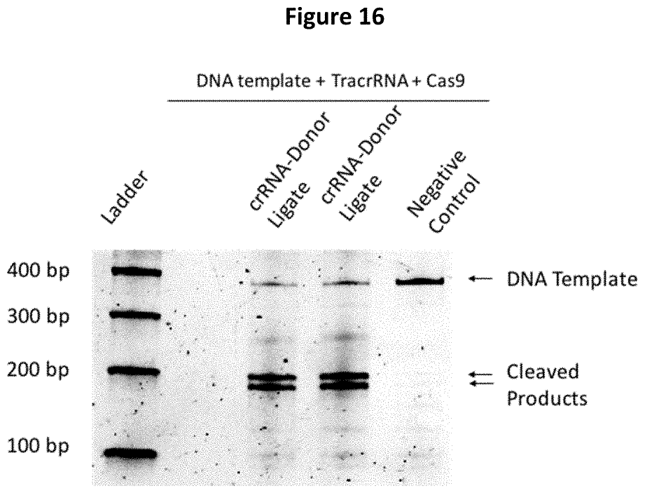

The enzymatically ligated crRNAs were complexed with Cas9 to test their cleavage activity with a model DNA template. 400 bp DNA template has a target sequence that is cleaved by crRNA/TracrRNA-Cas9. As a negative control, model DNA template without crRNA was used. Results were analyzed by gel electrophoresis, as presented in FIG. 16. The in vitro cleavage assay showed efficient cleavage of DNA template with the crRNA-Donor DNA ligates.

### Example 8. Rolling Circle Amplification of gRNA or Donor DNA

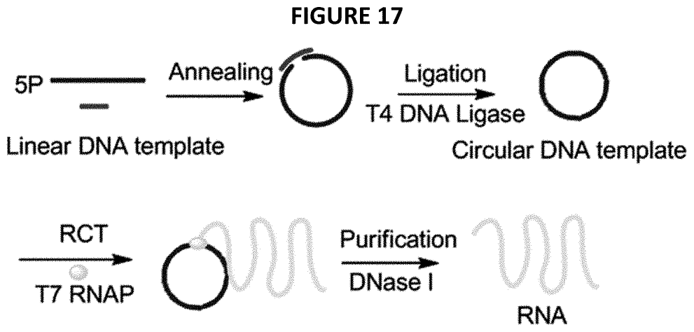

Currently used Cas9 gRNA is about 100 nt size. One interesting concept is delivering multiple RNPs at the same time. If we make long gRNA (IgRNA) with multiple repeats of gRNA and Cas9, it will be very efficient for delivery to cells and editing genes. The potential advantage of rolling circle amplified RNA (RC RNA) (FIG. 17) is that even delivering one RC RNA with high molecular weight can result in hundreds of desired gRNAs in cells after delivery. One RC RNA containing 100 gRNA repeats can potentially be cleaved into 100 single gRNAs in cells. It can be a very efficient way to deliver high concentration of gRNA into target cells. This same technique can be employed with Donor DNA as well. The idea is to have multiple repeats of donor DNA and increase the possibility of delivering a larger amount of donor DNA to a cell and have higher HDR.

Linear DNA template that contains a T7 promoter and a gRNA sequence targeting yellow fluorescent protein (YPF) with 5′ phosphate modification was purchased from IDT. T7 promoter DNA was hybridized to a linear DNA template by thermal denaturation and hybridization. T4 DNA ligase was incubated to make a circular DNA template. The template was incubated with exonuclease for 3 hr to remove linear DNA fragments. The circular DNA template was purified by ethanol precipitation, and the pure circular DNA template was incubated with T7 polymerase for 12 hr to synthesize the IgRNA by rolling circle amplification. RNA purification was conducted with Megaclear kit.

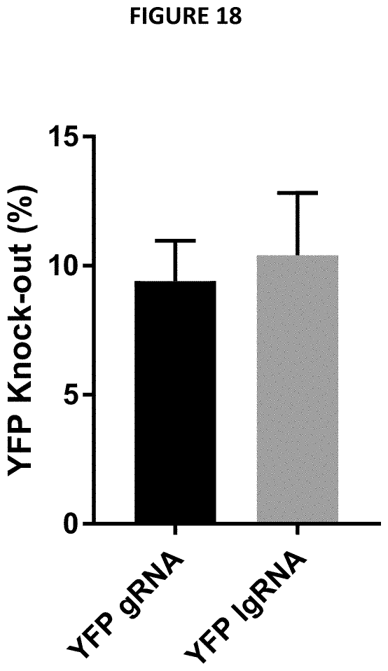

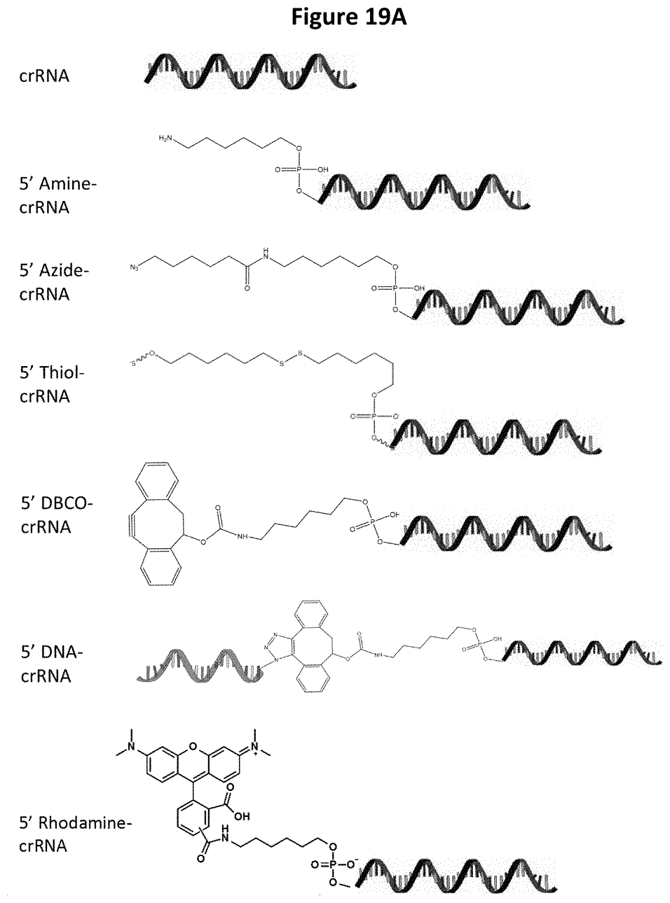

Nucleofection was conducted with YFP sgRNA (2 ug) or YFP IgRNA (2 ug) together with Cas9 protein (8 ug) into YFP expressing HEK293T cells. Flow cytometry was conducted 7 days after the nucleofection and FlowJo was used to quantify YFP knock-out percentage. The results are presented in FIG. 18, which shows YFP IgRNA worked as efficiently as regular YFP sgRNA when the same weight of each gRNA was delivered. Thus, IgRNA is functionally active in cells.

### Example 9. Conjugation of Single Guide RNA (sgRNA) and Donor DNA

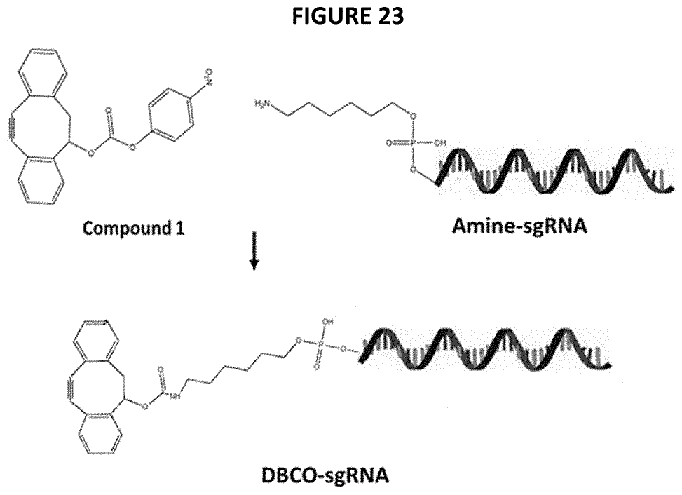

DBCO-modified sgRNA targeting the BFP gene was prepared as follows: 5′ Amine-sgRNA (100 μM) was suspended in a 100 μL of DMSO and mixed with a 100 fold molar excess of Compound 1 (10 mM). The reaction was incubated at room temperature for 16 hours and then purified with a desalting column (Micro Bio-Spin 30, Bio-rad). The concentration of the purified DBCO-sgRNA was measured with a Nanodrop. The reaction scheme is depicted in FIG. 23.

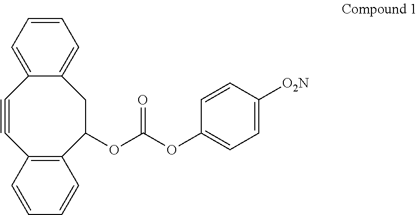 [SMILES: O=C(Oc1ccc([N+](=O)[O-])cc1)OC1Cc2ccccc2C#Cc2ccccc21]

The sgRNA was conjugated to donor DNA encoding GFP using copper-free click chemistry of azide and strained alkyne reaction. 5′ Azide-DNA Donor (15 μM) (which can be prepared using NHS-ester-amide) was mixed with 5′ DBCO-sgRNA (10 μM) in DI water (50 μL). The solution was incubated at room temperature overnight. The sample was analyzed via gel electrophoresis using a polyacrylamide gel (4-20% Mini-protean TGX Precast gel, Biorad). PAGE gel extraction was conducted to purify the sgRNA-Donor conjugate. The DNA-crRNA band was cut with a sharp knife and eluted using the crush and soak method in nuclease-free water for 16 hr, and isolated via ethanol precipitation. 200 ng of sgRNA, Donor DNA, and sgRNA-Donor DNA were analyzed via gel electrophoresis using a polyacrylamide gel to confirm the conjugation.

The purified sgRNA-Donor DNA conjugate was tested by nucleofection in BFP-HEK cells. Cells with no sgRNA were used as a control. The BFP-HEK cells were detached by 0.05% trypsin or gentle dissociation reagent, spun down at 600 g for 3 min, and washed with PBS. Nucleofection of the sgRNA/donor DNA conjugate was conducted using an Amaxa 96-well Shuttle system following the manufacturer's protocol, using 10 μL of Cas9 RNP. No sgRNA: Cas9—50 pmole, Donor DNA—60 pmole and sgRNA-Donor DNA: Cas9—50 pmole, sgRNA-Donor DNA conjugate—60 pmole. After the nucleofection, 500 μL of growth media was added and the cells were incubated at 37° C. in tissue culture plates. The cell culture media was changed 16 hours after the nucleofection, and the cells were incubated for 3 days. Then, fluorescence images were taken using a Zeiss inverted microscope and Zen 2015 software.

The results showed that, three days after the nucleofection, many cells expressed GFP and significant green fluorescence was observed, which indicates Cas9 cutting of the target BFP gene in the BFP-HEK cells and repair with donor DNA encoding GFP. The results demonstrate that sgRNA can be conjugated to Donor DNA while retaining gene editing activities.

### Example 10. Guide RNA Modification

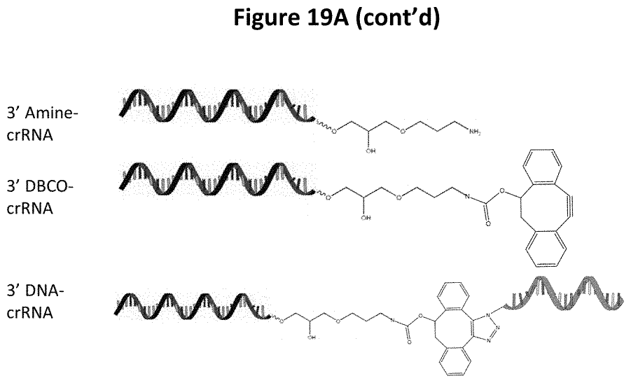

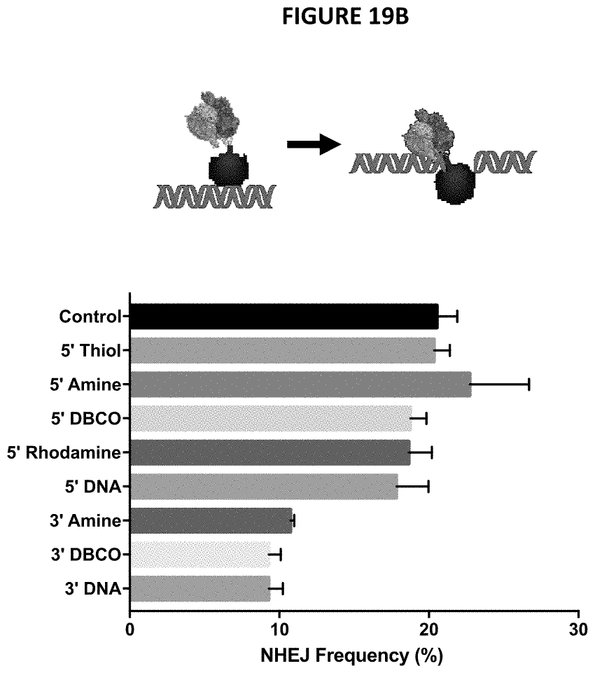

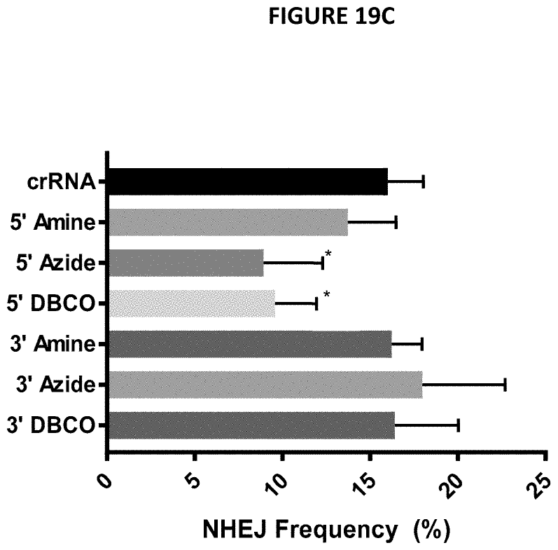

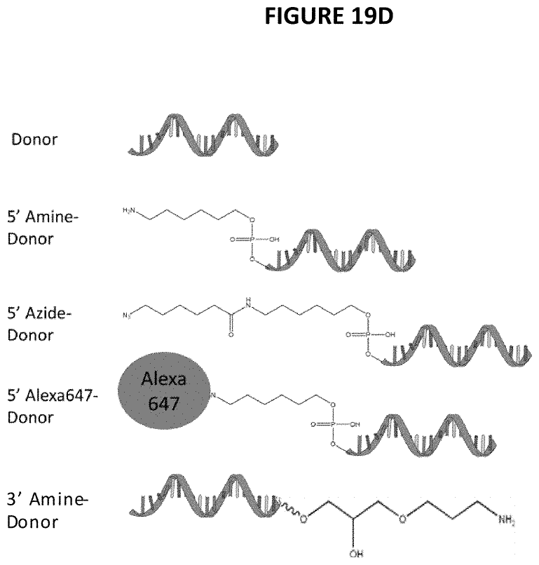

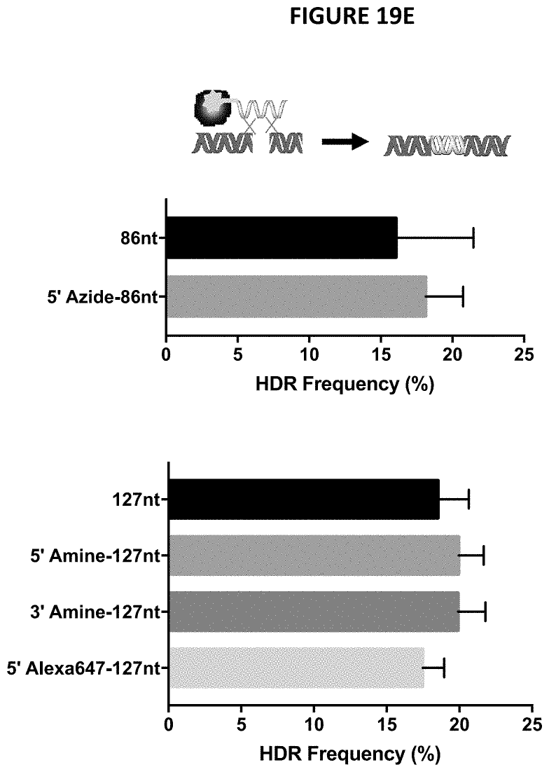

A library of 8 chemically modified CRISPR targeting RNAs (crRNAs) with modifications at the 5′ or 3′ end were created, and their ability to cleave DNA with Cas9 in cells expressing blue fluorescent protein (BFP) was analyzed. The chemical modifications were as shown in FIG. 19A. The library consisted of crRNAs targeting the BFP sequence, which had an amine, azide, fluorescent dye, strained alkyne, disulfide, or a short (127 nt) single stranded DNA at the 5′ or 3′ position. These modifications were chosen because of their importance in performing conjugation reactions and also because they represent a wide chemical space in terms of hydrophobic/hydrophilic balance and molecular dimensions.

The modified crRNAs were electroporated into cells along with tracrRNA and Cas9, which silences the BFP gene via an indel mutation. Thereafter, the percentage of BFP negative cells was determined via flow cytometry. The results presented in FIG. 19B show that the 5′ modified crRNAs had similar activity to unmodified crRNA, which is measured by non-homologous end joining (NHEJ) frequency in BFP-HEK and BFP-K562 cells. The crRNA with 3′ modifications had an approximately 50% reduction in NHEJ efficiency in cells, yet were still functional. Thus, the crRNA for Cas9 tolerates large modifications at its 5′ end very well, and is more sensitive to modifications on the 3′ end, yet still functional.

The tolerance of the Cpf1 guide RNA to chemical modifications also was investigated. Cpf1 is a recently discovered RNA-guided endonuclease of the class 2 CRISPR-Cas, and has the potential to be an alternative to Cas9 and edits sequences that do not have classical PAM sequences. Unlike Cas9, which requires both crRNA and tracrRNA, Cpf1 requires only crRNA, and this makes it an even more attractive target for chemical modifications.

BFP gene targeting crRNA along with Cpf1 was electroporated and the percentage of BFP negative cells was quantified with flow cytometry. The results presented in FIG. 19C demonstrate that the crRNA of AsCpf1 (from Acidaminococcus) tolerates chemical modifications at its 3′ end very well, and is more sensitive to 5′ end modifications. For example, BFP-HEK cells electroporated with 3′ amine-crRNA and Cpf1 had a similar NHEJ frequency as cells electroporated with Cpf1 and unmodified crRNA. BFP-HEK cells electroporated with crRNA with 5′ modifications still functional, but with reduced NHEJ frequency of 60-80% of NHEJ levels as cells treated with unmodified crRNA.

### Example 11. Donor DNA Modification

The tolerance of the donor DNA to chemical modifications was investigated. Donor DNA was modified at 5′ or 3′ termini with one of an azide, an amine, or Alexa 647 fluorescent dye. The results presented in FIG. 19D show the structures of the modifications.

For these experiments, a donor DNA encoding the GFP gene was used, and the modified donor DNA was electroporated into BFP-HEK cells along with Cas9 RNP targeting the BFP gene. Gene editing activity was assessed by GFP expression, which indicates HDR replacement of the BFP gene in the BFP-HEK cells with the GFP gene of the donor DNA.

The results presented in FIG. 19E show that BFP-HEK cells electroporated with the donor DNA modified at 3′ and 5′ ends were converted to GFP expressing cells via HDR. Thus, the donor DNA tolerates chemical modifications at both the 5′ and 3′ ends without loss of activity.

### Example 12. Enrichment Using Modified Donor DNA

The following example illustrates that labeled donor DNA can be used to provide a cell population enriched for those cells most likely to exhibit gene editing via HDR.

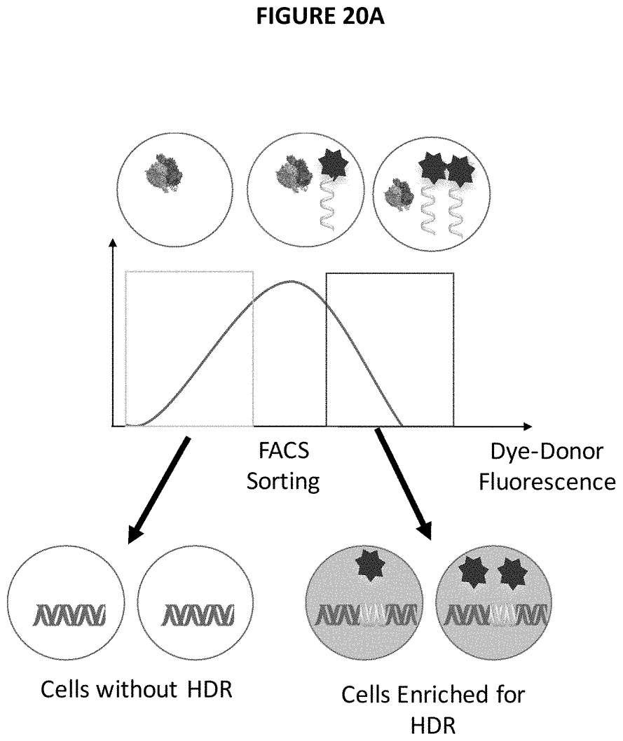

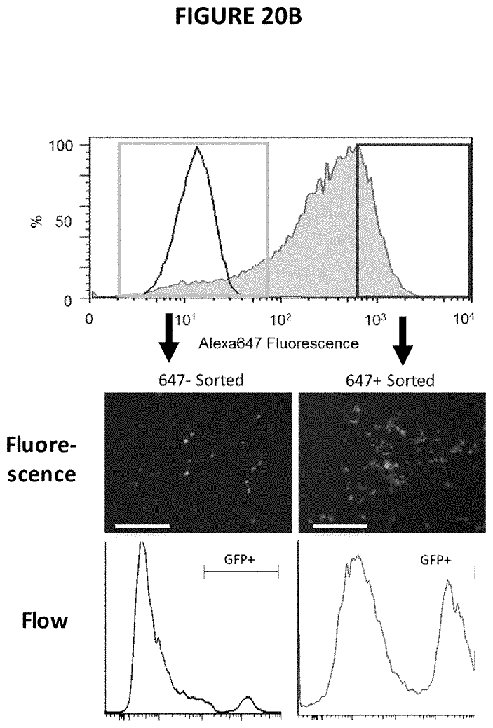

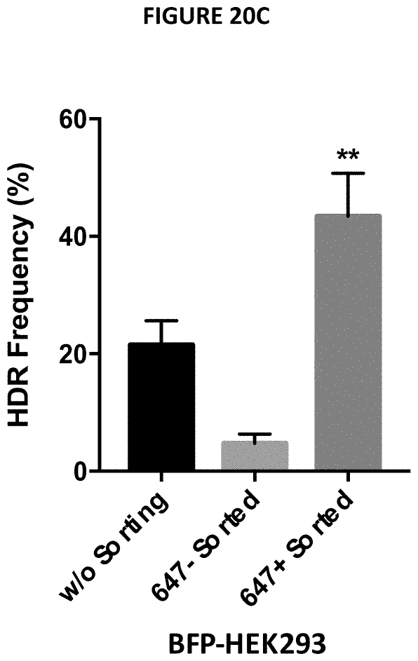

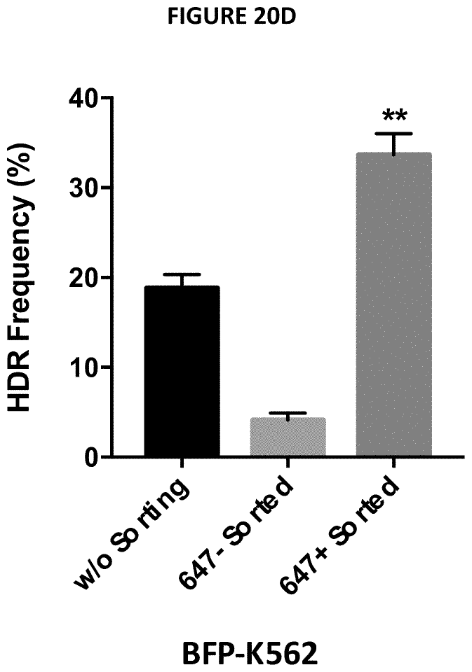

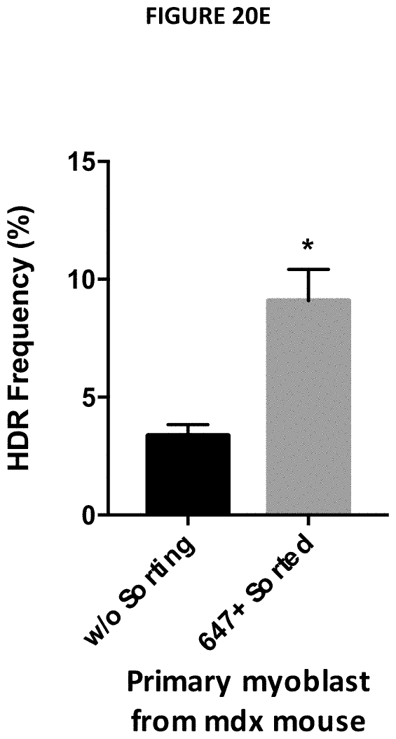

A Cas9 RNP that targets the BFP gene, and a donor DNA that converts the BFP gene to the GFP gene and was labeled with Alexa 647, termed trackable Donor (tDonor), were electroporated into BFP-HEK cells. 16 hours after the electroporation, cells that internalized high levels of the tDonor and low levels of the tDonor as indicated by Alexa 647 levels were sorted using fluorescence activated cell sorting (FACS), and cultured for 3 days. Flow cytometry was performed again on the cells, after three days of culturing, and the relative rates of HDR were determined and compared against bulk unsorted cells. FIG. 20A provides a general schematic of the method, and FIGS. 20B and 20C provide fluorescence data.

As illustrated in FIGS. 20B and 20C, BFP-HEK cells that had internalized high levels of the donor DNA also had a high rate of HDR. The HDR rate in these cells was enriched by a factor of 2, and reached close to 50%. The experiment was repeated using BFP-K562 cells with similar results (FIG. 20D).

Sorting cells based on the amount of donor DNA internalized also was able to identify primary cells that had been edited via HDR. Primary myoblasts from the Duchenne muscular dystrophy mouse model (mdx mice), which had a mutation in their dystrophin gene, were transfected with Cas9 RNP and a fluorescently labeled tDonor designed to correct the dystrophin mutation, using lipofectamine. The transfected cells were sorted via flow cytometry, using the fluorescence of the tDonor for gating, cultured, and analyzed for gene editing via restriction enzyme analysis. Results are provided in FIG. 20E, which demonstrate that the HDR rate in primary myoblasts with high levels of tDonor is two fold higher than unsorted cells. This shows that fluorescently labeled donor DNA represents an easy and fast method for enriching gene edited cells. The results show that labeled donor DNA provides an easy and fast method for enriching gene edited cells.

### Example 13. Guide-Donor Conjugate

A gRNA-donor DNA conjugate (gDonor) was synthesized by conjugating an azide terminated donor DNA with an alkyne modified crRNA, and hybridizing the resulting conjugate with tracrRNA. The gRNA was designed to cut the BFP gene and the donor DNA was designed to convert the BFP gene into the GFP gene.

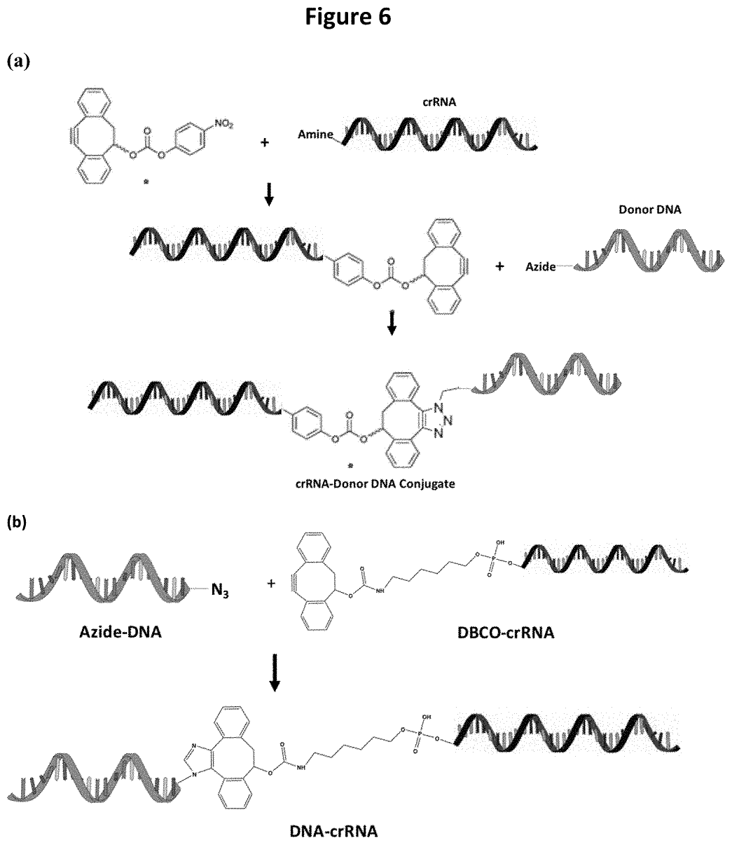

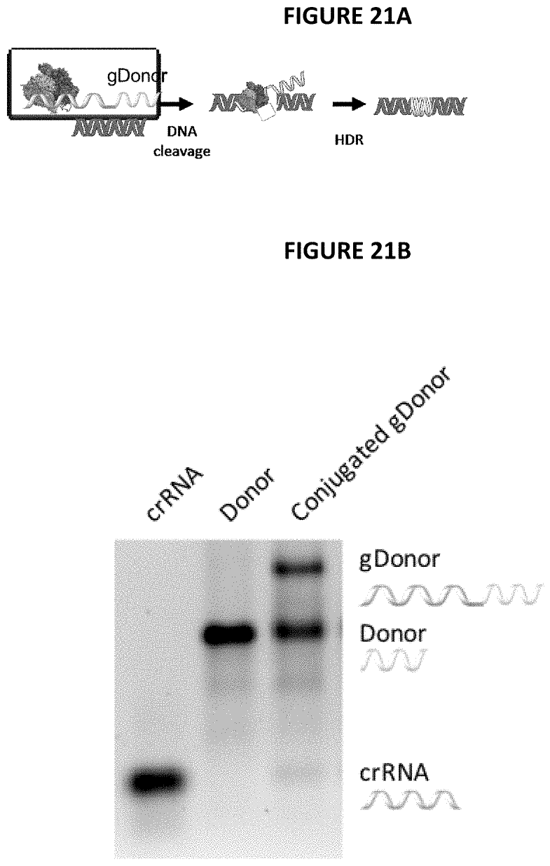

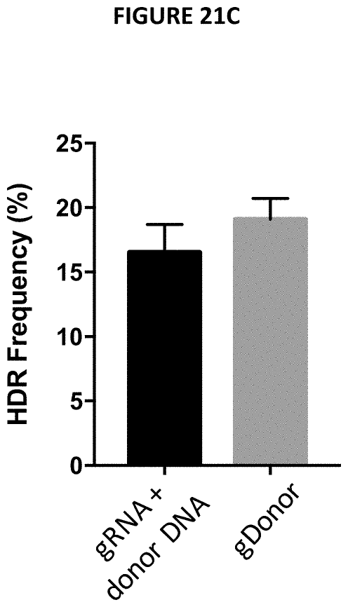

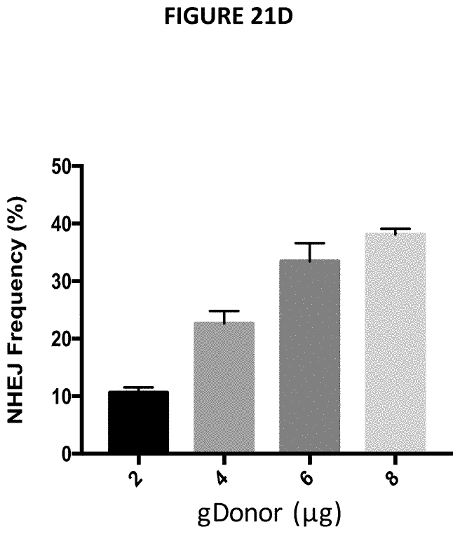

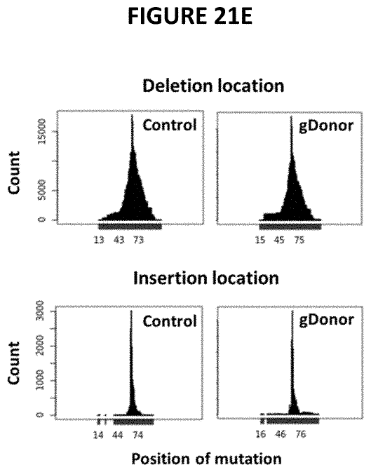

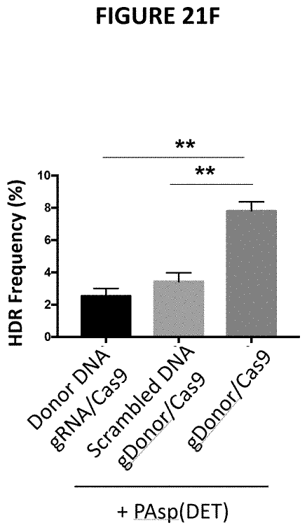

The conjugation step was based on copper-free click chemistry of azide and alkyne, as illustrated in FIG. 6. 5′ Azide-donor DNA (10 uM was mixed with 5′ DBCO-crRNA (10 uM) in DI water (50 uL). The solution was incubated at room temperature overnight. The gDonor was purified via gel extraction, and was synthesized with a 40% yield (FIG. 21B).

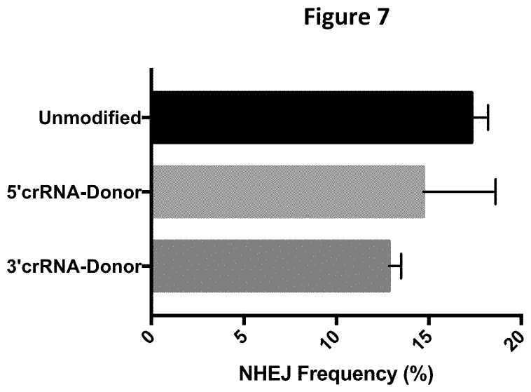

The activity of the gDonor was investigated by determining its ability to induce NHEJ or HDR in BFP-HEK cells, after electroporation with the Cas9 RNP. In addition, the DNA cleavage pattern of the gDonor in cells was also compared against cells treated with Cas9 RNP and donor DNA to determine whether conjugation to the donor DNA affected the function of the gRNA. Cells also were analyzed with flow cytometry 3 days after the transfection. FIG. 8 shows that 5′crRNA-Donor and 3′crRNA-Donor induces efficient HDR. FIG. 21C demonstrates that the gDonor was able to convert the BFP gene to the GFP gene via HDR with an efficiency similar to unmodified gRNA and Donor DNA (not conjugated), and thus both the gRNA and donor DNA of the gDonor are active. FIG. 7 shows that 5′ crRNA-Donor conjugate induces similar levels of NHEJ frequency compared to unmodified crRNA. FIG. 21D demonstrates that the NHEJ frequency induced by gDonor is dose dependent. In addition, deep sequencing analysis of the electroporated cells demonstrates that the gDonor cleaved its target sequence in cells with specificity and induced a similar pattern of indel mutations as unmodified gRNA control (FIG. 21E).

These results demonstrate that the gDonor can efficiently function as both a gRNA and a donor DNA.

### Example 14. Polymer Nanoparticle Delivery of Guide-Donor Conjugate

This example demonstrates that the gDonor could efficiently induce HDR in cells after delivery with cationic polymers.

The cationic polymer, pAsp(DET), was selected as the initial polymer to deliver the gDonor because of its well established ability to deliver siRNA into cells and in vivo. The gDonor was mixed with Cas9 and complexed with pAsp(DET), and generated nanoparticles 150 nm in diameter that contained the Cas9-gDonor complex.

In particular, gDonor (5 mg in 10 mL), and TracrRNA (2 mg in 10 mL) were mixed in 80 mL of Cas9 buffer (50 mM Hepes (pH 7.5), 300 mM NaCl, 10% (vol/vol) glycerol, and 100 mM TCEP), and hybridized by incubating at 60° C. for 5 min at RT for 10 min. Cas9 (8 mg in 10 mL) was added and incubated for 5 min at RT, and this solution was then added to the PAsp(DET) (10 mg in 20 mL) and incubated for 5 min at RT to generate polymer nanoparticles.

For characterization of the particles, the polymer nanoparticles were centrifuged at 17,000 g for 10 min, and the supernatant and pellet were collected. Each sample was mixed with a 100 mg of heparin for particle dissociation. The collected supernatant and pellets were run on a gel, and analyzed for the Cas9 and gDonor content in the polymer nanoparticles. Gel electrophoresis was performed using a 4-20% Mini-PROTEAN TGX Gel (Bio-rad) in Tris/SDS buffer, with a loading dye containing 5% beta-mercaptoethanol. PageBlue solution (Thermo Fisher) staining was conducted and imaged with ChemiDoc MP using ImageLab software (Bio-rad). For particle size measurements, a dynamic light scattering study was conducted using a Zetasizer Nano ZS instrument (Malvern Instruments Ltd., Worcestershire, UK) and a folded capillary cell (DTS 1060, Malvern Instruments). The reported particle size was measured 5 min after particle mixing.

The particles were added to BFP-HEK cells (105 cells) at a Cas9 concentration of 16 mg/mL in 500 mL volume of culture medium for 16 hr. crRNA-TracrRNA/Cas9+donor DNA were complexed with PAsp(DET) as a control and scrambled DNA-crRNA-TracrRNA/Cas9 and donor DNA were complexed with PAsp(DET) as a second control. Cell transfections with the two control nanoparticles were conducted following the same protocol used for transfecting cells with gDonor and TracRNA.

The HDR efficiency was determined by flow cytometry 3 days after the nanoparticle treatment. The results are presented in FIG. 31F, and demonstrate that gDonor significantly improves the ability of cationic polymers to simultaneously deliver Cas9, gRNA and donor DNA into cells. For example, the Cas9-gDonor complexed with pAsp(DET) induced an 8% HDR frequency in BFP-HEK cells, which was three times higher than that of the free gRNA and donor DNA complexed to pAsp(DET).

Additional control cell experiments were conducted with a scrambled DNA conjugated gRNA, which had the same charge density as the gDonor. Cells were treated with the scrambled DNA-crRNA/Cas9 complexed with pAsp(DET) and a separate complex of donor DNA/pAsp (DET), and the HDR efficiency was measured. FIG. 31F shows that the scrambled DNA-crRNA conjugate did not improve the transfection efficiency of pAsp(DET), suggesting that the gDonor's ability to enhance the efficacy of pAsp(DET) is not related to stronger complexation.

The gDonor, therefore, efficiently delivers both Cas9 RNP and donor DNA into cells.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A complex comprising a CRISPR system comprising an RNA-guided endonuclease, a guide RNA, and a donor polynucleotide, wherein the guide RNA and the donor polynucleotide are covalently or non-covalently linked.

2. The complex according to claim 1, wherein the CRISPR system is a Type II CRISPR system, and the RNA-guided endonuclease comprises a Cas9 polypeptide, or wherein the CRISPR system is a Type V CRISPR system, and the RNA-guided endonuclease comprises Cpf1 polypeptide.

3. (canceled)

4. The complex according to claim 1, wherein the guide RNA and the donor polynucleotide are covalently linked.

5. The complex according to claim 1, wherein the guide RNA and the donor polynucleotide are ligated.

6. The complex according to claim 5, wherein the guide RNA and the donor polynucleotide are hybridized to each other.

7. The complex according to claim 5, wherein the guide RNA and the donor polynucleotide are each hybridized to a bridge nucleic acid.

8. The complex according to claim 1, wherein the guide RNA is covalently linked to the donor polynucleotide, and the linkage comprises a triazole group or cyclic alkene group.

7. The complex according to claim 1, wherein the guide RNA is covalently linked to the donor polynucleotide, and the linkage comprises an amide bond or disulfide bond.

8. The complex of claim 1, wherein the donor polynucleotide is conjugated to the 3′ terminus of the guide RNA.

9. The complex of claim 1, wherein the donor polynucleotide is conjugated to the 5′ terminus of the guide RNA.

10. The complex according to claim 1, encapsulated in a cationic polymer or liposome.

11. The complex according to claim 10, wherein the cationic polymer is an endosomal disruptive polymer; optionally where the endosomal disruptive polymer is selected from the group consisting of polyethylene imine, poly(arginine), poly(lysine), poly(histidine), poly-[2-{(2-aminoethyl)amino}-ethyl-aspartamide] (pAsp(DET)), a block co-polymer of poly(ethylene glycol) (PEG) and poly(arginine), a block co-polymer of PEG and poly(lysine), and a block co-polymer of PEG and poly{N—[N-(2-aminoethyl)-2-aminoethyl]aspartamide} (PEG-pAsp(DET)).

12. The complex according to claim 11, wherein the endosomal disruptive polymer comprises poly{N—[N-(2-aminoethyl)-2-aminoethyl]aspartamide} (pAsp(DET).

13. The complex according to claim 12, wherein the complex further comprises a silicate.

14. The complex according to claim 2, wherein the Cas9 polypeptide comprises an amino acid sequence having at least 75% amino acid sequence identity to SEQ ID NO:1 or SEQ ID NO: 2.

15. (canceled)

16. The complex according to claim 1, wherein the guide RNA is a single-molecule guide RNA.

17. The complex according to claim 1, wherein the guide RNA is a dual-molecule guide RNA comprising a crRNA and tracrRNA, and the donor polynucleotide is linked to the crRNA.

18. A method of producing the complex of claim 1, the method comprising:
a) combining i) an RNA-guided endonuclease and ii) a guide RNA linked to a donor polynucleotide, to generate a complex; and
b) encapsulating the complex within one or more layers of an endosomal disruptive polymer.

19. A method of genetically modifying a eukaryotic target cell, comprising contacting the eukaryotic target cell with the complex according to claim 1, wherein the complex enters the cell, and wherein the guide RNA, donor polynucleotide and the RNA-guided endonuclease are released from the complex in an endosome in the cell.

20.-21. (canceled)

22. A complex comprising a CRISPR system comprising an RNA-guided endonuclease and a guide RNA, wherein the guide RNA is modified at the 3′ or 5′ terminus by amine, thiol, azide, tetrazine, alkyne, strained alkyne, strained alkene, detectable label or affinity tag, a peptide, or a nucleic acid;
or
a complex comprising a CRISPR system comprising an RNA-guided endonuclease, a guide RNA and a donor polynucleotide, wherein the guide RNA is modified at the 3′ or 5′ terminus by amine, thiol, azide, tetrazine, alkyne, strained alkyne, strained alkene, detectable label or affinity tag, a peptide, or a nucleic acid;
or
a guide RNA covalently linked to a donor nucleic acid, optionally via a linkage comprising a triazole or cyclic alkene group;
or
a guide RNA modified at the 3′ terminus or 5′ terminus with an amine, thiol, azide, tetrazine, alkyne, strained alkyne, or strained alkene group, a detectable label or affinity tag, a peptide, or a nucleic acid;
or
a guide RNA comprising a nucleotide extension sequence at the 3′ or 5′ end thereof, optionally wherein the nucleotide extension comprises about 10 nucleotides or more, and fewer than about 1000 nucleotides.

23.-26. (canceled)

27. The guide RNA of claim 26, wherein the guide RNA is a Cas9 single guide RNA, and the Cas9 single guide RNA is modified at the 5′ terminus with an amine, thiol, azide, tetrazine, alkyne, strained alkyne, or strained alkene group, a detectable label or affinity tag, a peptide, or a nucleic acid, or linked at the 5′ terminus to a DNA molecule.

28.-30. (canceled)

31. A method of screening for compounds that enhance the activity of an RNA-guided endonuclease, the method comprising:
(a) linking a test compound to the guide RNA of claim 22;
(b) combining (i) the guide RNA linked to the test compound; (ii) an RNA-guided endonuclease; (iii) a target DNA; and optionally (iv) a donor DNA; and
(c) selecting the test compound as enhancing the activity of the RNA-guided endonuclease if the guide RNA linked to the test compound produces enhanced gene editing of the target DNA as compared to the guide RNA without the test compound;
optionally wherein:
the guide RNA is modified with an azide or tetrazine group and the test compound comprises, or is modified to comprise, an alkyne, strained alkyne, or strained alkene group that reacts with the azide or tetrazine group to link the test compound to the guide RNA; or the guide RNA comprises an alkyne, strained alkyne, or strained alkene group and the test compound comprises, or is modified to comprise, an azide or tetrazine group that reacts with the strained alkyne group to link the test compound to the guide RNA.

32.-33. (canceled)

34. The method of claim 31, wherein the method further comprises
providing a library of test compounds each comprising an azide, tetrazine, alkyne, strained alkyne, or strained alkene functional group; and, for each test compound in the library:
(a) linking the test compound to the guide RNA;
(b) combining (i) the guide RNA linked to the test compound; (ii) an RNA guided endonuclease; (iii) a target DNA; and optionally (iv) a donor DNA; and
(c) selecting the test compound as enhancing the activity of the RNA-guided endonuclease if the guide RNA linked to the test compound produces enhanced gene editing of the target DNA as compared to the guide RNA without the test compound
or
providing a library of test compounds each linked to the guide RNA and, for each test compound in the library:
(a) combining (i) the guide RNA linked to the test compound; (ii) an RNA guided endonuclease; (iii) a target DNA; and optionally (iv) a donor DNA; and
(b) selecting the test compound as enhancing the activity of the RNA-guided endonuclease if the guide RNA linked to the test compound produces enhanced gene editing of the target DNA as compared to the guide RNA without the test compound.

35.-49. (canceled)

50. A composition comprising a guide RNA of claim 22 and a liposome, a polymer, or both, wherein the composition optionally further comprises an RNA guided endonuclease protein, or an mRNA encoding an RNA guided endonuclease protein, and/or a donor nucleic acid.

51.-53. (canceled)
